(19) United States
(12) Patent Application Publication
(10) Pub. No.: US 2022/0184020 A1
(43) Pub. Date: Jun. 16, 2022

(54) Polyphenols as Modulators of Platelet Function

(71) Applicants: Northeastern University, Boston, MA (US); The Brigham and Women's Hospital, Inc., Boston, MA (US)

(72) Inventors: Italo Faria do Valle, Boston, MA (US); Albert-László Barábasi, Brookline, MA (US); Joseph Loscalzo, Boston, MA (US); Harvey George Roweth, Boston, MA (US); Michael William Malloy, Boston, MA (US); Elisabeth M. Battinelli, Chestnut Hill, MA (US)

(21) Appl. No.: 17/558,402

(22) Filed: Dec. 21, 2021

Related U.S. Application Data
(63) Continuation of application No. 17/450,446, filed on Oct. 8, 2021.
Application No. 17/450,446 is a continuation-in-part of application No. 17/595,185, filed on Nov. 10, 2021.
(60) Provisional application No. 63/090,161, filed on Oct. 9, 2020.
(60) Provisional application No. 62/852,800, filed on May 24, 2019.

(51) Int. Cl. A61K 31/216; A61K 31/192; A61K 31/122; A61P 7/02

(52) U.S. Cl. CPC A61K 31/216; A61P 7/02; A61K 31/122; A61K 31/192

(57) ABSTRACT

Provided herein are methods of treating a vascular disease or condition in a subject in need thereof, comprising administering to the subject an effective amount of a vascular disease associated polyphenol (e.g., rosmarinic acid), or a pharmaceutically acceptable salt thereof. Also provided herein are methods of promoting or supporting vascular health in a subject, and methods of inhibiting platelet function (e.g., platelet aggregation) in a subject.

### RELATED APPLICATION

This application is a continuation of U.S. patent application Ser. No. 17/450,446, filed on Oct. 8, 2021, which claims the benefit of U.S. Provisional Application No. 63/090,161, filed on Oct. 9, 2020, and a continuation-in-part of U.S. patent application Ser. No. 17/595,185, filed on Nov. 10, 2021, which is the U.S. National Stage of International Application No. PCT/US2020/034299, filed on May 22, 2020, which designates the U.S., published in English, and claims the benefit of U.S. Provisional Application No. 62/852,800, filed on May 24, 2019. The entire teachings of these applications are incorporated herein by reference.

### GOVERNMENT SUPPORT

This invention was made with government support under 1P01HL132825, awarded by National Institutes of Health. The government has certain rights in the invention.

### BACKGROUND

Diet can be a key environmental factor that affects human health—while poor diet can significantly increase the risk for coronary heart disease (CHD) and diabetes, a healthy diet can play a protective role, even mitigating genetic risk of CHD. Polyphenols are a class of compounds that can play a protective role for a wide range of diseases, from cancer to diabetes mellitus, as well as for cardiovascular and neurodegenerative diseases. Polyphenols can act as antioxidants and are present in plant-based foods, such as fruits, vegetables, herbs, spices, teas, and wine. Polyphenols are characterized by multiples of phenolic or hydroxy-phenolic structural features, and most contain repeating phenolic moieties of resorcinol, pyrocatechol, pyrogallol, and phloroglucinol linked by ester or carbon-carbon bonds. Recent efforts profiling over 500 polyphenols in more than 400 foods have documented the high diversity of polyphenols humans are exposed to through their diet, ranging from flavonoids to phenolic acids, lignans, and stilbenes.

While polyphenols, as one example of a class of chemical compounds that can affect human health, are generally known to provide for healthful effects, underlying molecular mechanisms through which specific polyphenols exert their function, as well as associations with particular diseases, remain largely unexplored.

Accordingly, there is a need for identifying networks of polyphenols having an association to a particular disease or condition, such as a vascular disease or condition, and using the identified networks to treat the disease or condition.

### SUMMARY

Methods for treating a vascular disease or condition in a subject in need thereof are provided. Also provided are methods for promoting or supporting vascular health in a subject (e.g., a subject in need thereof), and methods for modulating (e.g., inhibiting) platelet function in a subject (e.g., a subject in need thereof). The methods comprise administering to the subject an effective amount of a vascular disease associated polyphenol, or a pharmaceutically acceptable salt thereof.

A method of inhibiting platelet function is also described. The method comprises contacting platelets with a vascular disease associated polyphenol, or a pharmaceutically acceptable salt thereof.

Also described are systems and methods that can be used as tools in providing for the identification of diseases affected by a given chemical or class of chemicals, such as polyphenols. The systems and methods described can provide for mechanistic insight as to the molecular pathways responsible for the health implications of a chemical.

A method of identifying a disease associated with a therapeutic chemical includes generating a candidate disease list based on proximities of proteins associated with a plurality of diseases and proteins associated with a therapeutic chemical in a protein-protein interaction network. The method further includes applying gene expression information associated with the therapeutic chemical to generate enrichment scores for diseases of the candidate disease list and identifying at least one disease associated with the therapeutic chemical based on the determined enrichment scores.

A method of filtering data in a protein-protein interaction network includes mapping proteins associated with a plurality of diseases and proteins associated with a therapeutic chemical. The method further includes determining proximities of proteins associated with the plurality of diseases and proteins associated with the therapeutic chemical. An enrichment score is generated for each of the plurality of diseases based on gene expression information associated with the therapeutic chemical. A reduced dataset of proteins within the protein-protein interaction network is generated, the reduced dataset of proteins being proteins associated with a subset of the plurality of diseases based on the determined proximities and the determined enrichment scores. The subset of diseases can be a candidate disease list.

Generating a candidate disease list can include generating a proximity value for a disease and the therapeutic chemical. Determining proximities, or determining a proximity value, can be based on shortest path lengths between nodes representing proteins associated with the disease and nodes representing proteins associated with the therapeutic chemical in the protein-protein interaction network. The proximity value can be a distance metric, such as d_{c}(S,T) as given by the following:

$${d}_{c} ( S , T ) = \frac{1}{‖ T ‖} {Σ}_{t ∈ T} {min}_{s ∈ S} d ( s , t ) \qquad [ 1 ]$$

where S is a set of proteins associated with the disease, T is a set of proteins associated with the therapeutic chemical, s is a node representing a protein in set S, t is a node representing a protein in set T, and d(s,t) is a shortest path length between nodes s and t in the protein network.

Generating an enrichment score can include measuring an extent of gene expression perturbation by the therapeutic chemical for a disease, such as, for example, by performing a Gene Set Enrichment Analysis.

The methods can further include ranking the diseases of the candidate disease list based on the determined proximity and the determined enrichment scores. The protein-protein interaction network can be a human interactome. The proteins associated with a therapeutic chemical can be proteins to which the therapeutic chemical binds. For example, the therapeutic chemical can be a polyphenol and the proteins associated with the therapeutic chemical can be binding targets of the polyphenol.

A method of treating a subject having a disease includes administering a therapeutic chemical, wherein the disease is a disease identified by any of the method described above as being associated with the therapeutic chemical.

A system for identifying a disease associated with a therapeutic chemical includes a processor configured to generate a candidate disease list based on proximities of proteins associated with a plurality of diseases and proteins associated with a therapeutic chemical in a protein-protein interaction network. The processor is further configured to apply gene expression information associated with the therapeutic chemical to generate enrichment scores for diseases of the candidate disease list and to identify at least one disease associated with the therapeutic chemical based on the determined enrichment scores.

A system for filtering data in a protein-protein interaction network includes a processor configured to map proteins associated with a plurality of diseases and proteins associated with a therapeutic chemical and determine proximities of proteins associated with the plurality of diseases and proteins associated with the therapeutic chemical. The processor is further configured to generate an enrichment score for each of the plurality of diseases based on gene expression information associated with the therapeutic chemical and to generate a reduced dataset of proteins within the protein-protein interaction network, the reduced dataset of proteins being proteins associated with a subset of the plurality of diseases based on the determined proximities and the determined enrichment scores.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is diagram of a filter for reducing proteins of a protein-protein interaction network for a therapeutic chemical.

FIG. 2 is a diagram of a computer processor operation 100 for identifying a disease associated with a therapeutic chemical.

FIG. 3 is a schematic view of a computer network environment in which embodiments of the present invention may be deployed.

FIG. 4 is a block diagram of computer nodes or devices in the computer network of FIG. 3.

FIG. 5A is a schematic representation of an interactome, with highlighted regions where polyphenol targets and disease proteins are localized.

FIG. 5B is a diagram showing the selection criteria of the polyphenols evaluated in a study.

FIG. 5C is a distribution of the number of polyphenol protein targets mapped to the human interactome.

FIG. 5D is a graph of the top (n=15) enriched Gene Ontology (GO) pathways (Biological Process) among all polyphenol protein targets. The X axis shows the proportion of targets mapped to each pathway.

FIG. 5E is a plot of the size of the Largest Connected Component (LCC) formed by the targets of each polyphenol in the interactome and the corresponding significance (z-score).

FIGS. 6A-6C illustrate protein subgraphs of the targets of twenty-three polyphenols. The targets of the twenty-three polyphenols form connected components in the interactome. For example, piceatannol targets form a unique connected component of 23 proteins, while quercetin targets form multiple connected components, the biggest with 140 proteins. Polyphenol targets that are not connected to any other target are not shown in the figure.

FIG. 7A illustrates an interactome neighborhood showing EGCG protein targets and their interactions with type 2 diabetes (T2D)-associated proteins.

FIG. 7B is a distribution of AUC values considering the predictions of therapeutic effects for 65 polyphenols.

FIG. 7C is illustrates a comparison of the ECGC-disease associations considering the CTD database and the in-house database derived from the manual curation of the literature.

FIG. 7D is a graph of a comparison of the prediction performance when considering known EGCG-disease associations from the CTD, in-house manually curated database, or combined datasets.

FIG. 8A is a schematic representation of the relationship between the extent to which a polyphenol perturbs disease genes expression, its proximity to the disease genes, and its therapeutic effects.

FIG. 8B illustrates an interactome neighborhood showing the modules of Skin Diseases (SK), Genistein, and Cerebrovascular Disorders (CD). The SK module has 10 proteins with high perturbation scores (>2) in the treatment of the MCF7 cell line with 1 μM of genistein. Genes associated to SK are significantly enriched among the most differentially expressed genes, and the maximum perturbation score among disease genes is higher in SK than CD.

FIGS. 8C-1-8C-4 illustrate therapeutic associations for four polyphenols. Among the diseases in which genes are enriched with highly perturbed genes, those with therapeutic associations show smaller network distances to the polyphenol targets than those without. The same trend is observed in treatments of the polyphenols genistein (FIG. 8C-1), quercetin (FIG. 8C-2), resveratrol (FIG. 8C-3), and myricetin (FIG. 8C-4).

FIG. 9A is a schematic representation of proximal and distal diseases in relation to genistein targets. Each node represents a disease and the node size proportional to the perturbation score after treatment with genistein (1 μM, 6 hours). Distance from the origin represents the network proximity (dc) to genistein targets. Purple nodes represent diseases in which the therapeutic association was previously known.

FIGS. 9B-1-9B-4 illustrate cumulative distributions of the maximum perturbation scores of genes from diseases that are distal or proximal to polyphenol targets considering different polyphenols (1 μM, 6 hours): genistein (FIG. 9B-1), quercetin (FIG. 9B-3), resveratrol (FIG. 9B-2), and myricetin (FIG. 9B-4). Statistical significance was evaluated with the Kolmogorov Smirnov test.

FIG. 10 illustrates an interactome neighborhood containing the interactions between proteins associated with Vascular Diseases and the targets of 1,4-naphthoquinone, gallic acid, and rosmarinic acid.

FIG. 11A illustrates an interactome neighborhood showing Rosmarinic acid (RA) targets and the RA-VD-platelet module—the connected component formed by the RA target FYN and the VD proteins associated to platelet function PDE4D, CD36, and APP—and the receptor of platelet stimulants used in experiments (Collagen/CRPXL, TRAP6, U46619, and ADP).

FIG. 11B is a graph of average shortest path length from each platelet stimulant receptor and the RA-VD-platelet module formed by the proteins FYN, PDE4D, CD36, APP.

FIGS. 11C-1-11C-4 are graphs of assessed aggregation of platelets. Platelet-rich plasma (PRP) or washed platelets were pre-treated with RA for 1 hour before stimulation with either collagen (1 μg/mL, FIG. 11C-1), collagen-related peptide (CRP-XL, 1 μg/mL), thrombin receptor activator peptide-6 (TRAP-6, 20 μM, FIG. 11C-2), U46619 (1 μM, FIG. 11C-3), or ADP (10 μM, FIG. 11C-4).

FIGS. 11D-1-11D-4 are graphs of assessed alpha granule secretion of the platelets of FIG. 11C.

FIG. 11E illustrates results of protein tyrosine phosphorylation (P-Tyr) assessment of the platelets of FIG. 11C. Numbers on the right indicate protein molecular weight. N=3-6 separate blood donations, mean+/−SEM.

FIG. 11F illustrates results of protein tyrosine phosphorylation (P-Tyr) assessment of the platelets of FIG. 11C.

FIG. 12 shows number of disease associations and reference papers for the polyphenols evaluated. Comparison of the distribution of disease associations and reference papers between polyphenols not included (0) and included (1) in this study. P-values were obtained with the Mann-Whitney test.

FIG. 13 is a comparison of protein targets among polyphenol pairs measured by the Jaccard Index. The clustering was performed using the complete linkage method and the Euclidean distance metric.

FIGS. 14A and 14B illustrate target similarity among polyphenols. FIG. 14A shows the distribution of the similarity (Jaccard Index) of the protein targets among polyphenol pairs. FIG. 14B shows expected values of Jaccard Index (JI) average values if the targets of each polyphenol were randomly assigned from the pool of all network proteins with degrees matching the original set.

FIG. 15 is a comparison of enriched gene ontology pathways among polyphenol pairs measured by the Jaccard Index. The clustering was performed using the complete linkage method and the Euclidean distance metric.

FIG. 16 illustrates network proximity among polyphenol targets. It was asked whether the polyphenol targets spread through different regions of the interactome or are confined to specific network neighborhoods. The figure shows the distribution of the network proximity significance among targets of each polyphenol considering the average shortest path among all targets (SP) and the average shortest path to the nearest target (SPclosest), showing that the targets tend to be proximal to each other compared with random expectation, and that this proximity is even greater when considering the average of distances to the nearest protein.

FIGS. 17A-17D are a comparison of predictive performance considering the literature-derived interactome assembled in this study and an interactome derived from an unbiased high-throughput Screening. The largest connected component of the high-throughput derived interactome was considered, which consisted of 8,955 proteins and 63,619 protein-protein interactions. 49/65 polyphenols could be mapped in both interactomes, while 16/49 could be mapped only in the literature-derived interactome. FIG. 17A shows (−)-epicatechin, (−)-epicatechin 3-o-gallate, (−)-epigallocatechin 3-o-gallate, 1,4-naphthquinone, 2,3-dihydroxybenzoic acid, 2-hydroxybenzoic acid, 3-phenylpropionic acid, 4-methylcatechol, apigenin, baicalein, butein, caffeic acid. FIG. 17B shows chrysin, cinnamic acid, coumarin, coumestrol, daidzein, ellagic acid, esculetin, ferulic acid, galangin, gallic acid, galloyl glucose, genistein, guaiacol. FIG. 17C shows isoliquiritigenin, isorhamnetin, juglone, kaempferol, kaempferol 3-o-glucoside, luteolin, luteolin 6-o-glucoside, myricetin, p-coumaric acid, phenol, phenylacetic acid, phloridzin. FIG. 17D shows piceatannol, pterostilbene, quercetin, quercetin 3-o-glucoside, quercetin 3-o-glucuronide, quercetin 3-o-rutinoside, resveratrol, rosmarinic acid, scutellarein, theaflavin, umbelliferone, xanthotoxin.

FIG. 18 is a comparison of predictive performance considering the source of polyphenol protein interactions data. PDB provides binding evidence at the 3D resolution level. Proteins for 7 polyphenols were retrieved in PBD.

FIGS. 19A-19R illustrate enrichment of perturbated genes in expression profiles versus network proximity. Among diseases whose genes are enriched with highly perturbed genes, those with therapeutic associations show smaller network distances to the polyphenol targets than those without. Comparison of polyphenols genistein 500 μm (FIG. 19A), genistein 100 μm (FIG. 19B), genistein 10 μm (FIG. 19C), genistein 1 μm (FIG. 19D); quercetin 500 μm (FIG. 19E), quercetin 1 μm (FIG. 19F), quercetin 3 μm (FIG. 19G), quercetin 10 μm (FIG. 19H), myricetin 100 μm (FIG. 19I), myricetin 1 μm (FIG. 19J), myricetin 5 μm (FIG. 19K), myricetin 10 μm (FIG. 19L), myricetin 20 μm (FIG. 19M), resveratrol 100 μm (FIG. 19N), resveratrol 1 μm (FIG. 19O), resveratrol 5 μm (FIG. 19P), resveratrol 10 μm (FIG. 19Q), and resveratrol 20 μm (FIG. 19R).

FIGS. 20A-20I illustrate enrichment of perturbated genes in expression profiles versus network proximity. Among diseases whose genes are enriched with highly perturbed genes, those with therapeutic associations show smaller network distances to the polyphenol targets than those without. Comparison of polyphenols: (−)-epicatechin (FIG. 20A), (−)-epicatechin 3-O-gallate (FIG. 20B), apigenin (FIG. 20C), caffeic acid (FIG. 20D), coumarin (FIG. 20E), coumestrol (FIG. 20F), daidzein (FIG. 20G), isoliquiritigenin (FIG. 20H), and umbelliferone (FIG. 20I) at 10 μM.

FIGS. 21A-21P illustrate diseases proximal to the polyphenol have higher perturbation in expression profiles of the cell line MCF7 treated with the respective polyphenol. Each disease is represented by the perturbation score of its most perturbed gene in the expression profiles. The comparison of the distribution of proximal and distant diseases was evaluated using the Kolmogorov Smirnov test. Comparisons of polyphenols genistein 0.10 μM (FIG. 21A), genistein 0.50 μM (FIG. 21B), genistein 1.00 μM (FIG. 21C), genistein 10.00 μM (FIG. 21D), myricetin 0.10 μM (FIG. 21E), myricetin 5.00 μM (FIG. 21F), myricetin 1.00 μM (FIG. 21G), myricetin 10.00 μM (FIG. 21H), quercetin 0.50 μM (FIG. 21I), quercetin 1.00 μM (FIG. 21J), quercetin 3.00 μM (FIG. 21K), quercetin 10.00 μM (FIG. 21L), resveratrol 0.10 μM (FIG. 21M), resveratrol 1.00 μM (FIG. 21N), resveratrol 5.00 μM (FIG. 21O), and resveratrol 10.00 μM (FIG. 21P).

FIGS. 22A-22N illustrate diseases proximal to the polyphenol have higher perturbation in expression profiles of the cell line MCF7 treated with the respective polyphenol. Each disease is represented by the perturbation score of its most perturbed gene in the expression profiles. The comparison of the distribution of proximal and distant diseases was evaluated using the Kolmogorov Smirnov test. Comparisons of polyphenols narigenin 10.00 μM (FIG. 22A); caffeic acid 10.00 μM (FIG. 22B); (−)-epicatechin 3-O-gallate 10.00 μM (FIG. 22C); piceatannol 10.00 μM (FIG. 22D); isoliquiritigenin 10.00 μM (FIG. 22E); coumarin 10.00 μM (FIG. 22F); (−)-epicatechin 10.00 μM (FIG. 22G); pterostilbene 10.00 μM (FIG. 22H); umbelliferone 10.00 μM (FIG. 22I); coumestrol 10.00 μM (FIG. 22J); (−)-epigallocatechin 3-O-gallate 10.00 μM (FIG. 22K); apigenin 10.00 μM (FIG. 22L); daidzein 10.00 μM (FIG. 22M); and rosmarinic acid 10.00 μM (FIG. 22N).

FIGS. 23A-23I illustrate rosmarinic acid modulates NRF2 pathways. Rosmarinic acid induces higher perturbation of NRF2 targets in comparison to all other genes in the cell lines MCF7 and A549. FIG. 23A: PC3_10 uM_6 h; FIG. 23B: HCC515_10 uM_6 h; FIG. 23C: VCAP_10 uM_6 h; FIG. 23D: A375_10 uM_6 h; FIG. 23E: HEPG2_10 uM_6 h; FIG. 23F: A549_10 uM_6 h; FIG. 23G: HA1E_10 uM_6 h; FIG. 23H: MCF7_10 uM_6 h; and FIG. 23I: HT29_10 uM_6 h.

FIGS. 24A-24D illustrate rosmarinic acid modulates platelet dense granule release, integrin activation and tyrosine phosphorylation. Platelet-rich plasma (PRP) was pre-treated with RA for 1 hour before stimulation with either collagen (1 μg/mL), collagen-related peptide (CRP-XL, 1 μg/mL), thrombin receptor activator peptide-6 (TRAP-6, 10,20 μM), or U46619 (1 μM). Platelets were assessed for either dense granule secretion (FIG. 24A) or integrin αIIbβ3 activation (FIG. 24B). Arrows indicate the time of agonist addition. Grey histograms represent unstimulated samples, lightly shaded histograms represent samples with no RA pretreatment and filled histograms represent stimulation with prior RA treatment (100 μM). FIG. 24C shows washed platelets were pre-treated with rosmarinic acid (RA) for 1 hour and supernatants tested for lactate dehydrogenase (LDH). Boxed points indicate platelets lysed with Triton X-100, dashed line indicates basal LDH release from untreated platelets. FIG. 24D shows platelet lysates were probed with the antibody 4G10 to measure total tyrosine phosphorylation. N=1-6 separate blood donations, mean+/−SEM.

### DETAILED DESCRIPTION

A description of example embodiments follows.

Systems and methods are presented for identifying diseases whose proteins are candidates to show gene expression perturbation under a treatment with a given chemical compound. The systems and methods presented herein can function as a filter in a protein-protein interaction network, such as the human interactome, to reduce proteins present in the network to a subset of proteins associated with a chemical compound and a disease.

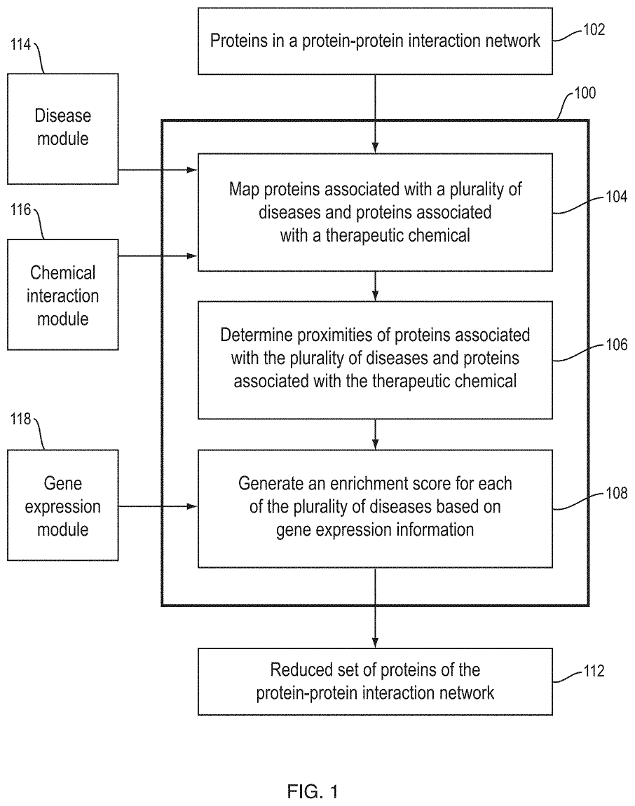

An example of a filter 100 that can be applied to a protein-protein interaction network 102 is shown in FIG. 1. From the proteins present in a protein-protein interaction network 102, the filter 100 functions to reduce the proteins present in the network to a subset of proteins that are associated with a chemical-disease relationship. Systems and methods including filter 100 operate by mapping proteins associated with a plurality of diseases and proteins associated with a therapeutic chemical (step 104). Information regarding proteins associated with one or more diseases can be provided from a disease module 114 to identify disease clusters within the protein-protein interaction network. Information regarding proteins associated with one or more chemicals can be provided by a chemical interaction module 116 to identify chemical target locations within the network. After mapping, the filter 100 determines proximities, within the network, of proteins associated with the plurality of diseases and proteins associated with the therapeutic chemical (step 106). Gene expression information is applied to generate an enrichment score for each of the one or more diseases under consideration (step 108). The gene expression information can be provided by a gene expression module 118 that includes perturbation signatures for cell lines treated with the one or more chemicals. Based on the determined proximities and enrichment scores, the proteins within the network are reduced to one or more sets 112 associated with a particular chemical-disease relationship.

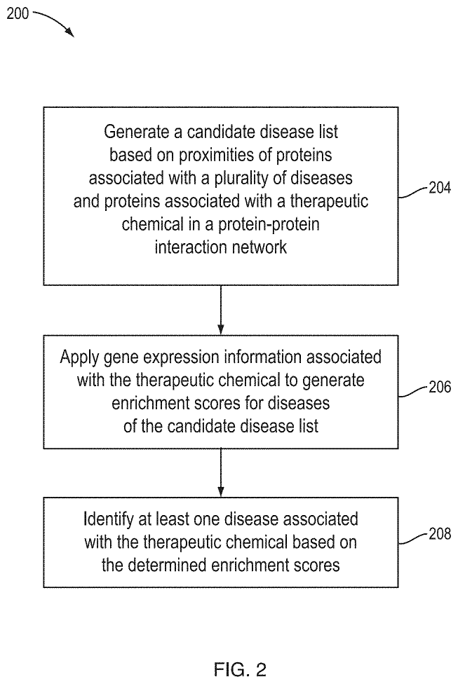

An example of a method 200 for identifying a disease associated with a therapeutic chemical is shown in FIG. 2. The method includes generating a candidate disease list based on proximities of proteins associated with a plurality of diseases and proteins associated with a therapeutic chemical in a protein-protein interaction network (step 204). Gene expression information can be applied to generate an enrichment score for diseases of the candidate disease list (step 206). From the determined enrichment scores of diseases in the candidate disease list, at least one diseases associated with the therapeutic chemical can be identified (step 208).

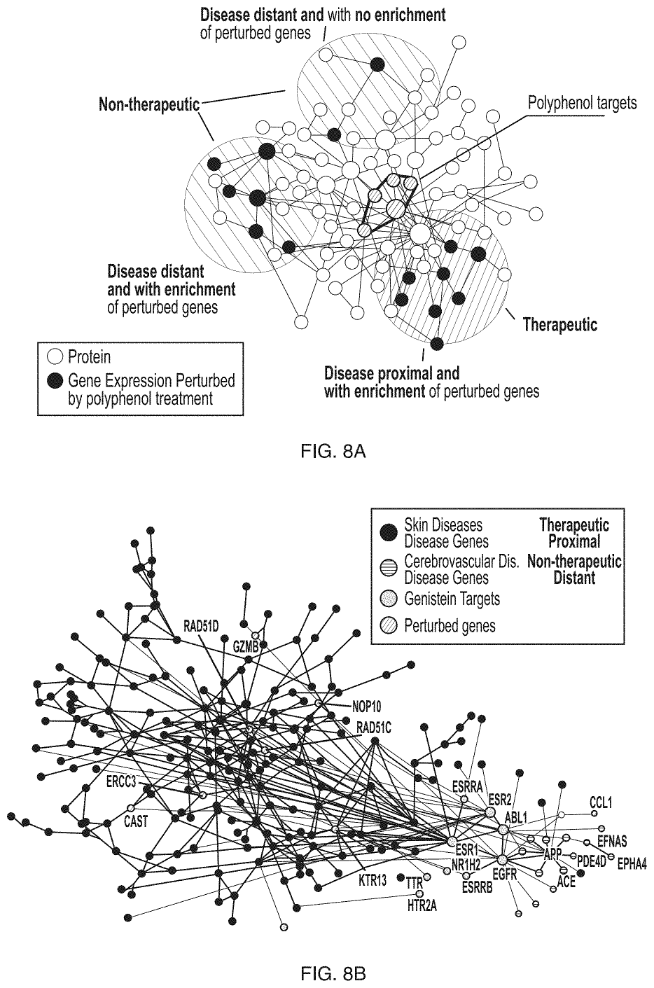

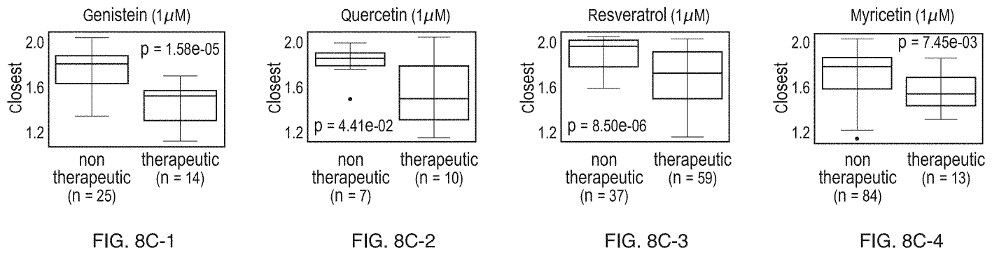

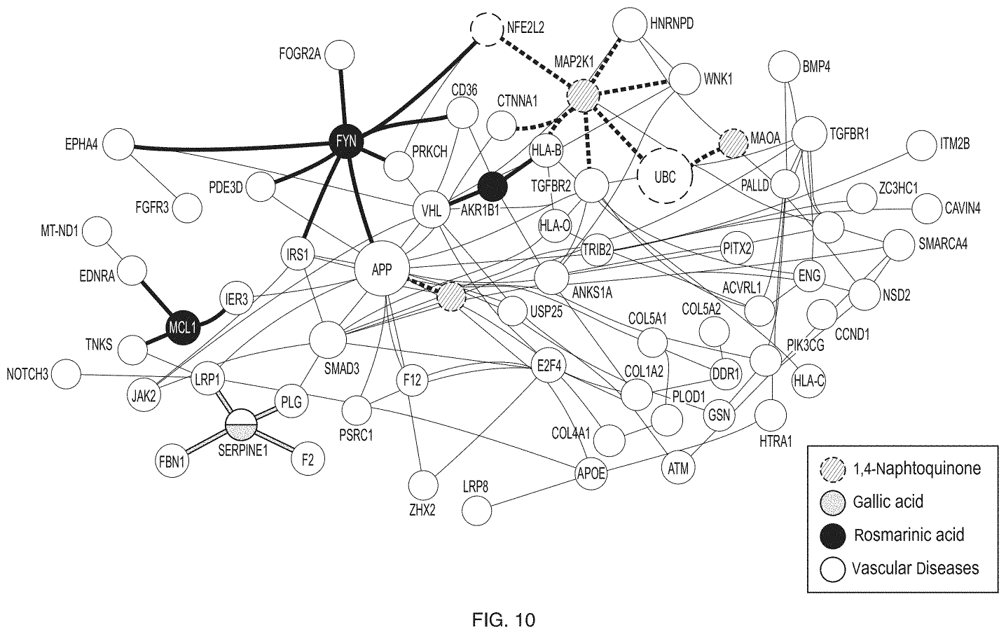

Example methods and systems for identifying a disease cluster within a protein network are described in WO2015/084461, the entire contents of which are incorporated herein by reference. Disease clusters identified within a network can be used to generate candidate disease lists. Examples of disease clusters within a network are described in the examples that follow and are shown, for example, in FIGS. 8A, 8B and 10.

The chemical compound can be any chemical, including, for example natural and food-borne chemical compounds, therapeutic chemicals, such as polyphenols, synthetic drugs, and nutraceuticals, and nontherapeutic chemicals, such as toxins, and general phytochemicals present in food. In the examples that follow, polyphenols are described for illustration purposes only.

The protein-protein interaction network can be, for example, the human interactome, which includes a map of protein interactions in the human cell. Other protein-protein interaction networks can be used, such as, for example, networks from STRINGDB and GeneMania databases.

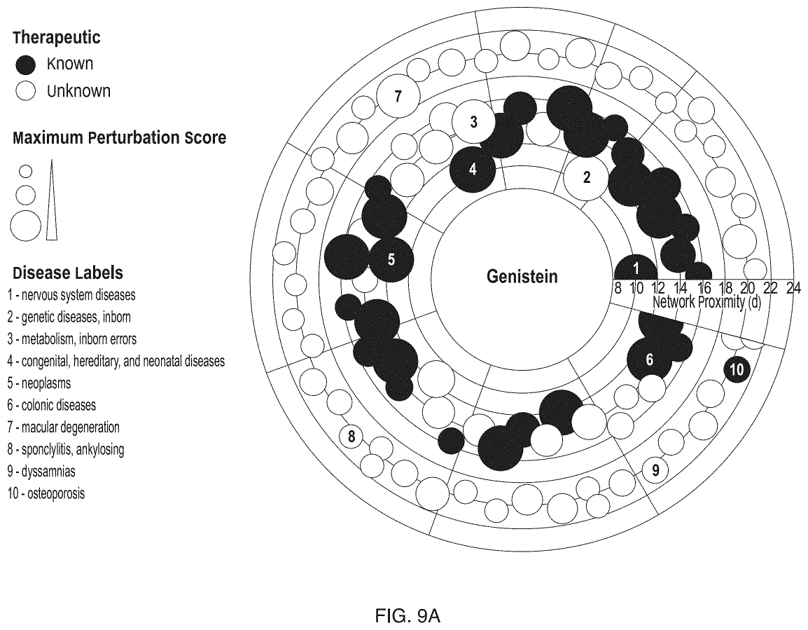

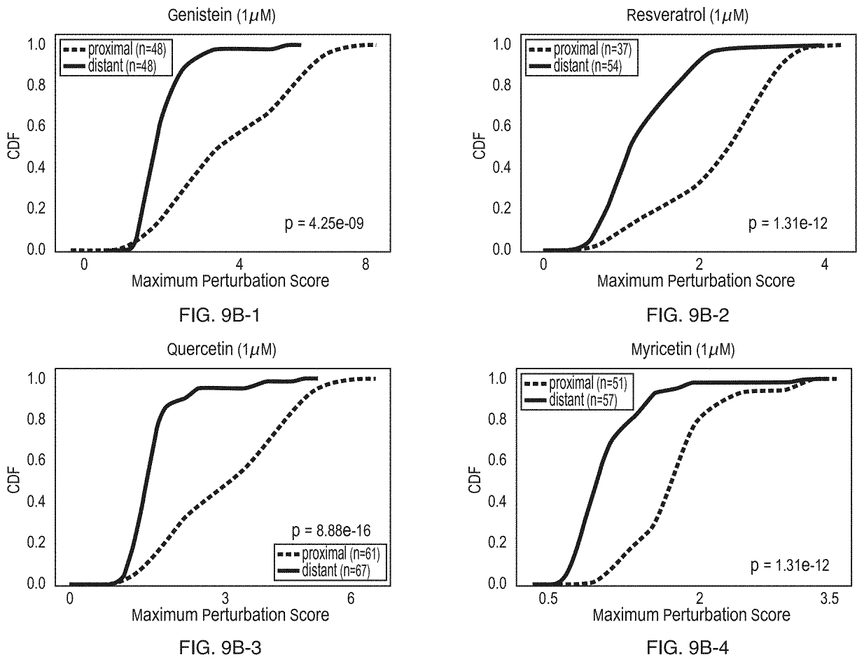

In the systems and methods shown in FIGS. 1 and 2, where several diseases and/or several chemicals are considered, a Chemical-Disease Perturbation Ranking (CDPR) can be produced. The CDPR can provide for identification of chemical compounds that can be used for disease treatment or that present health-related effects, while also providing for mechanistic information of chemical-disease relationships. Examples of disease clusters within a network are described in the examples that follow and are shown, for example, in FIG. 9A.

As further described in the examples that follow, generating the candidate disease list can include generating a proximity value for a disease and the therapeutic chemical. Proximity between a disease and a chemical can be evaluated using a distance metric that takes into account path lengths between chemical targets and disease proteins within the network. For example, the proximity value can be determined based on shortest path lengths between nodes representing proteins associated with the disease and nodes representing proteins associated with the therapeutic chemical. The proximity value can be a distance metric d_{c}(S,T) determined according to:

$${d}_{c} ( S , T ) = \frac{1}{‖ T ‖} {Σ}_{t ∈ T} {min}_{s ∈ S} d ( s , t ) \qquad [ 1 ]$$

where S is a set of proteins associated with the disease, T is a set of proteins associated with the therapeutic chemical, s is a node representing a protein in set S, t is a node representing a protein in set T, and d(s,t) is a shortest path length between nodes s and t in the protein network.

To assess significance of a distance between a chemical and a disease (S,T), a reference distance distribution corresponding to expected distances between two randomly selected groups of proteins matching size and degrees of the original disease proteins and chemical targets in the network can be used. For example, a reference distance distribution can be generated by calculating a proximity between two randomly selected groups, and this procedure can be repeated several (e.g., 100, 500, 1000, 2000) times. The mean and standard deviation of the reference distribution can be used to convert the absolute distance to a relative distance (Z-score). Due to the scale-free nature of the human interactome, there are few nodes with high degrees. To avoid repeatedly choosing the same (high degree) nodes, a degree-preserving random selection can be performed.

As further described in the examples that follow, generating an enrichment score for diseases of a candidate disease list can include measuring an extent of gene expression perturbation by the therapeutic chemical for a given disease. This can include performing a Gene Set Enrichment Analysis. For example, perturbation signatures can be obtained, such as from the ConnectivityMap database, for cell lines treated with different chemicals. These signatures reflect the perturbation of the gene expression profile caused by treatment with a chemical under consideration relative to a reference population, which is composed of other treatments in the same experimental plate. For chemicals having more than one experimental instance (e.g., time of exposure, cell line, dose), the one with highest distil\_cc\_q75 value (i.e., 75th quantile of pairwise spearman correlations in landmark genes) can be selected. Gene Set Enrichment Analysis can then be performed to evaluate the enrichment of disease genes among the top deregulated genes in the perturbation profiles. This analysis results in an Enrichment Score (ES) that has small values when genes are randomly distributed among the ordered list of expression values and high values when genes are concentrated at the top or bottom of the list. Methods of performing an Enrichment Analysis are further described in Subramanian, A. et al. “Gene set enrichment analysis: a knowledge-based approach for interpreting genome-wide expression profiles.” Proc. Natl. Acad. Sci. U.S.A. 102, 15545-50 (2005), the entire contents of which is incorporated herein by reference.

An ES significance can be calculated by creating, for example, 1000 random selections of gene sets with the same size as the original gene set and calculating an empirical p-value by considering a proportion of random sets resulting in ES smaller than the original case. The p-value can be adjusted for multiple testing by using the Benjamini-Hochberg method.

With the proximity values and enrichment scores, the diseases of the candidate disease list can be ranked to provide the CDPR. For example, the ranking can prioritize chemicals by therapeutic potential. The chemicals with greatest therapeutic potential can be defined as those that are proximal to disease proteins and significantly perturb expression of disease genes. The CDPR can advantageously provide for prioritization of a set of chemicals in respect to a disease, or a set of diseases in respect to a chemical, for further evaluation. The CDPR can also provide for a quantitative and molecular-based description of a relationship between chemical compound targets and disease processes, which can in-turn provide for mechanism-of-action information for the chemical compounds.

Conventional methods of evaluating chemical-disease relations involve evaluation of structural properties of chemical compounds. The methods and systems described can advantageously omit such analysis by accounting for how a chemical interacts with various proteins and how those proteins interact with each other and with associated disease processes through the protein-protein interaction network. The methods and systems described do not require knowledge of the specific type of interactions (e.g., activation, inhibition) between a chemical and its protein targets.

In the case of polyphenols, or other food-borne chemicals, the systems and methods described can advantageously provide for the identification of health effects related to chemical compounds present in foods. For example, and as described in the Example sections that follow, from a CDPR, rosmarinic Acid (RA) was shown to have an association with vascular diseases and was predicted to have a direct impact on platelet function. With this information, RA was further evaluated, and experimental evidence demonstrated that RA inhibits platelet aggregation and alpha granule secretion, thereby providing for valuable information of foods that may benefit individuals with poor cardiovascular health.

The systems and methods described can advantageously provide for identification of chemical compounds that can be potentially used for disease treatment, identification of health effects related to chemical compounds, such as those present in foods, and streamlining of research by prioritizing chemicals demonstrated to show bioactivity. This methodology can be coupled with technologies such as CRISPR-CAS9 to genetically change life forms (e.g., plants and their seeds) for greater production of chemical compounds with beneficial health effects.

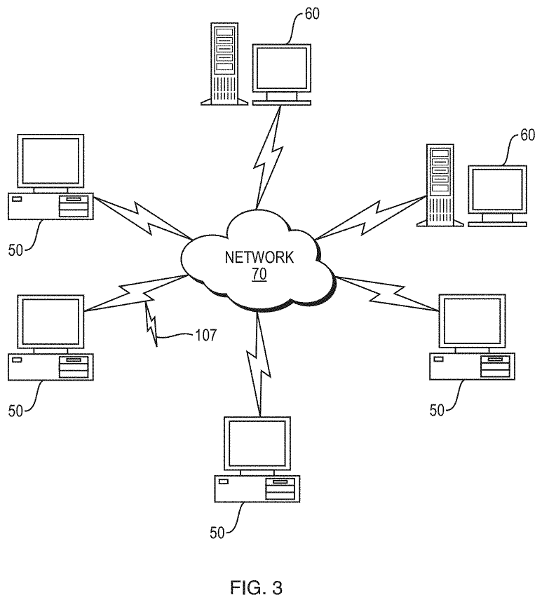

FIG. 3 illustrates a computer network or similar digital processing environment in which the systems and methods described may be implemented. Client computer(s)/devices/exercise apparatuses 50 and server computer(s) 60 provide processing, storage, and input/output devices executing application programs and the like. Client computer(s)/devices 50 can also be linked through communications network 70 to other computing devices, including other client devices/processes 50 and server computer(s) 60. Communications network 70 can be part of a remote access network, a global network (e.g., the Internet), a worldwide collection of computers, cloud computing servers or service, Local area or Wide area networks, and gateways that currently use respective protocols (TCP/IP, Bluetooth, etc.) to communicate with one another. Other electronic device/computer network architectures are suitable.

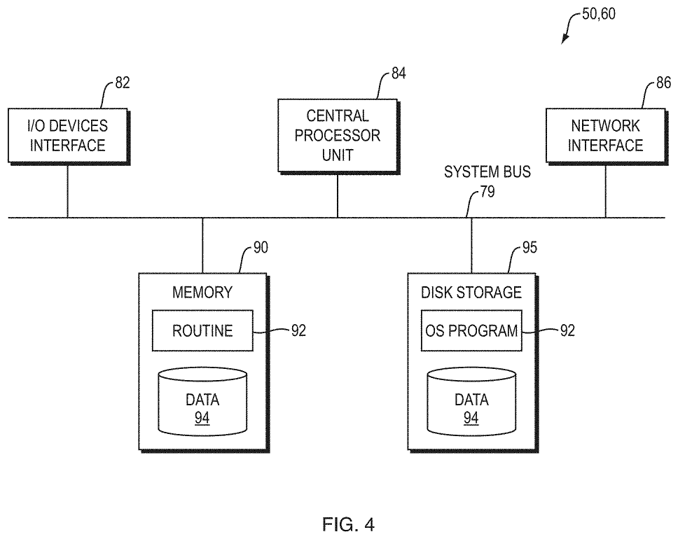

FIG. 4 is a diagram of the internal structure of a computer (e.g., client processor/device 50 or server computers 60) in the computer network of FIG. 3. Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., keyboard, mouse, displays, printers, speakers, etc.) to the computer 50, 60. Network interface 86 allows the computer to connect to various other devices attached to a network (e.g., network 70 of FIG. 3). Memory 90 provides volatile storage for computer software instructions 92 and data 94 used to implement embodiments of the present invention (e.g., processor routines and code for creating a directed acyclic graph (DAG) as a function of computed alignment indices and aligning sequence reads against the DAG being developed, as described herein). Disk storage 95 provides nonvolatile storage for computer software instructions 92 and data 94 used to implement an embodiment of the present invention. Central processor unit 84 is also attached to system bus 79 and provides for the execution of computer instructions.

In particular, embodiments of the present invention execute processor routines for the filter 100 and method 200 of FIGS. 1 and 2, respectively. In one embodiment, the processor routines 92 and data 94 are a computer program product (generally referenced 92), including a non-transitory computer readable medium (e.g., a removable storage medium such as one or more DVD-ROM's, CD-ROM's, diskettes, tapes, etc.) that provides at least a portion of the software instructions for the invention system. Computer program product 92 can be installed by any suitable software installation procedure, as is well known in the art. In another embodiment, at least a portion of the software instructions may also be downloaded over a cable, communication and/or wireless connection. In other embodiments, the invention programs are a computer program propagated signal product 107 embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present invention routines/program 92.

In alternative embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, other mediums and the like.

In other embodiments, the computer program product 92 provides Software as a Service (SaaS) or similar operating platform.

Alternative embodiments can include or employ clusters of computers, parallel processors, or other forms of parallel processing, effectively leading to improved performance, for example, of generating a computational model. Given the foregoing description, one of ordinary skill in the art understands that different portions of processor routine 100 and different iterations operating on respective sequence reads may be executed in parallel on such computer clusters or parallel processors.

The systems and methods described herein were used for the identification of health effects related to polyphenols. For example, the mechanism of action by which rosmarinic acid, a polyphenol present in plants and commonly found in foods, can have a therapeutic effect on cardiovascular diseases was discovered. The mechanism involves the binding of rosmarinic acid to the protein FYN, which results in inhibition of tyrosine phosphorylation in platelets and modulation of different aspects related to platelet function.

The example methodology is described in detail in the Examples herein. In summary, the human interactome, the complete map of known physical interactions among human proteins, was used to identify chemical compounds with a potential effect on vascular diseases (VD), as described herein. This prioritization step yielded several vascular disease or condition associated polyphenols, including rosmarinic acid (RA), as potential modulators of vascular health, and closer inspection of the targets of RA on the human interactome suggested a role in platelet function (VD module).

Accordingly, described herein are methods for treating a vascular disease or condition in a subject (e.g., a subject in need thereof), comprising administering to the subject an effective amount of a vascular disease associated polyphenol, or a pharmaceutically acceptable salt thereof.

Also described herein are methods for promoting or supporting vascular health in a subject (e.g., a subject in need thereof), comprising administering to the subject an effective amount of a vascular disease associated polyphenol, or a pharmaceutically acceptable salt thereof. In some embodiments, the subject has been diagnosed as having a vascular disease or condition, such as any of the vascular diseases or conditions described herein. In some embodiments, the subject has a vascular disease or condition, such as any of the vascular diseases or conditions described herein.

“Treating,” as used herein, refers to taking steps to deliver a therapy to a subject, such as a mammal, in need thereof (e.g., as by administering to a mammal one or more therapeutic agents). “Treating” includes inhibiting the disease or condition (e.g., as by slowing or stopping its progression or causing regression of the disease or condition), and relieving the symptoms resulting from the disease or condition.

“Administering” or “administration” as used herein, refers to taking steps to deliver an agent to a subject, such as a mammal, in need thereof. Administering can be performed, for example, once, a plurality of times, and/or over one or more extended periods. Administration includes both direct administration, including self-administration, and indirect administration, including the act of prescribing a drug or directing a subject to consume an agent. For example, as used herein, one (e.g., a physician) who instructs a subject (e.g., a patient) to self-administer an agent (e.g., a drug), or to have the agent administered by another and/or who provides a patient with a prescription for a drug is administering the agent to the subject.

Non-limiting examples of vascular diseases and conditions treatable in accordance with this disclosure include ischemic injury, diabetes-induced vascular damage, diabetes mellitus, congestive heart failure, coronary heart disease, cerebral ischemia, restenosis after angioplasty, intermittent claudication, myocardial infarction, myocarditis, unstable angina, unstable refractory angina, stable angina, chronic stable angina, acute coronary syndrome, acute myocardial infarction, including first or recurrent myocardial infarction, cardiovascular disease, dyslipidemia, post-prandial lipemia, peripheral vascular disease, renovascular disease, pulmonary hypertension, vasculitis, acute coronary syndromes, modification of cardiovascular risk, modified platelet aggregation, neurodegenerative diseases associated with excess apoptosis (e.g., Parkinson's Disease, Alzheimer's Disease, amyotrophic lateral sclerosis, retinitis pigmentosa, epilepsy), haematologic diseases associated with excess apoptosis (e.g., aplastic anaemia, myelodysplastic syndrome, T CD4+ lymphocytopenia, G6PD deficiency), tissue damage associated with excess apoptosis (e.g., myocardial infarction, cerebrovascular accident, ischemic renal damage, polycystic kidney disease), AIDS, and preeclampsia. Examples of ischemic injuries include injuries caused by cardiovascular ischemia, cerebrovascular ischemia, renal ischemia, hepatic ischemia, ischemic cardiomyopathy, cutaneous ischemia, bowel ischemia, intestinal ischemia, gastric ischemia, pulmonary ischemia, pancreatic ischemia, skeletal muscle ischemia, abdominal muscle ischemia, limb ischemia, ischemic colitis, mesenteric ischemia and silent ischemia.

In some embodiments, the vascular disease or condition is ischemic injury, diabetes-induced vascular damage, diabetes mellitus, congestive heart failure, coronary heart disease, cerebral ischemia, restenosis after angioplasty, intermittent claudication, myocardial infarction, dyslipidemia, post-prandial lipemia, peripheral vascular disease, renovascular disease, pulmonary hypertension, vasculitis, acute coronary syndromes, modification of cardiovascular risk, or modified platelet aggregation. In some embodiments, the vascular disease or condition is coronary heart disease, type 2 diabetes mellitus, cerebral ischemia, or myocardial infarction.

As used herein, “subject” includes humans, domestic animals, such as laboratory animals (e.g., dogs, monkeys, pigs, rats, mice, etc.), household pets (e.g., cats, dogs, rabbits, etc.) and livestock (e.g., pigs, cattle, sheep, goats, horses, etc.), and non-domestic animals. In some embodiments, a subject is a mammal (e.g., a non-human mammal). In some embodiments, a subject is a human.

As used herein, an “effective amount” is an amount sufficient to achieve a desired effect (e.g., therapeutic effect) under the conditions of administration, in vitro, in vivo or ex vivo, such as, for example, an amount sufficient to modulate (e.g., inhibit) platelet function, an amount sufficient to inhibit granule secretion from a platelet, and an amount sufficient to inhibit (e.g., prevent, delay, dampen) a vascular disease or condition (e.g., in a subject). The effectiveness of a therapy can be determined by suitable methods known by those of skill in the art including those described herein.

As used herein, “vascular disease associated polyphenol” refers to a polyphenol identified through CDPR as having an association with a vascular disease. Examples of vascular disease associated polyphenols include pruetin, daidzin, punicalagin, kaempferol 3-o-galactoside, juglone, kaempferol 3-o-glucoside, 4-methylcatechol, rosmarinic acid, xanthotoxin, daidzein, umbelliferone, 1,4-naphthoquinone, 3-caffeoylquinic acid, isoliquiritigenin, chrysin, cinnamic acid, caffeic acid, genistein, 3-phenylpropionic acid, butein, myricetin, piceatannol, piceatannol, ellagic acid, (−)-epigallocatechin 3-o-gallate, phenol, and quercetin.

In an embodiment, the vascular disease associated polyphenol is quercetin, (−)-epicatechin-3-o-gallate, (−)-epigallocatechin-3-o-gallate, myricetin, butein, phenol, 3-phenylpropionic acid, quercetin 3-o-glucoside, apigenin, chrysin, piceatannol, isoliquiritigenin, caffeic acid, 3-caffeoylquinic acid, genistein, cinnamic acid, (−)-epicatechin, kaempeferol, resveratrol, luteolin, or ellagic acid, or a pharmaceutically acceptable salt thereof. In yet another embodiment, the vascular disease associated polyphenol is pruetin, daidzin, punicalagin, kaempferol 3-o-galactoside, juglone, kaempferol 3-o-glucoside, 4-methylcatechol, rosmarinic acid, xanthotoxin, daidzein, umbelliferone, 1,4-naphthoquinone, 3-caffeoylquinic acid, isoliquiritigenin, chrysin, cinnamic acid, caffeic acid, genistein, 3-phenylpropionic acid, butein, myricetin, piceatannol, piceatannol, ellagic acid, (−)-epigallocatechin 3-o-gallate, phenol, or quercetin, or a pharmaceutically acceptable salt thereof. In still a further embodiment, the vascular disease associated polyphenol is gallic acid, 1,4-naphthoquinone, or rosmarinic acid, or a pharmaceutically acceptable salt thereof. In a further embodiment, the vascular disease associate polyphenol is rosmarinic acid, or a pharmaceutically acceptable salt thereof.

The polyphenols described herein, including vascular disease associated polyphenols, can be provided in free base form or in salt form (e.g., pharmaceutically acceptable salt form). Pharmaceutically acceptable salts include acid addition salts and base addition salts. The term “pharmaceutically acceptable salts” embraces salts commonly used to form alkali metal salts and to form addition salts of free acids or free bases. The nature of the salt is not critical, provided that it is pharmaceutically acceptable.

As used herein, the term “pharmaceutically acceptable” refers to species which are, within the scope of sound medical judgment, suitable for use in contact with the tissues of mammals without undue toxicity, irritation, allergic response and the like, and are commensurate with a reasonable benefit/risk ratio. For example, a substance is pharmaceutically acceptable when it is suitable for use in contact with cells, tissues or organs of animals or humans without excessive toxicity, irritation, allergic response, immunogenicity or other adverse reactions, in the amount used in the dosage form according to the dosing schedule, and commensurate with a reasonable benefit/risk ratio.

Suitable pharmaceutically acceptable acid addition salts may be prepared from an inorganic acid or an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, examples of which are formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. Pharmaceutically acceptable acidic/anionic salts also include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts.

Suitable pharmaceutically acceptable base addition salts include, but are not limited to, metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine. Pharmaceutically acceptable basic/cationic salts also include, the diethanolamine, ammonium, ethanolamine, piperazine and triethanolamine salts.

All of these salts may be prepared by conventional means by treating, for example, a polyphenol described herein with an appropriate acid or base.

Polyphenols for use in the methods described herein are conveniently provided for administration (e.g., consumption) in the form of a composition, e.g., a dietary supplement, pharmaceutical composition, or medical food. Compositions can also be in the form of a complete nutritional food, drink, mineral water, soup, food supplement, replacement food, solution, spray, powder, tablet, capsule, nutritional bar, liquid suspension, confectionary, child or infant formulation, tea, tea bag, culinary product, or pet food.

In some embodiments, the composition is in the form of a pharmaceutical composition comprising a polyphenol described herein (e.g., vascular disease associated polyphenol) and a pharmaceutically acceptable carrier.

“Pharmaceutically acceptable carrier” refers to a carrier or excipient that does not destroy the pharmacological activity of the agent with which it is formulated and is, within the scope of sound medical judgment, suitable for use in contact with the tissues of mammals without undue toxicity, irritation, allergic response and the like, and is commensurate with a reasonable benefit/risk ratio. Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For preparing pharmaceutical compositions, pharmaceutically acceptable carriers can either be solid or liquid. Solid form preparations include powders, tablets, pills, capsules, cachets, suppositories, and dispersible granules. For example, the pharmaceutical compositions of the present invention may be in powder form for reconstitution at the time of delivery. A solid carrier can be one or more substances which may also act as diluents, flavoring agents, solubilizers, lubricants, suspending agents, binders, preservatives, tablet disintegrating agents, or an encapsulating material. In powders, the carrier is a finely divided solid which is in a mixture with the finely divided active ingredient.

In tablets, the active ingredient is mixed with the carrier having the necessary binding properties in suitable proportions and compacted in the shape and size desired.

The powders and tablets preferably contain from about one to about seventy percent of the active ingredient. Suitable carriers are magnesium carbonate, magnesium stearate, talc, sugar, lactose, pectin, dextrin, starch, gelatin, tragacanth, methylcellulose, sodium caboxymethylcellulose, a low-melting wax, cocoa butter, and the like. Tablets, powders, cachets, lozenges, fast-melt strips, capsules and pills can be used as solid dosage forms containing the active ingredient suitable for oral administration.

Liquid form preparations include solutions, suspensions, retention enemas, and emulsions, for example, water or water propylene glycol solutions. For parenteral injection, liquid preparations can be formulated in solution in aqueous polyethylene glycol solution.

Aqueous solutions suitable for oral administration can be prepared by dissolving the active ingredient in water and adding suitable colorants, flavors, stabilizing agents, and thickening agents as desired. Aqueous suspensions for oral administration can be prepared by dispersing the finely divided active ingredient in water with viscous material, such as natural or synthetic gums, resins, methylcellulose, sodium carboxymethylcellulose, and other well-known suspending agents.

The composition is preferably in unit dosage form. In such form, the composition is subdivided into unit doses containing appropriate quantities of the active ingredient. The unit dosage form can be a packaged preparation, the package containing discrete quantities of, for example, tablets, powders, and capsules in vials or ampules. Also, the unit dosage form can be a tablet, cachet, capsule, or lozenge itself, or it can be the appropriate amount of any of these in packaged form. The composition may be a food composition in the form of complete nutritional foods, drinks, mineral waters, soups, food supplements and replacement foods, solutions, sprays, powders, tablets, capsules, nutritional bars, liquid bacterial suspensions, confectionary, milk-based or fermented-milk based products, yogurts, milk-based powders, nutrition products, compositions for children and/or infants, cereal-based products, ice creams, chocolate, coffee, tea, or pet food. The quantity of active ingredient in a unit dose preparation may be varied or adjusted from about 0.1 mg to about 1000 mg, preferably from about 0.1 mg to about 100 mg (e.g., for intravenous administration) or from about 1.0 mg to about 1000 mg (e.g., for oral administration) or from about 1.0 g to about 10.0 g (e.g., for food composition). The dosages, however, may be varied depending, for example, upon the requirements of the patient, the severity of the condition being treated, the composition and the route of administration being employed. Determination of the proper dosage for a particular situation is within the skill in the art. Also, the composition may contain, if desired, other compatible therapeutic agents.

Compositions described herein and, hence, polyphenols in the compositions can be administered orally, parenterally (including subcutaneously, intramuscularly, intravenously and intradermally), by inhalation spray, topically, rectally, nasally, buccally, vaginally or via an implanted reservoir. In some embodiments, provided compounds or compositions are administrable intravenously and/or intraperitoneally. The term “parenteral,” as used herein, includes subcutaneous, intracutaneous, intravenous, intramuscular, intraocular, intravitreal, intra-articular, intra-arterial, intra-synovial, intrasternal, intrathecal, intralesional, intrahepatic, intraperitoneal intralesional and intracranial injection or infusion techniques. Preferably, the compositions are administrable orally.

Thus, in some embodiments of the methods described herein, a polyphenol or a composition comprising a polyphenol is administered by injection, intravenously, intraarterially, intraocularly, intravitreally, subdermally, orally, buccally, nasally, transmucosally, topically, in an ophthalmic preparation, or by inhalation. In some embodiments, a polyphenol or a composition comprising a polyphenol is administered orally.

The compositions disclosed herein are prepared in accordance with standard procedures and are administered at dosages that are selected to reduce, prevent, or eliminate, or to slow or halt the progression of, the condition being treated (See, e.g., Remington's Pharmaceutical Sciences, Mack Publishing Company, Easton, PA, and Goodman and Gilman's The Pharmaceutical Basis of Therapeutics, McGraw-Hill, New York, N.Y., the contents of which are incorporated herein by reference, for a general description of the methods for administering various agents for human therapy). The compositions can be delivered using controlled or sustained-release delivery systems (e.g., capsules, biodegradable matrices). Exemplary delayed-release delivery systems for drug delivery that would be suitable for administration of a composition described herein are described in U.S. Pat. No. 5,990,092 (issued to Walsh); U.S. Pat. No. 5,039,660 (issued to Leonard); U.S. Pat. No. 4,452,775 (issued to Kent); and U.S. Pat. No. 3,854,480 (issued to Zaffaroni), the entire teachings of which are incorporated herein by reference.

For oral administration, the compositions may be in the form of, for example, a tablet, capsule, suspension or liquid. The composition is preferably made in the form of a dosage unit containing a therapeutically effective amount of the active ingredient. Examples of such dosage units are tablets and capsules. For therapeutic purposes, the tablets and capsules can contain, in addition to the active ingredient, conventional carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica, or talc; disintegrants, for example potato starch, flavoring or coloring agents, or acceptable wetting agents. Oral liquid preparations generally in the form of aqueous or oily solutions, suspensions, emulsions, syrups or elixirs may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

For topical use the invention may also be prepared in suitable forms to be applied to the skin, or mucus membranes of the nose and throat, and may take the form of creams, ointments, liquid sprays or inhalants, lozenges, or throat paints. Such topical formulations further can include chemical compounds such as dimethylsulfoxide (DMSO) to facilitate surface penetration of the active ingredient. Suitable carriers for topical administration include oil-in-water or water-in-oil emulsions using mineral oils, petrolatum and the like, as well as gels such as hydrogel. Alternative topical formulations include shampoo preparations, oral pastes and mouthwash.

For application to the eyes or ears, the compositions of the present invention may be presented in liquid or semi-liquid form formulated in hydrophobic or hydrophilic bases as ointments, creams, lotions, paints or powders.

For rectal administration the compositions of the present invention may be administered in the form of suppositories admixed with conventional carriers such as cocoa butter, wax or other glyceride. For preparing suppositories, a low-melting wax, such as a mixture of fatty acid glycerides or cocoa butter, is first-melted and the active ingredient is dispersed homogeneously therein, as by stirring. The molten homogeneous mixture is then poured into convenient sized molds, allowed to cool, and thereby to solidify.

Delivery can also be by injection into the brain or body cavity of a patient or by use of a timed release or sustained release matrix delivery systems, or by onsite delivery using micelles, gels and liposomes. Nebulizing devices, powder inhalers, and aerosolized solutions are representative of methods that may be used to administer such preparations to the respiratory tract. Delivery can be in vitro, in vivo, or ex vivo.

For example, suitable intravenous dosages for the invention can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment.

A desired dose may conveniently be administered in a single dose, for example, such that the agent is administered once per day, or as multiple doses administered at appropriate intervals, for example, such that the agent is administered 2, 3, 4, 5, 6 or more times per day. The daily dose can be divided, especially when relatively large amounts are administered, or as deemed appropriate, into several, for example 2, 3, 4, 5, 6 or more, administrations. Typically, the compositions will be administered from about 1 to about 6 (e.g., 1, 2, 3, 4, 5 or 6) times per day or, alternatively, as an infusion (e.g., a continuous infusion).

Determining the dosage and route of administration for a particular agent, patient and vascular disease or condition is well within the abilities of one of skill in the art. Preferably, the dosage does not cause or produces minimal adverse side effects.

Doses lower or higher than those recited above may be required. Specific dosage and treatment regimens for any particular subject will depend upon a variety of factors, for example, the activity of the specific agent employed, the age, body weight, general health status, sex, diet, time of administration, rate of excretion, drug combination, the severity and course of the disease, condition or symptoms, the subject's disposition to the disease, condition or symptoms, the judgment of the treating physician and the severity of the particular disease being treated. The amount of an agent in a composition will also depend upon the particular agent in the composition.

In some embodiments, the concentration of one or more active agents provided in a composition is less than 100%, 90%, 80%, 70%, 60%, 50%, 40%, 30%, 20%, 19%, 18%, 17%, 16%, 15%,14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, or 0.01% w/w, w/v or v/v; and/or greater than 90%, 80%, 70%, 60%, 50%, 40%, 30%, 20%, 10%, 5%, 1%, 0.5%, 0.4%, 0.3%, 0.2%, 0.1%, or 0.01% w/w, w/v, or v/v.

In some embodiments, the concentration of one or more active agents provided in a composition is in the range from about 0.01% to about 50%, about 0.01% to about 40%, about 0.01% to about 30%, about 0.05% to about 25%, about 0.1% to about 20%, about 0.15% to about 15%, or about 1% to about 10% w/w, w/v or v/v. In some embodiments, the concentration of one or more active agents provided in a composition is in the range from about 0.001% to about 10%, about 0.01% to about 5%, about 0.05% to about 2.5%, or about 0.1% to about 1% w/w, w/v or v/v.

Also provided herein are methods of modulating (e.g., inhibiting) platelet function, comprising contacting platelets with a vascular disease associated polyphenol, or a pharmaceutically acceptable salt thereof. The methods may be conducted in vitro, in vivo (e.g., in a subject, such as a subject in need thereof) or ex vivo. Thus, some embodiments provide a method of modulating (e.g., inhibiting) platelet function in a subject (e.g., a subject in need thereof), comprising administering to the subject an effective amount of a vascular disease associated polyphenol, or a pharmaceutically acceptable salt thereof.

In some embodiments, the platelet function is platelet aggregation. In some embodiments, the platelet function is granule secretion (e.g., alpha-granule secretion, dense granule secretion).

In some embodiments (e.g., in vitro embodiments), the concentration of vascular disease associated polyphenol, or a pharmaceutically acceptable salt thereof, is from about 1 μM to about 10 mM, for example, from about 10 μM to about 1 mM, at least about 10 μM at least about 20 μM at least about 50 μM at least about 100 μM at least about 200 μM or at least about 500 μM. In some embodiments, platelet function is reduced by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% compared to platelet function of platelets not contacted with (e.g., by administration) the vascular disease associated polyphenol, or a pharmaceutically acceptable salt thereof.

### EXEMPLIFICATION

### Example 1: Predicting Health Impact of Dietary Polyphenols Using a Chemical-Disease Perturbation Ranking

Despite the widespread evidence of the positive role of polyphenols on human health, the underlying molecular mechanisms through which specific polyphenols exert their function remain largely unexplored. From a mechanistic perspective their role is rather special because dietary polyphenols are not processed by the endogenous metabolic processes of anabolism and catabolism. Rather, dietary polyphenols impact human health through their ant- or pro-oxidant activity, by binding to proteins and modulating the activity of key cellular signaling and metabolic pathways, interacting with digestive enzymes, and modulating gut microbiota growth. Yet, the variety of experimental settings used so far to explore the molecular effects of polyphenols—represented by different concentrations, administration routes, model organisms, populations, and evaluated outcomes—have, to date, offered a range of often conflicting evidence for interpretation. For example, different clinical trials resulted in contrasting conclusions about the beneficial effects of resveratrol on glycemic control of type 2 diabetes patients. Therefore, there is a need for a framework to interpret the evidence present in the literature, and to offer in-depth mechanistic predictions on the molecular pathways responsible for the health implications of polyphenols present in diet. These insights can aid in the development of novel diagnostic and therapeutic strategies, and may lead to the synthesis of novel drugs.

A network medicine framework was developed to capture the molecular interactions between polyphenols and their cellular binding targets, unveiling their relationship to complex diseases. The developed framework is based on the human interactome, a comprehensive network of all known physical interactions between human proteins, which has been validated before as a platform for understanding disease mechanisms, rational drug target identification, and drug repurposing.

First, it was found that the proteins to which polyphenols bind form identifiable neighborhoods in the human interactome. It was then demonstrated that the proximity between polyphenol targets and proteins associated with specific diseases is predictive of the known therapeutic effects of polyphenols. Finally, the potential therapeutic effects of rosmarinic acid on vascular diseases was unveiled with a prediction that the effect was related to modulation of platelet function. This prediction was confirmed by the performance of experiments that demonstrated that rosmarinic acid modulates platelet function in vitro by inhibiting tyrosine protein phosphorylation. Altogether, the results demonstrate that the network-based relationship between disease proteins and polyphenol targets offers a tool to systematically unveil the health effects of polyphenols.

The methodology described can provide for the foundation of mechanistic interpretation of alternative pathways through which polyphenols can affect health: e.g., the combined effect of different polyphenols and their interaction with drugs. Furthermore, the methodology described can be applied to other food-related chemicals, providing a framework to understand their health effects.

### Example 2: Results: Polyphenol Targets Cluster in Specific Functional Neighborhoods of the Interactome

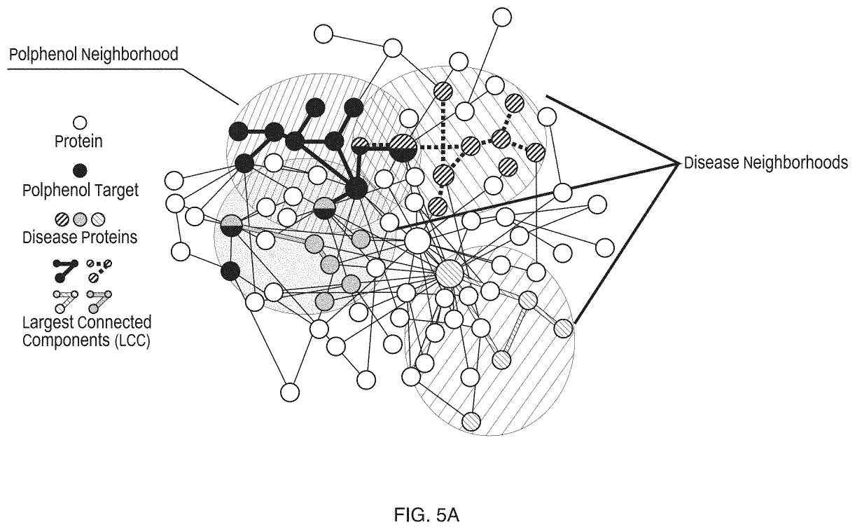

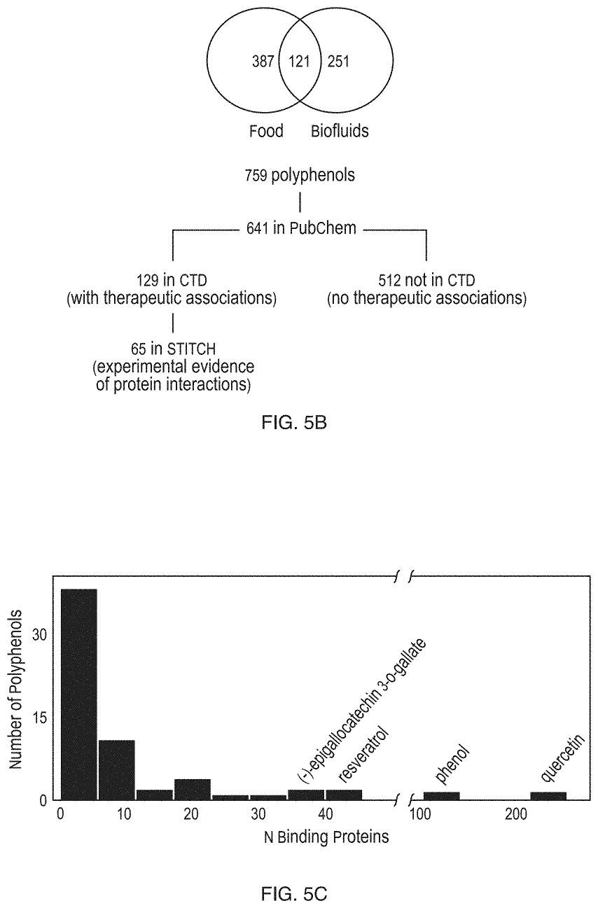

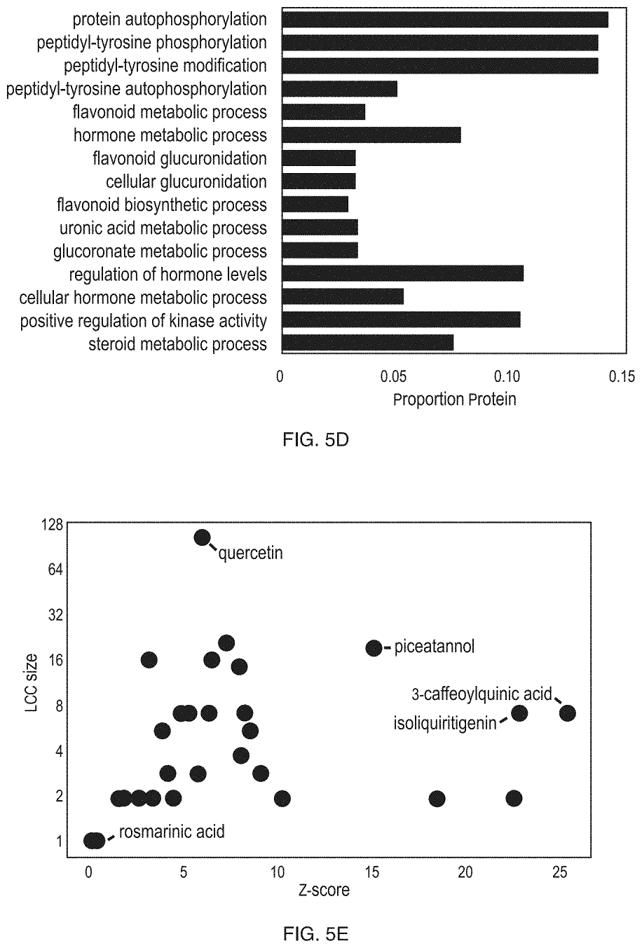

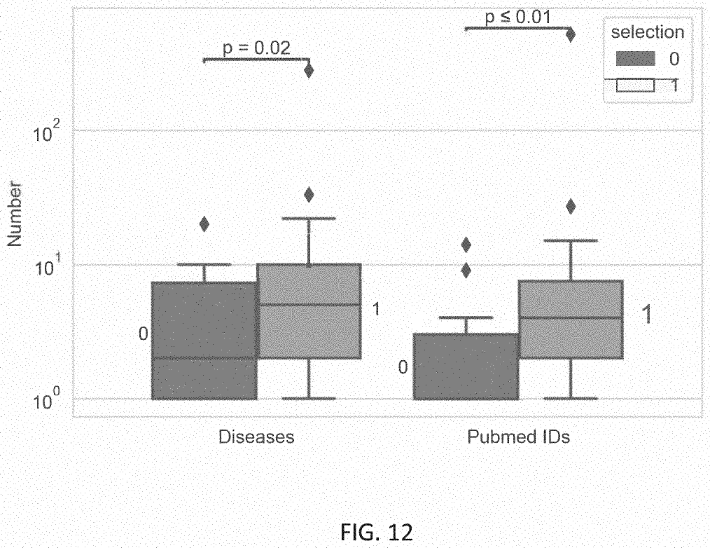

The study started with a list of 759 polyphenols catalogued in the PhenolExplorer database, of which 387 were only detected in foods, 251 were only detected in biofluids, and 121 are present in both foods and biofluids (FIG. 5B). From the list, 118 (15%) polyphenols were removed for which PubChem IDs could not be identified and 512 (67%) that lacked a manually curated ‘therapeutic’ label in the Comparative Toxicogenomics Database (CTD). Of the remaining 129 polyphenols, 65 have experimentally validated protein targets in the STITCH database (Table 4), providing for the group of polyphenols that were the center of the study. This group represented well-studied polyphenols, from EGCG, the active ingredient of green tea with demonstrated glucose lowering properties, to polyphenols that have the largest number disease associations in CTD (FIG. 12). Of these 14 were detected in blood according to the Human Metabolome Database, with maximum concentrations ranging from 10 nM to 80 μM (Table 4), and, of the remaining 51, 35 were predicted to have high gastrointestinal absorption (Tables 4 and 5).

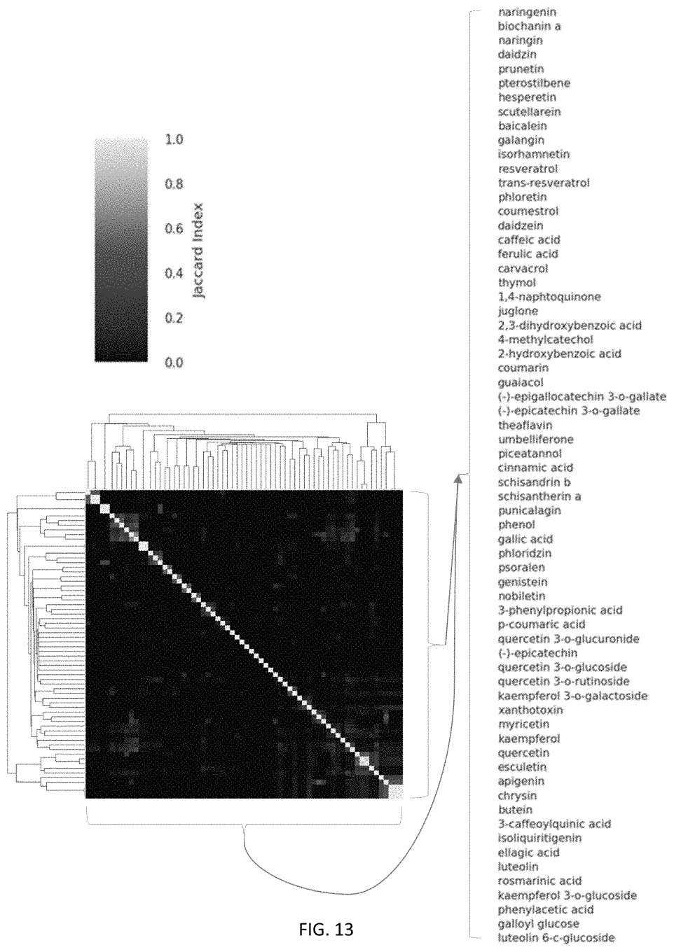

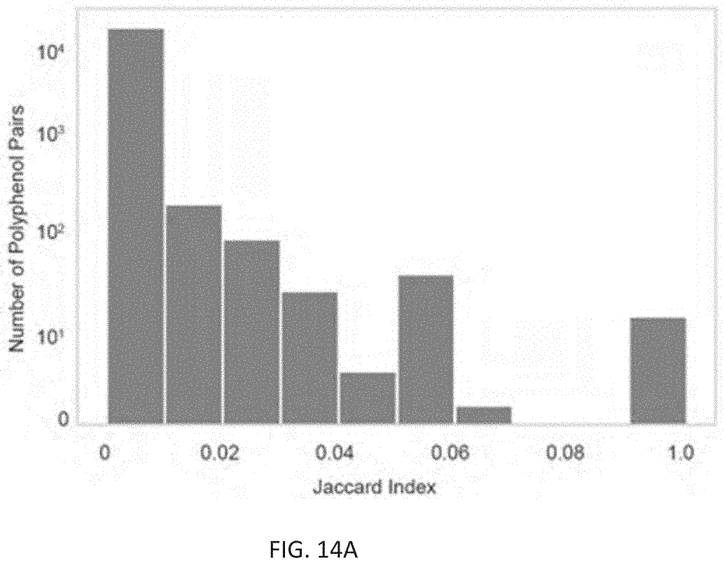

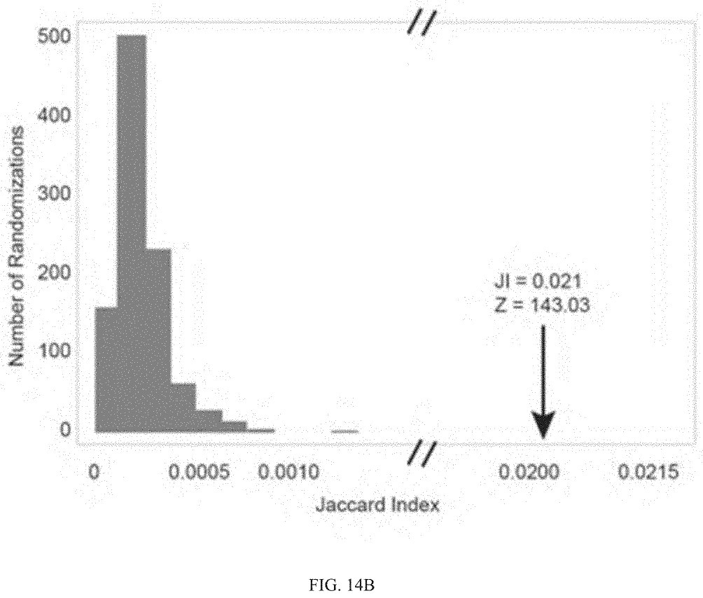

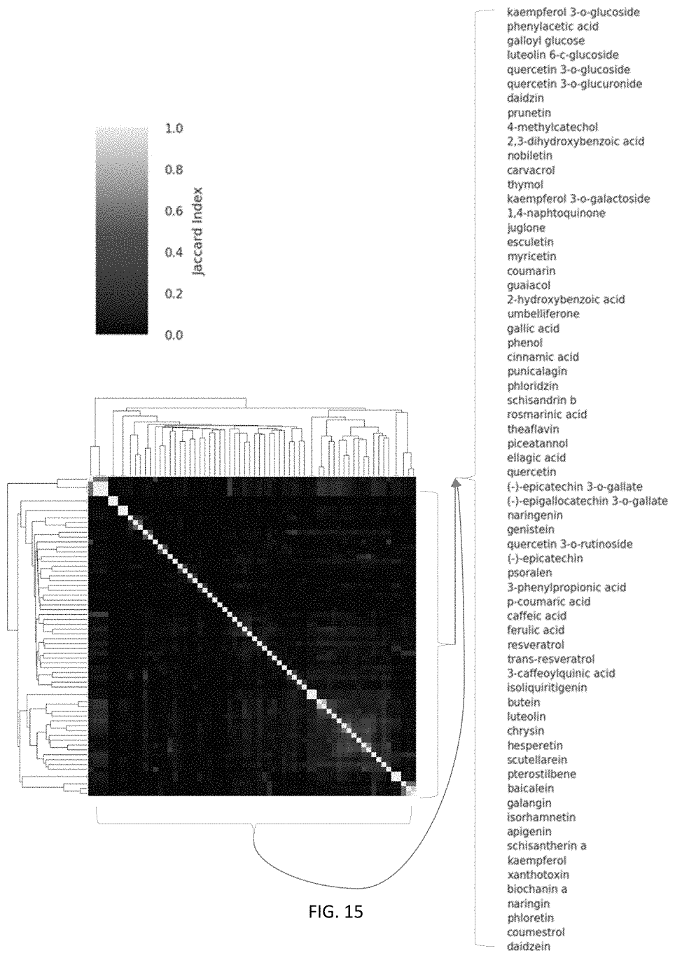

To identify the cellular processes potentially affected by specific polyphenol molecules, the polyphenol targets were mapped to the human interactome, consisting of 17,651 proteins and 351,393 interactions (FIG. 5A). It was found that 19 of the 65 studied polyphenols have only one protein target, while a few polyphenols have an exceptional number of recorded targets, like quercetin (216 targets), phenol (98), resveratrol (63), (−)-epigallocatechin 3-o-gallate (51), and ellagic acid (42) (FIG. 5C). The Jaccard Index (JI) of the protein targets of each polyphenol pair was computed, and only a limited similarity of targets among different polyphenols (average JI=0.0206) (FIG. 13 and FIG. 14A) was found. Even though the average JI was small, it was still significantly higher (Z=147, FIG. 14B) than the JI expected if the polyphenol targets were randomly assigned from the pool of all network proteins with degrees matching the original set. This finding suggests that while each polyphenol targets a specific set of proteins, their targets are confined to a common pool of proteins, likely determined by commonalities in the binding domains the three-dimensional structure of the protein targets. Gene Ontology (GO) Enrichment Analysis of all polyphenol protein targets revealed that they tend to target pathways related to post-translation protein modifications, regulation, and xenobiotic metabolism (FIG. 5D and FIG. 15). The enriched GO categories indicate that polyphenols modulate common regulatory processes, but the low similarity in their protein targets, illustrated by the low average JI, indicates that they target different processes within the same pathways.

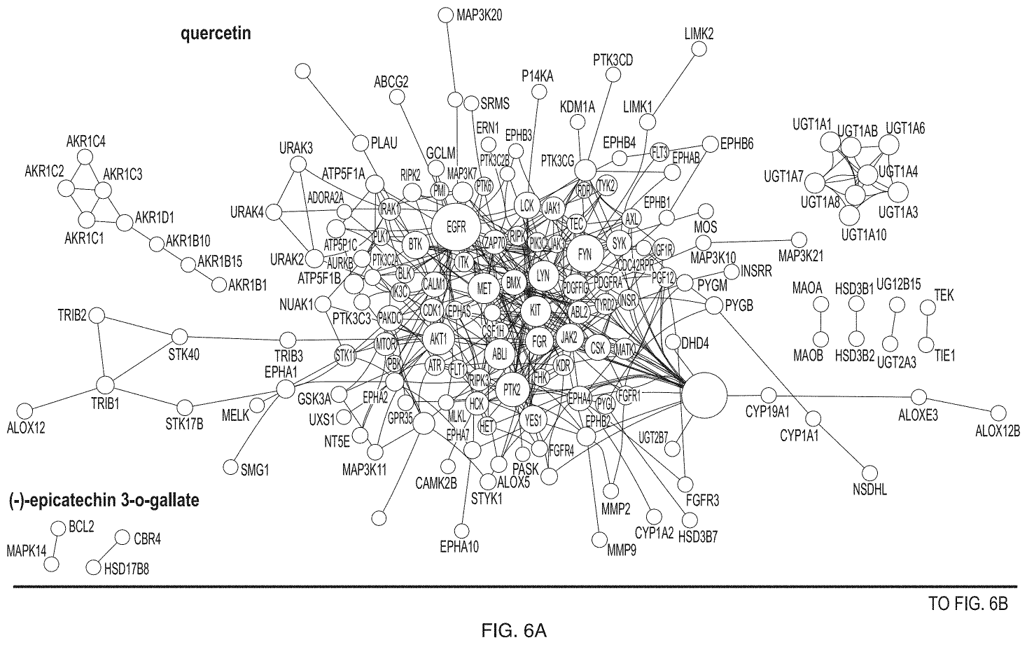

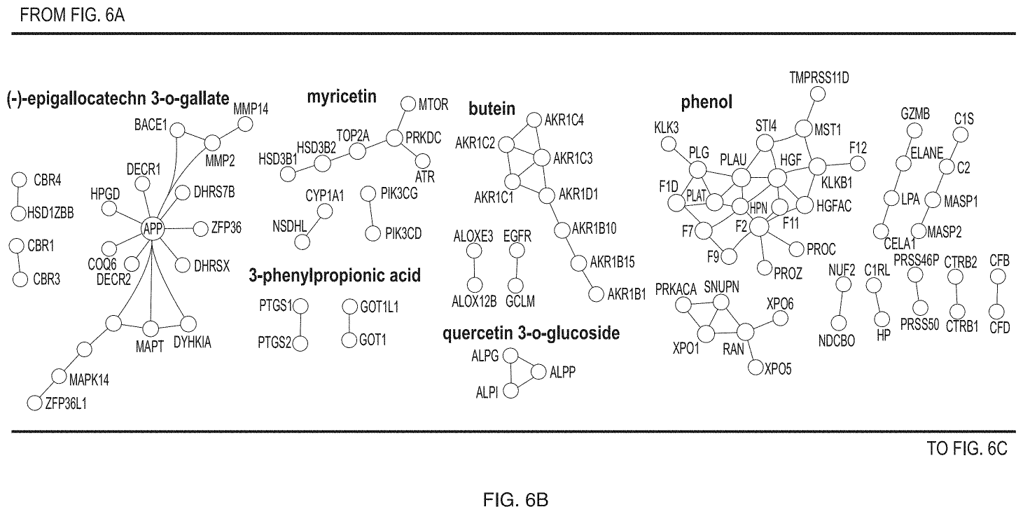

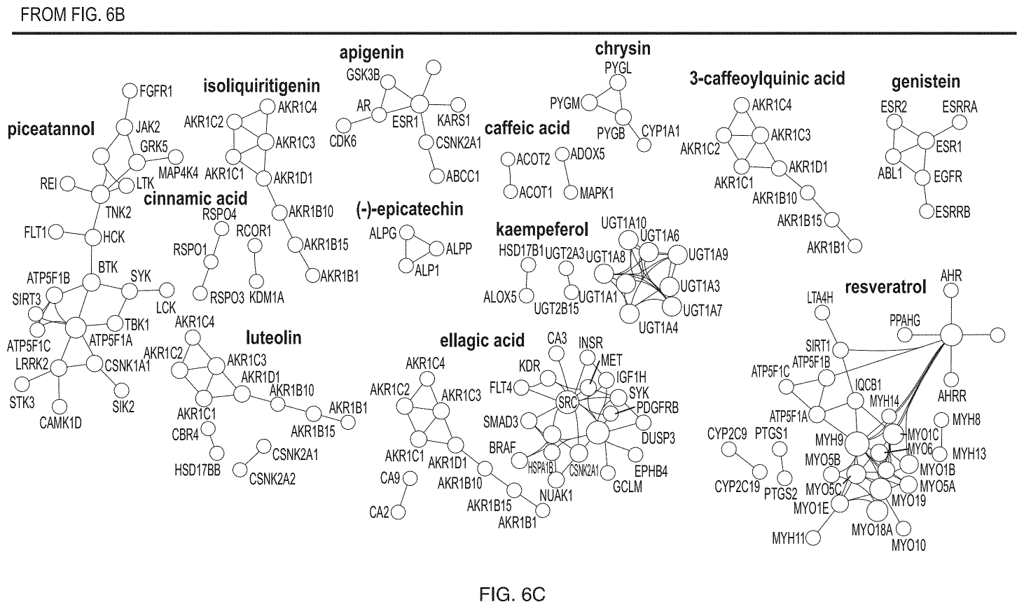

It was next asked whether the polyphenol targets cluster in specific regions of the human interactome. The focus was on polyphenols with more than two targets (n=46, FIGS. 6A-6C) and measured the size and significance of the largest connected component (LCC) formed by the targets of each polyphenol. It was found that 25 of the 46 polyphenols have a larger LCC than expected by chance (Z-score>1.95) (FIG. 5E, FIGS. 6A-6C). In agreement with experimental evidence documenting the effect of polyphenols on multiple pathways, it was found that ten polyphenols have their targets organized in multiple connected components of size >2. For example, the phenol targets, a compound with antiseptic and disinfectant properties, form three connected components with sizes 19, 6, 4 and 5 components of size 2 (FIGS. 6A-6C).

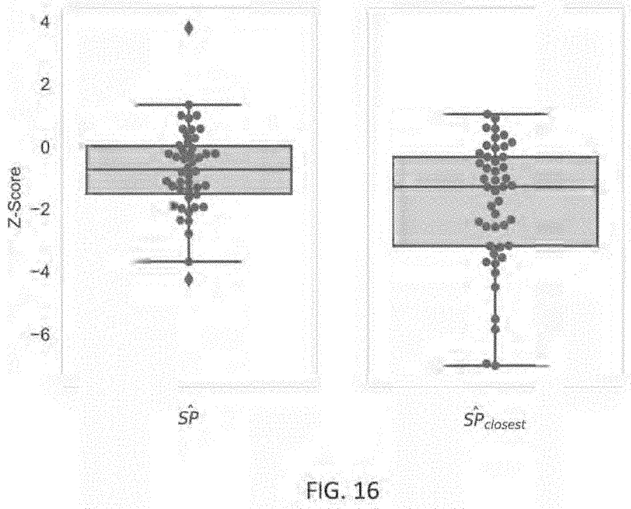

Taken together, these results indicate that the targets of polyphenols modulate specific well localized neighborhoods of the interactome (FIGS. 6A-6C, FIG. 16).

### Example 3: Proximity Between Polyphenol Targets and Disease Proteins Reveals their Therapeutic Effects

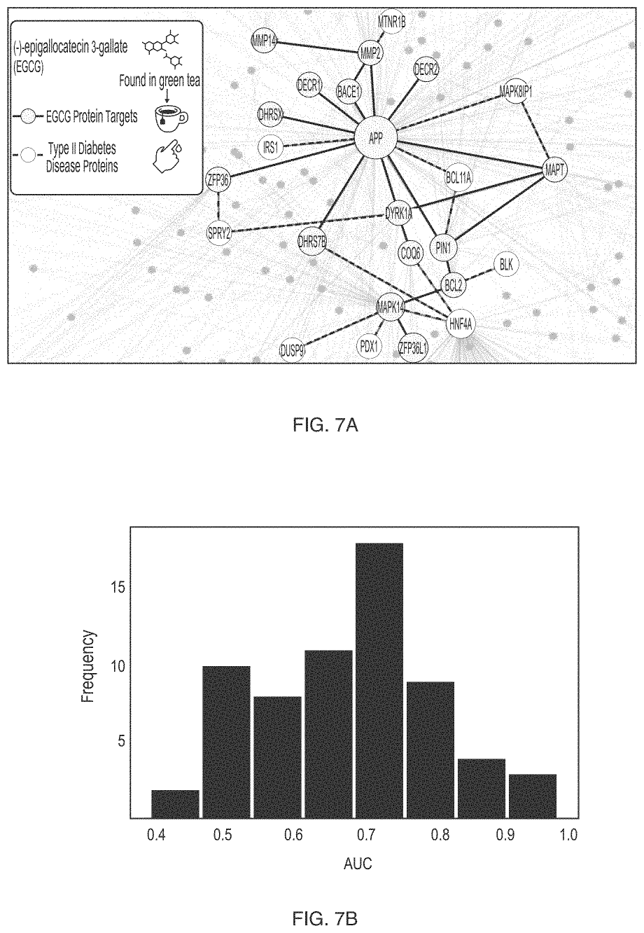

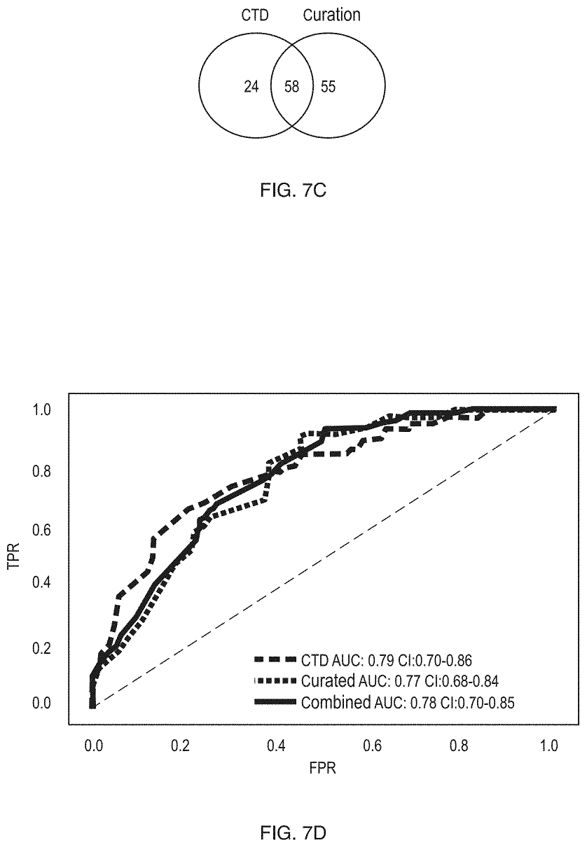

Polyphenols act like drugs: they bind to specific proteins, affecting their ability to perform their normal functions. The closer the targets of a polyphenol are to disease proteins, the more likely that the polyphenol will affect the disease phenotype, resulting in detectable therapeutic effects on the disease. The network proximity between polyphenol targets and proteins associated with 299 diseases was calculated using the closest measure, d_{c}, representing the average shortest path length between each polyphenol target and the nearest disease protein. Consider for example (−)-epigallocatechin 3-O-gallate (EGCG), a polyphenol abundant in green tea. Epidemiological studies have found a positive relationship between green tea consumption and reduced risk of type 2 diabetes mellitus (T2D), and physiological and biochemical studies have shown that EGCG presents glucose-lowering effects in both in vitro and in vivo models. Fifty-four experimentally validated EGCG protein targets were identified and mapped to the interactome, and it was found that the ECGC targets form an LCC of 17 proteins (Z=7.61) (FIG. 7A). The network-based distance between EGCG targets and 83 proteins associated with T2D was also computed, and it was found that the two sets are significantly proximal to each other. Indeed, several T2D proteins directly interact with the protein targets within the EGCG LCC (FIG. 7A). All 299 diseases were ranked based on the network proximity to the ECGC targets to determine if the 82 diseases in which ECGC has known therapeutic effects according to the CTD database could be recovered. The list recovered 15 previously known therapeutic associations among the top 20 ranked diseases (Table 1), confirming that network-proximity can discriminate between known and unknown disease associations for polyphenols, as previously confirmed in drugs. It was therefore demonstrated that the network proximity methods can be used to unveil novel therapeutic associations between food chemicals and diseases.

These methods were expanded to all polyphenol-disease pairs, with the goal of predicting diseases for which specific polyphenols might have therapeutic effects. For this, all possible 19,435 polyphenol-disease associations between 65 polyphenols and 299 diseases were grouped into known (1,525) and unknown (17,910) associations. The known polyphenol-disease set was retrieved from CTD, limiting to manually curated associations for which there is literature-based evidence. For each polyphenol, how well network proximity discriminates between the known and unknown sets was tested by evaluating the area under the Receiving Operating Characteristic (ROC) curve (AUC). For EGCG, network proximity offers a good discriminative power (AUC=0.78, CI: 0.70-0.86) between diseases with known and unknown therapeutic associations (Table 1). It was found that network proximity (d_{c}) offers predictive power with an AUC>0.7 for 31 polyphenols (FIG. 7B). In Table 2 the top 10 polyphenols for which the network medicine framework offered the best predictive power of therapeutic effects are summarized, the entries limited to prediction performance of AUC>0.6 and performance over top predictions with Precision>0.6.

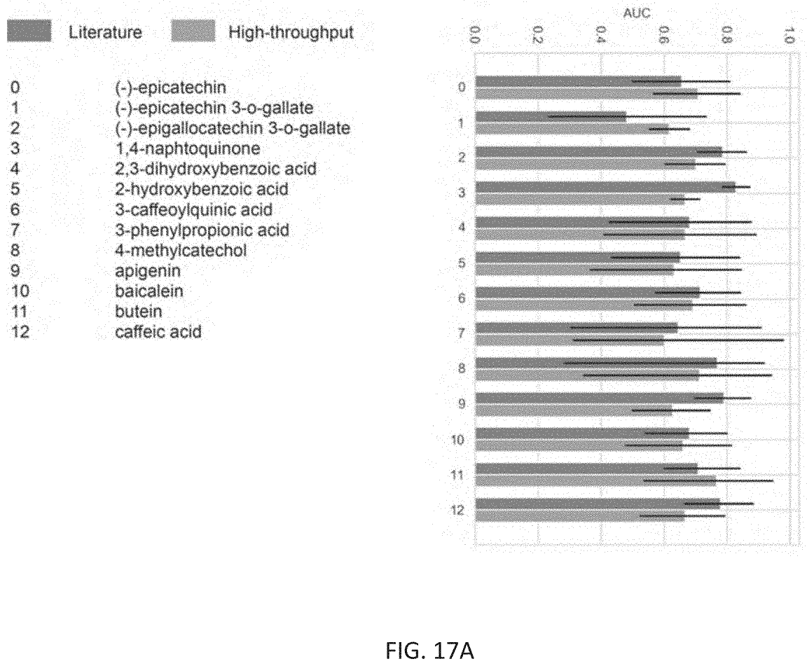

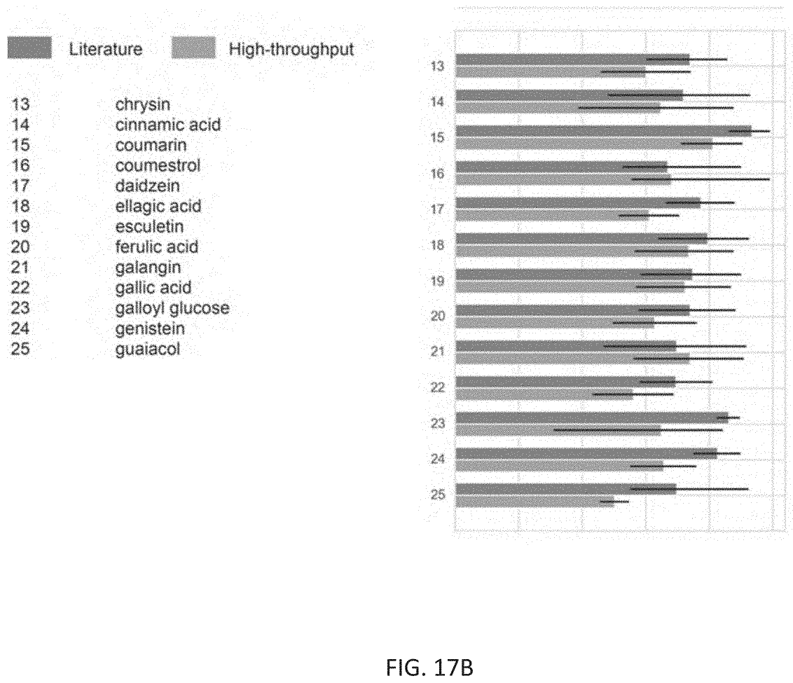

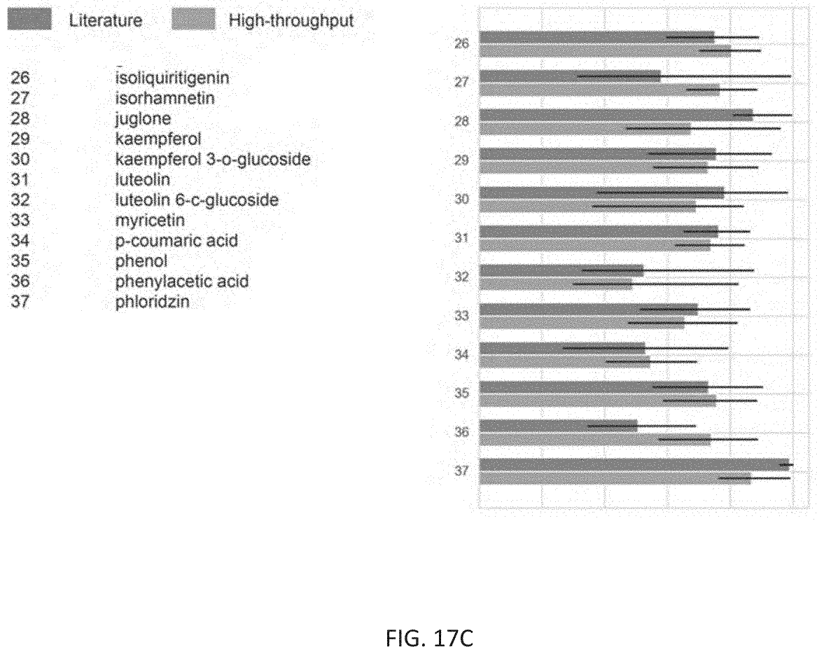

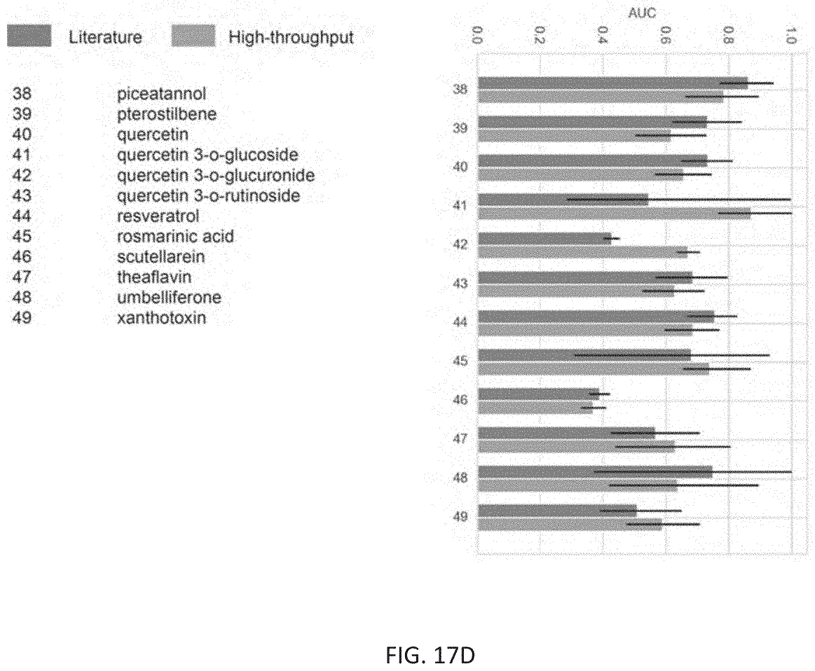

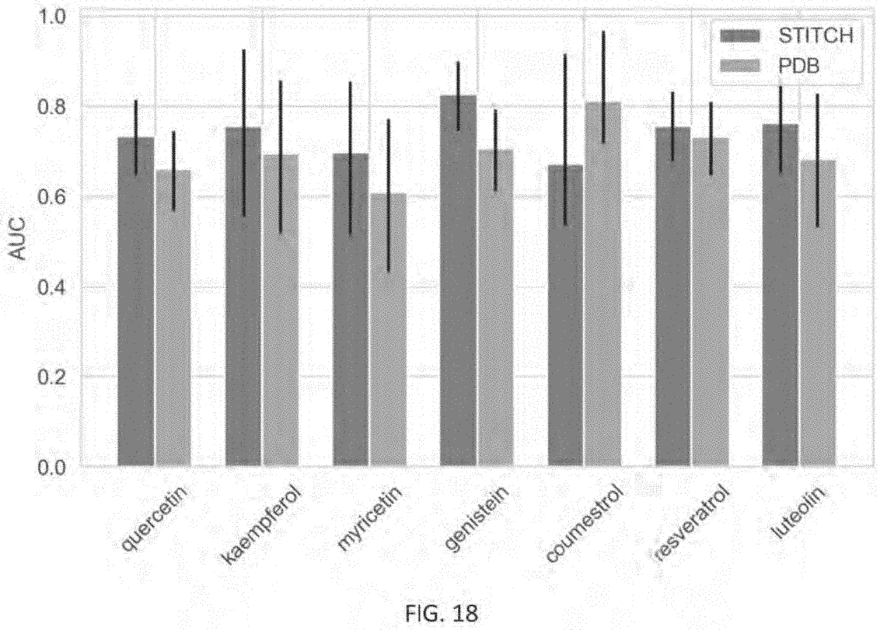

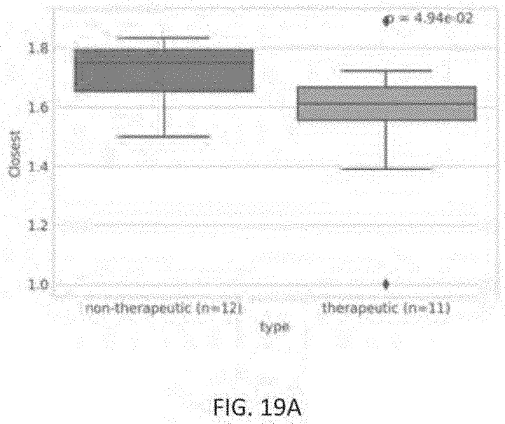

Finally, multiple robustness checks were performed to rule out the role of potential biases in the input data. To test if the predictions are biased by the set of known associations retrieved from CTD, 100 papers were randomly selected from PubMed containing MeSH terms that tag EGCG to diseases. The evidence was manually curated for EGCG's therapeutic effects for the diseases discussed in the published papers, excluding reviews and non-English language publications. The dataset was processed to include implicit associations, resulting in a total of 113 diseases associated with EGCG, of which 58 overlap with the associations reported by CTD (FIG. 7C). It was observed that the predictive power of the network proximity was unchanged whether the annotations from CTD, the manually curated list, or the union of both (FIG. 7D) were considered. To test the role of potential biases in the interactome, the analysis was repeated using a subset of the interactome derived from an unbiased high-throughput screening (FIG. 17) and only high-quality polyphenol-protein interactions retrieved from ligand-protein 3D resolved structures (FIG. 18). It was found that the predictive power was largely unchanged, indicating that the literature bias in the interactome does not affect the findings.

### Example 4: Network Proximity Predicts the Gene Expression Perturbation Induced by Polyphenols

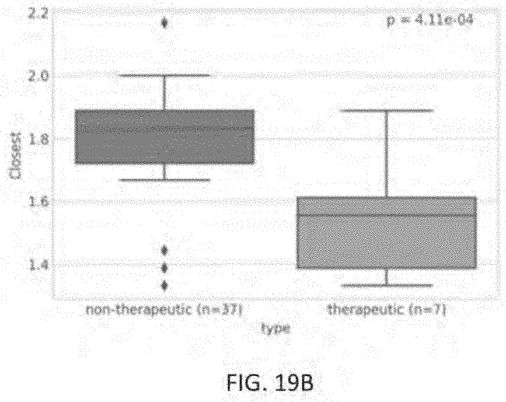

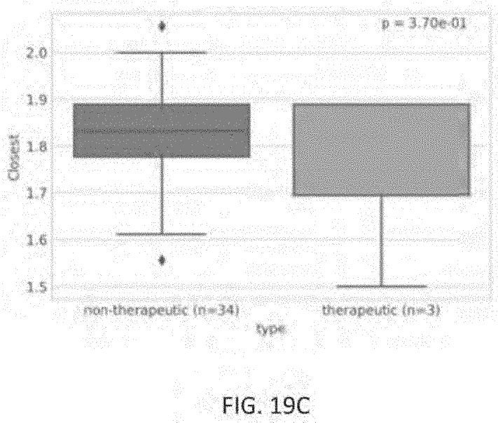

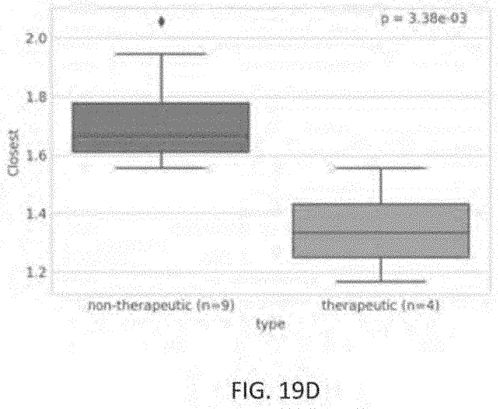

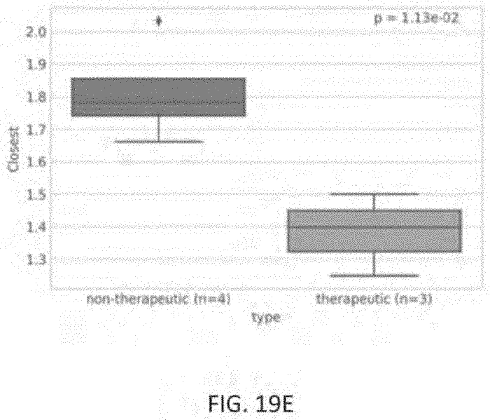

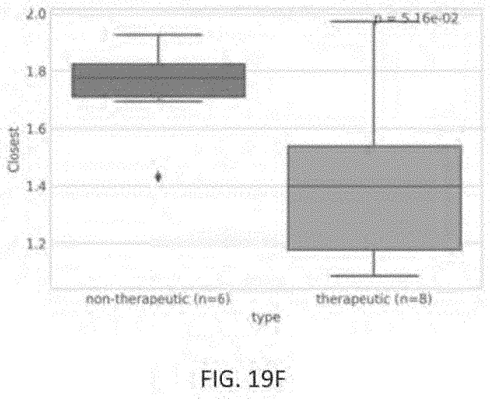

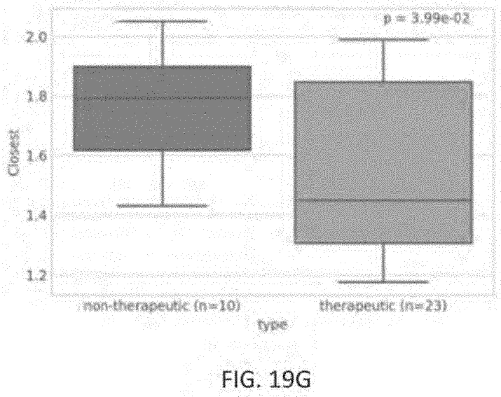

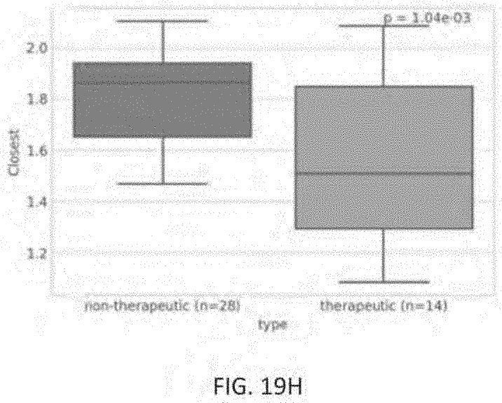

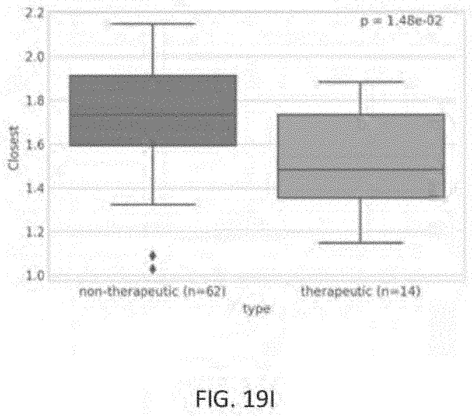

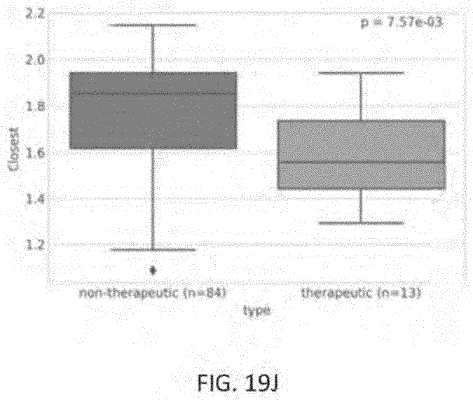

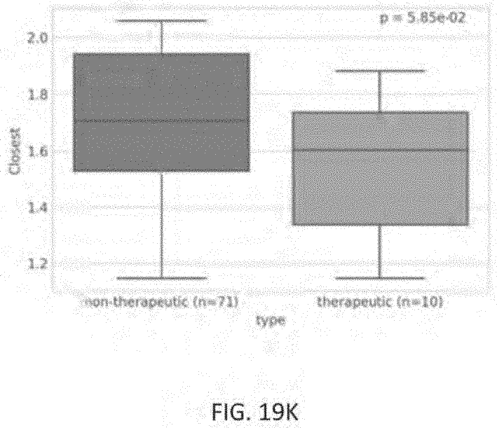

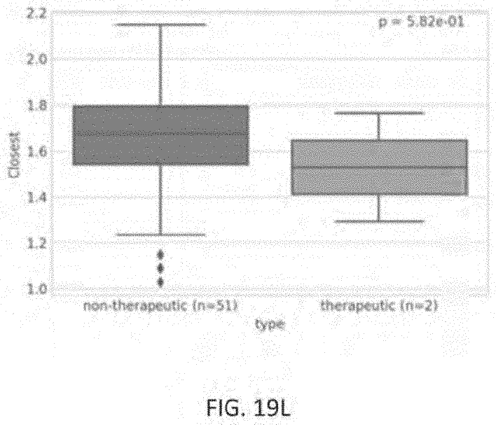

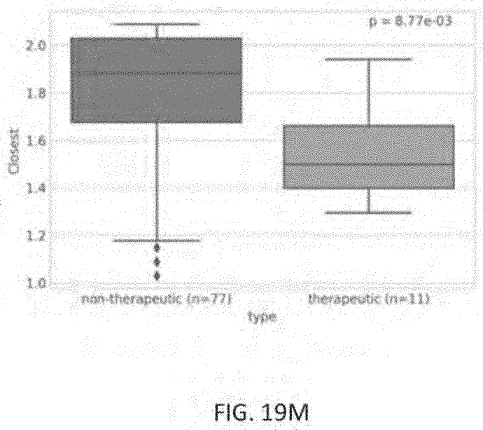

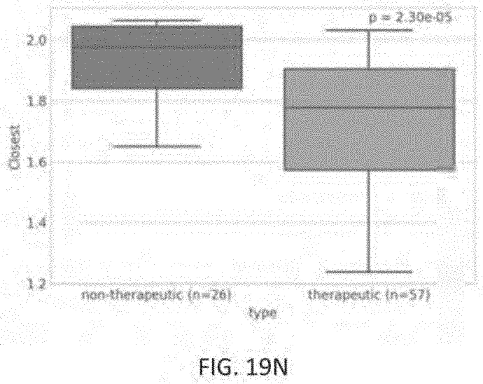

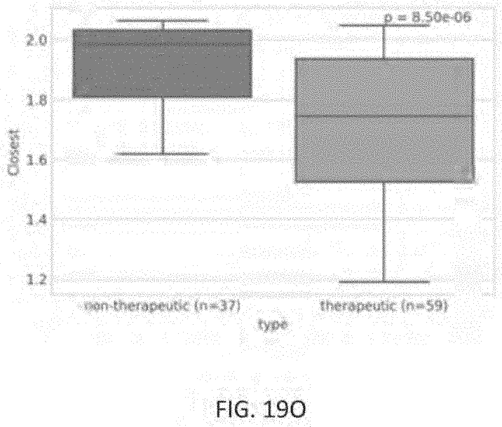

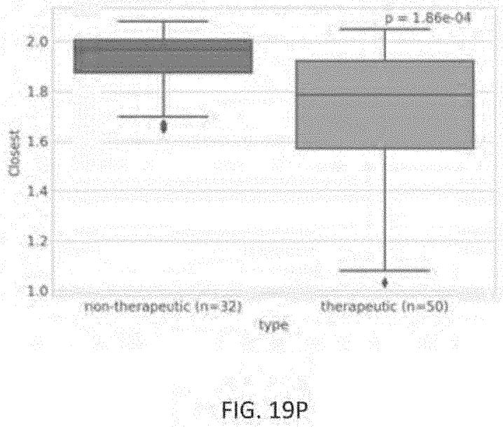

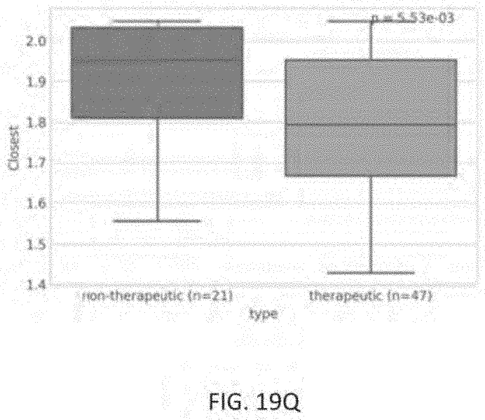

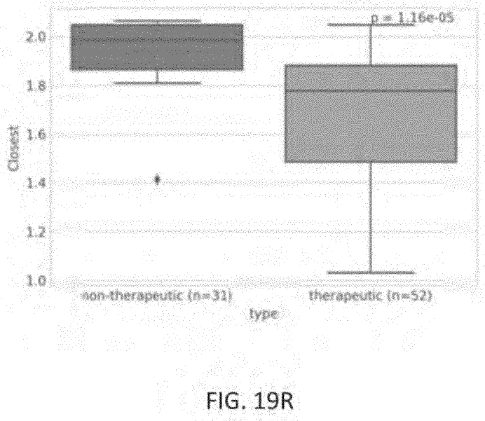

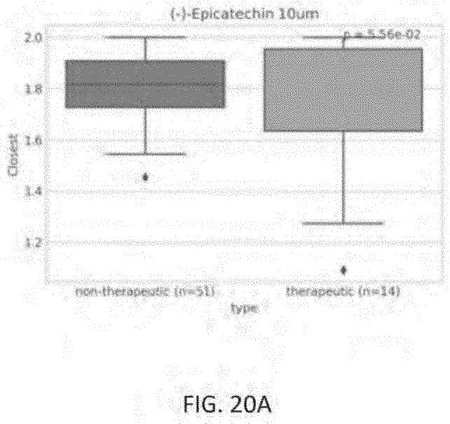

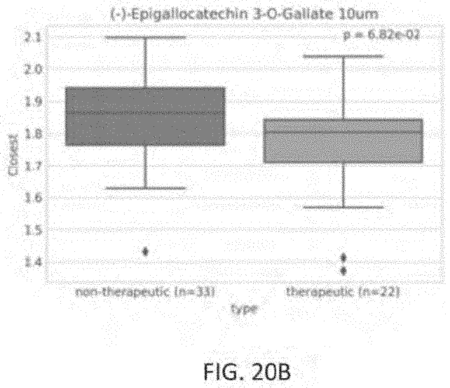

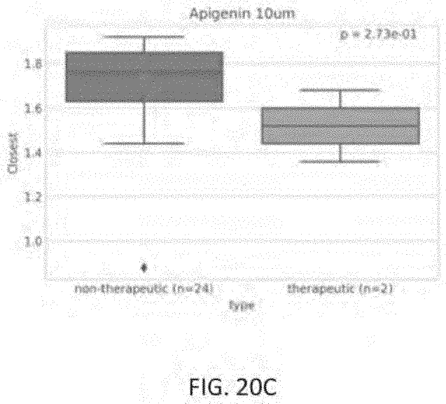

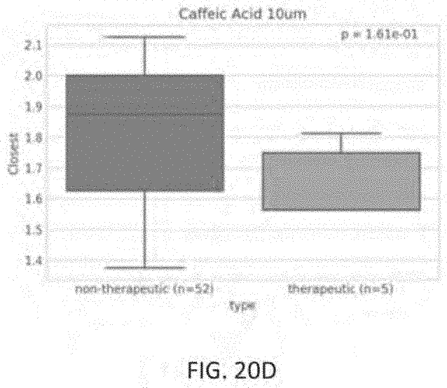

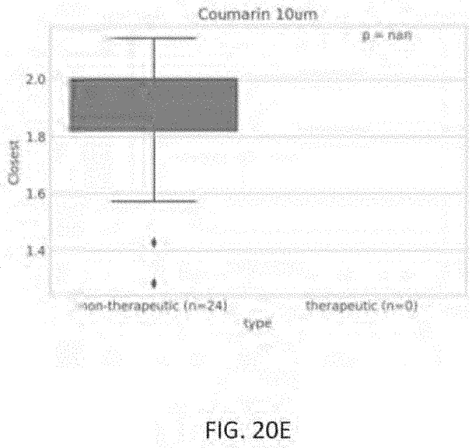

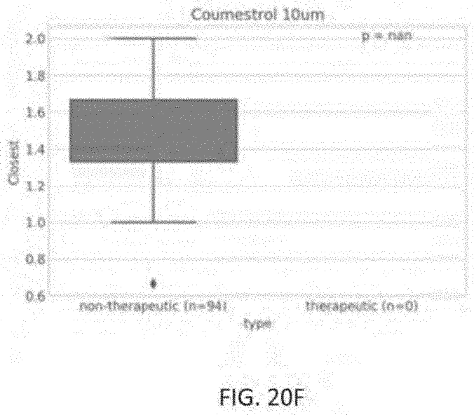

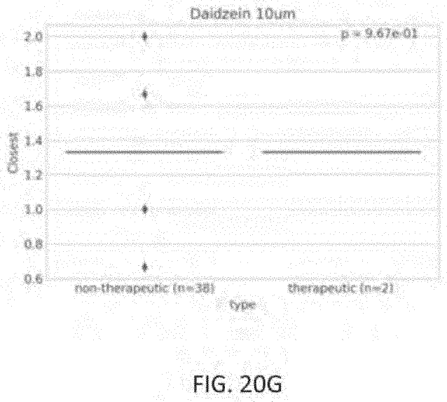

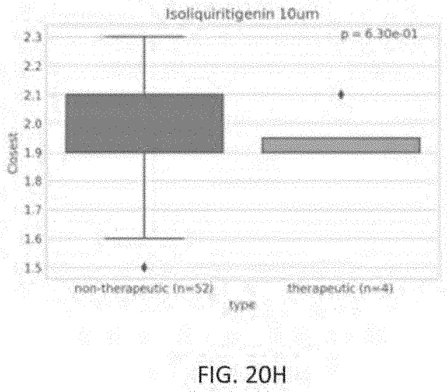

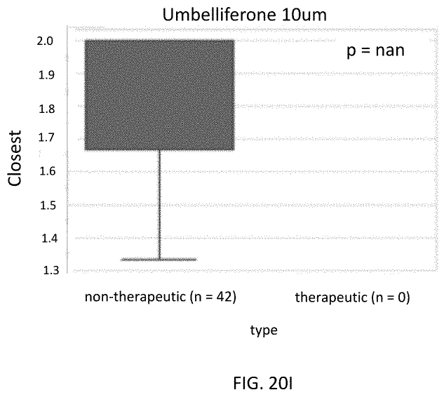

To validate the predicted polyphenol-disease associations expression perturbation signatures were retrieved from the Connectivity Map database for the treatment of the breast cancer MCF7 cell line with 22 polyphenols (Table 6). The database assigns each gene a z-score capturing the extent to which its expression is perturbed by a given polyphenol. The relationship between the extent in which polyphenols perturb the expression of disease genes, the network proximity between the polyphenol targets and disease proteins, and their known therapeutic effects was investigated (FIG. 8A). For example, different perturbation profiles for gene pools associated with different diseases were observed: for treatment with genistein (1 μM, 6 hours) 10 Skin Diseases (SD) genes with perturbation score>2 were observed, while only one highly perturbed Cerebrovascular Disorders (CD) was observed (FIG. 8B). Indeed, network proximity indicates that SD is closer to the genistein targets than CD, suggesting a relationship between network proximity, gene expression perturbation, and the therapeutic effects of the polyphenol (FIG. 8A). To test the validity of this hypothesis, an enrichment score was computed that measures the overrepresentation of disease genes among the most perturbed genes, finding 13 diseases that have their genes significantly enriched among the most deregulated genes by genistein, of which 4 have known therapeutic associations. It was found that these four diseases are significantly closer to the genistein targets than the nine diseases with non-therapeutic associations (FIGS. 8C-1-8C-4). A similar trend was observed for treatments with other polyphenols, whether the same (1 μM, FIGS. 8C-1-8C-4) or different (100 nM to 10 μM, FIG. 19 and FIG. 20) concentrations were used. This result suggests that changes in gene expression caused by a polyphenol is indicative of its therapeutic effects, but only if the observed expression change is limited to proteins proximal to the polyphenol targets (FIG. 8A).

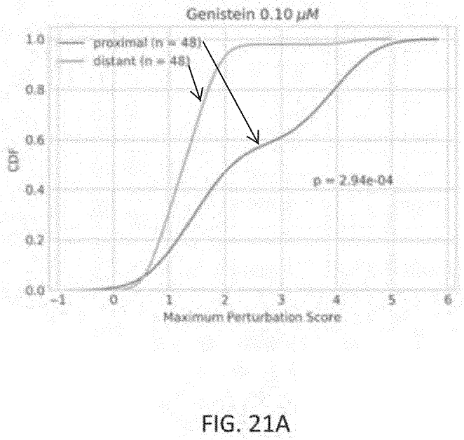

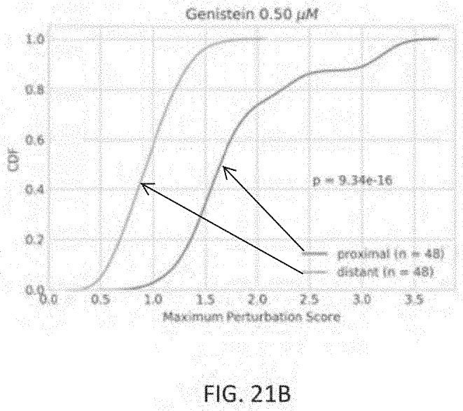

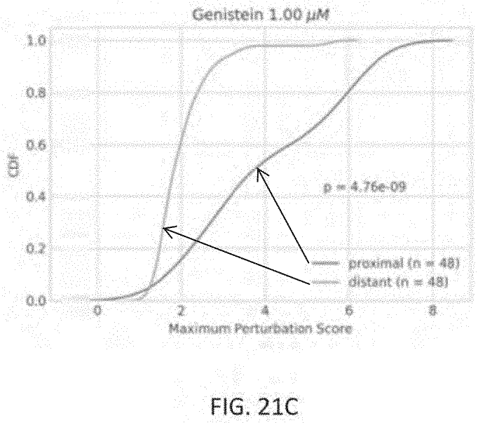

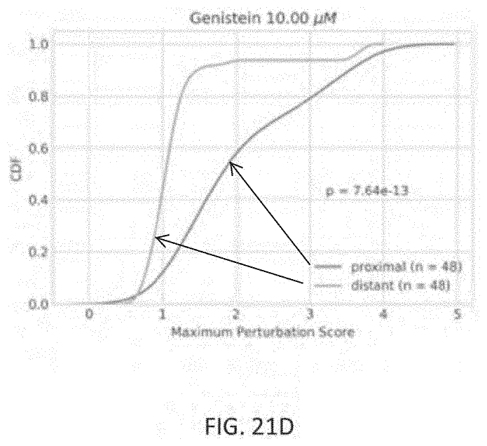

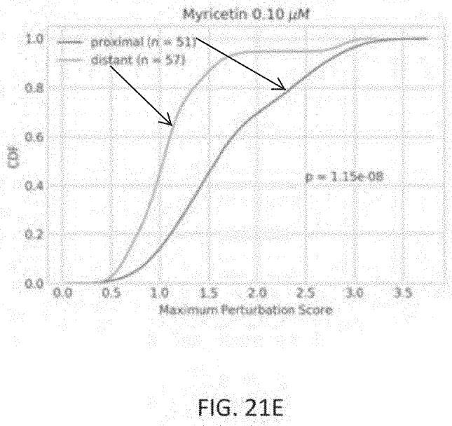

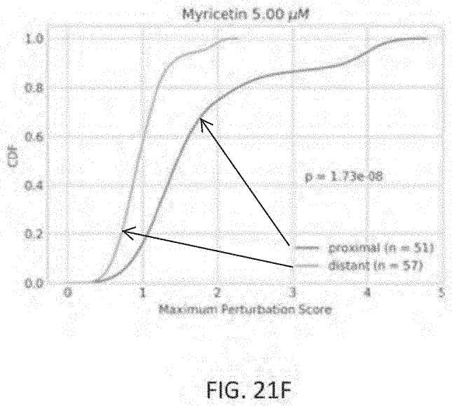

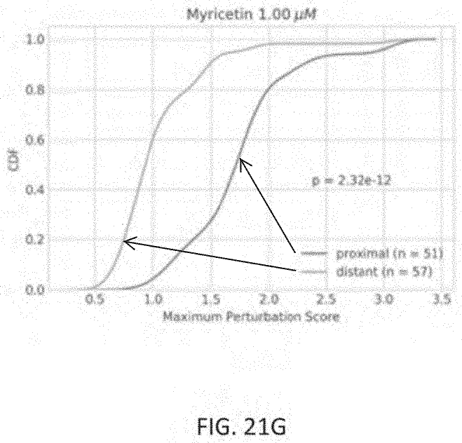

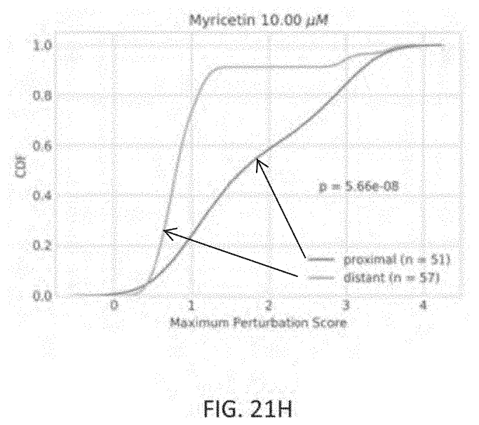

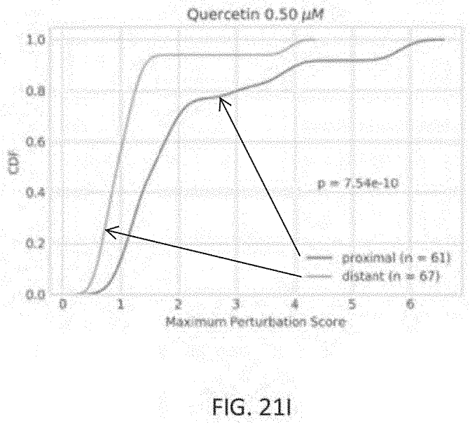

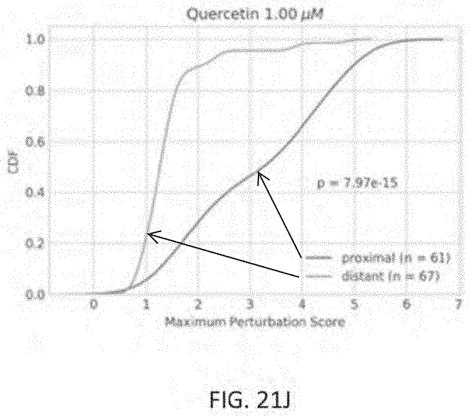

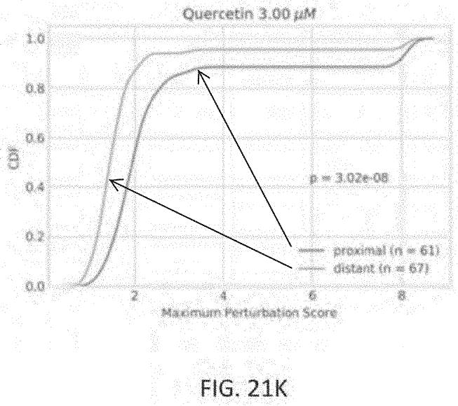

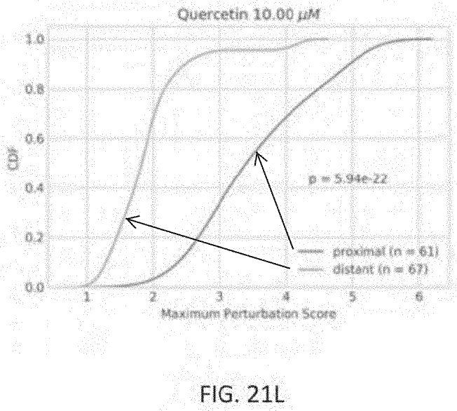

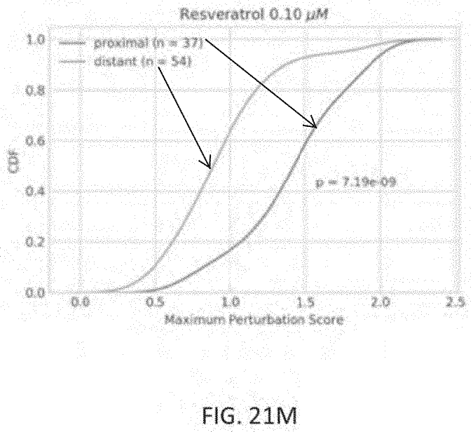

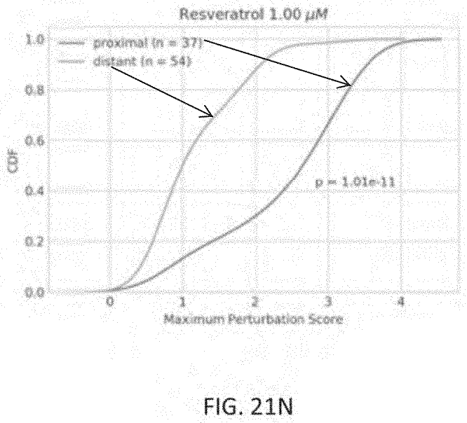

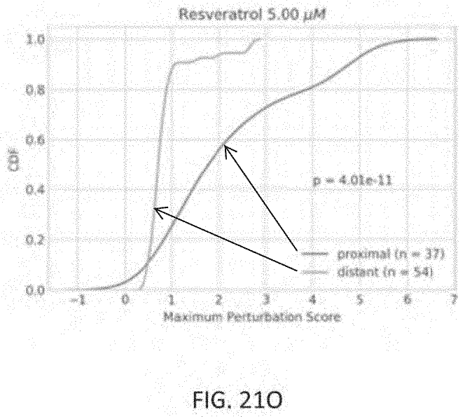

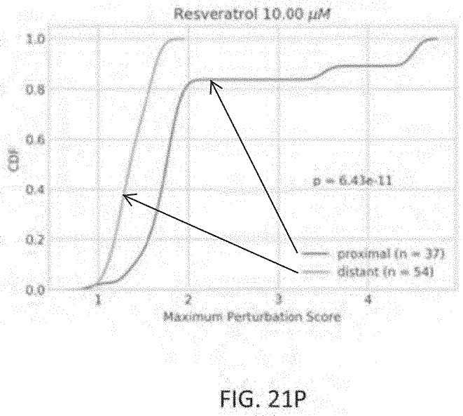

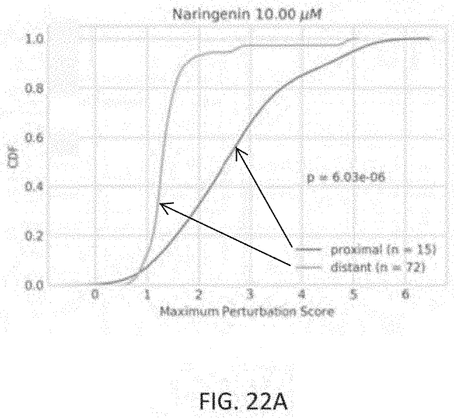

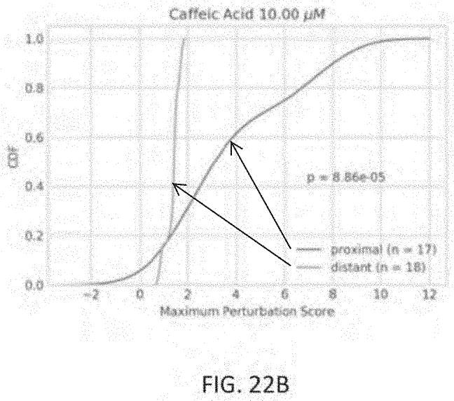

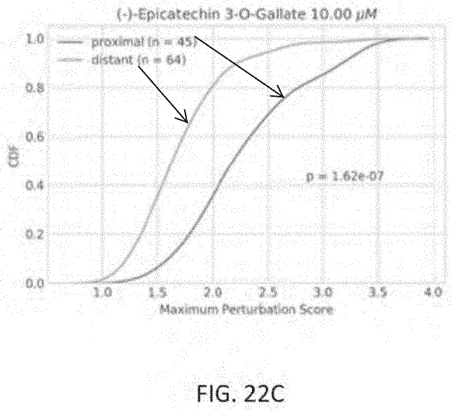

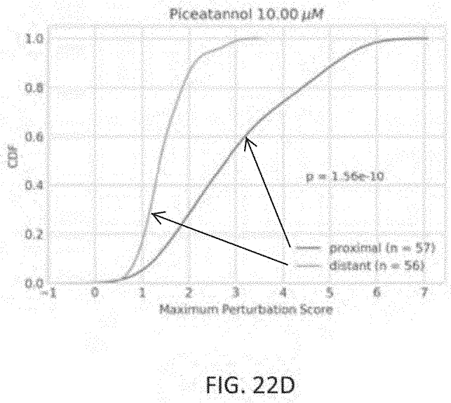

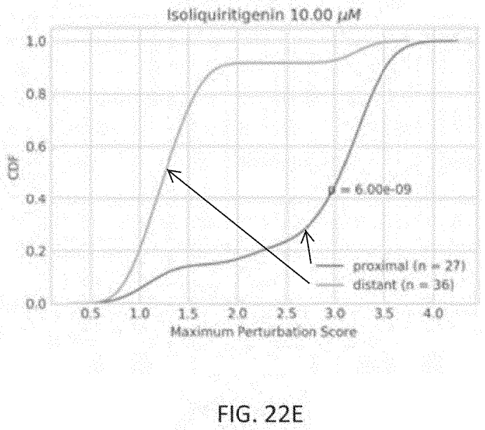

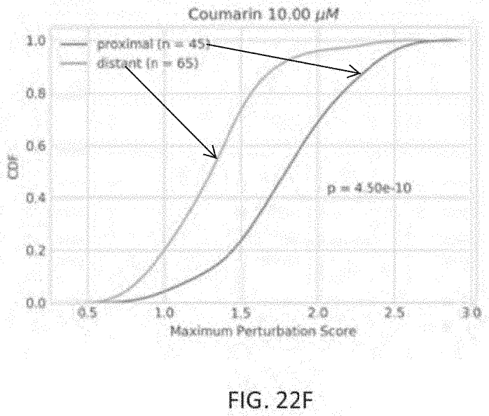

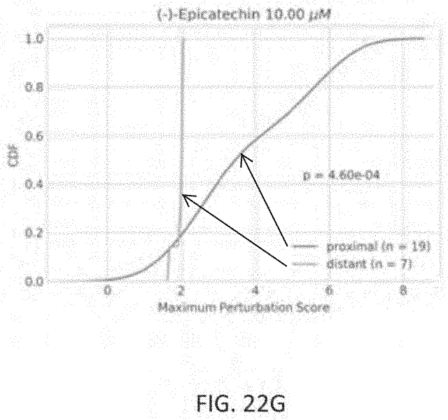

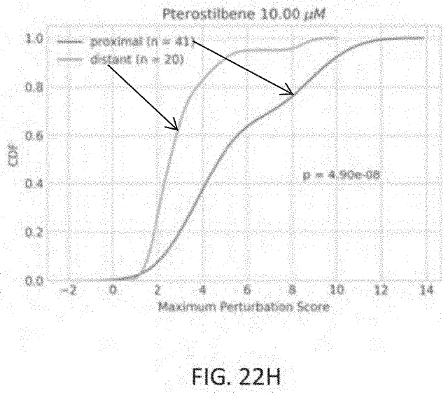

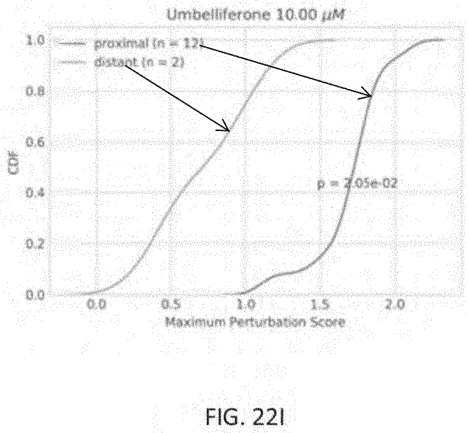

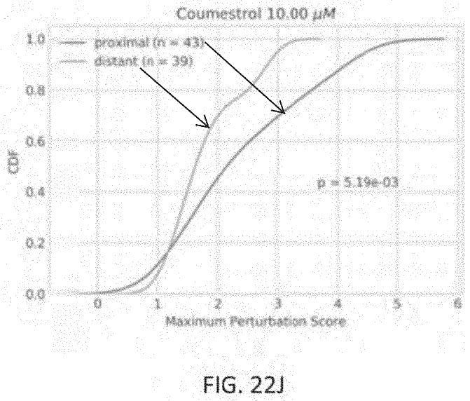

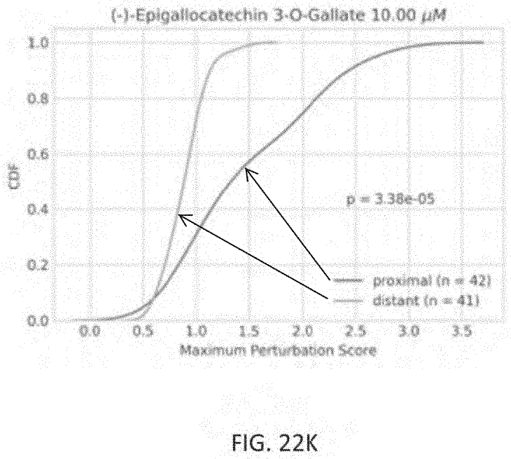

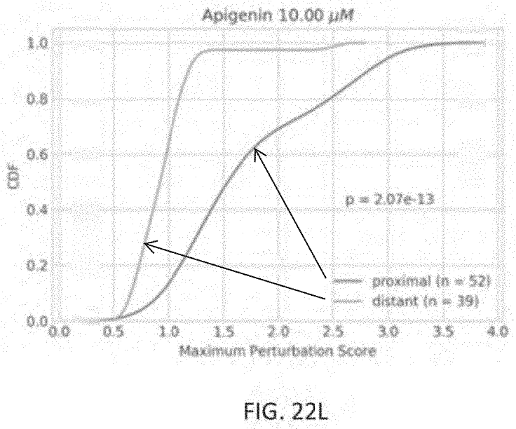

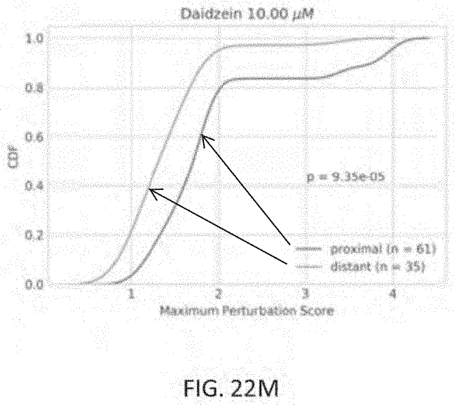

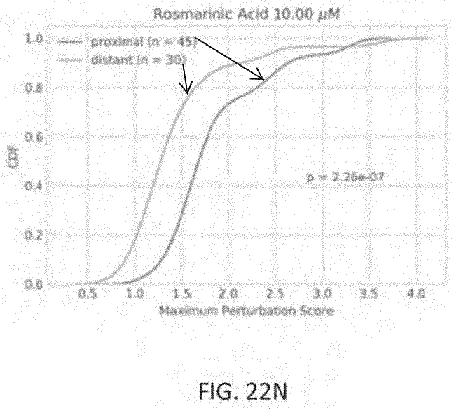

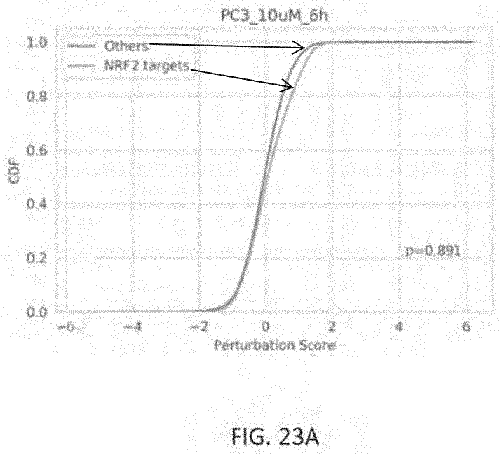

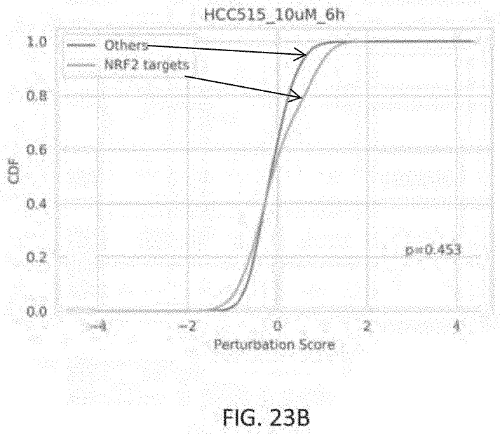

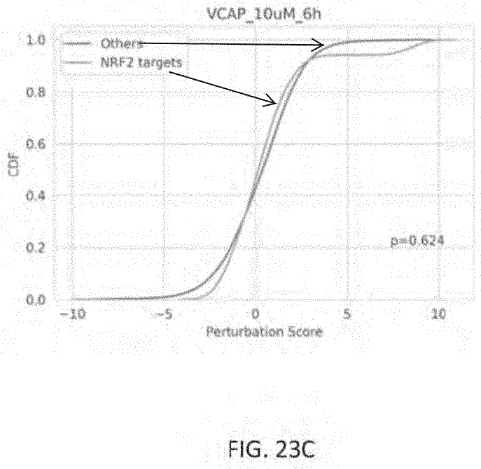

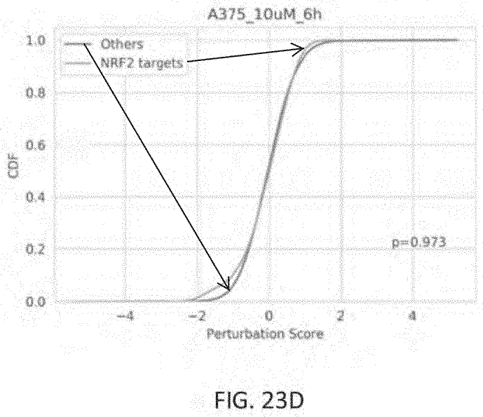

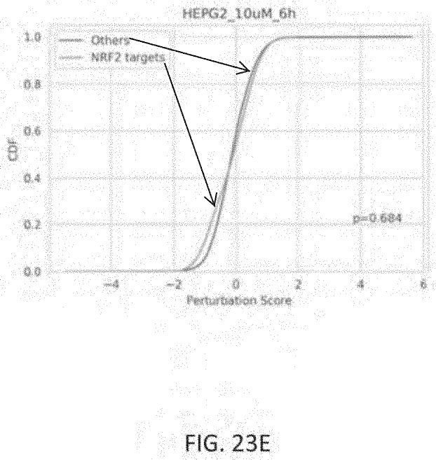

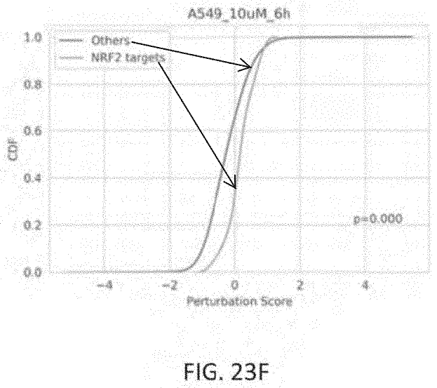

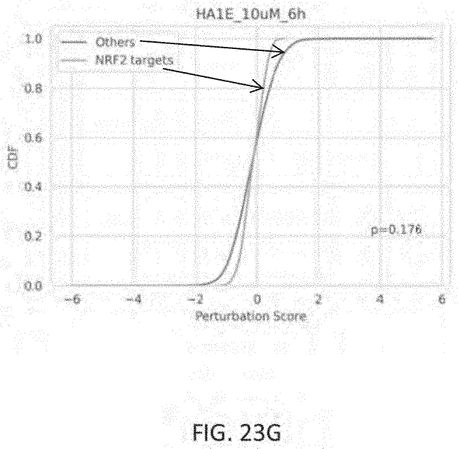

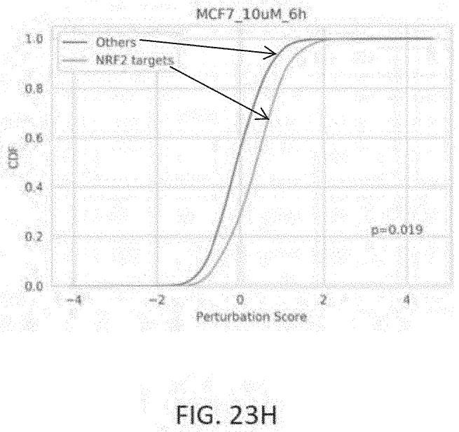

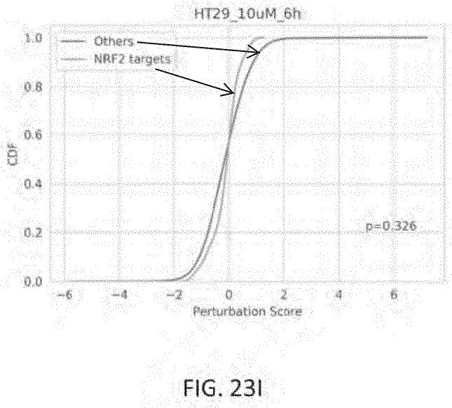

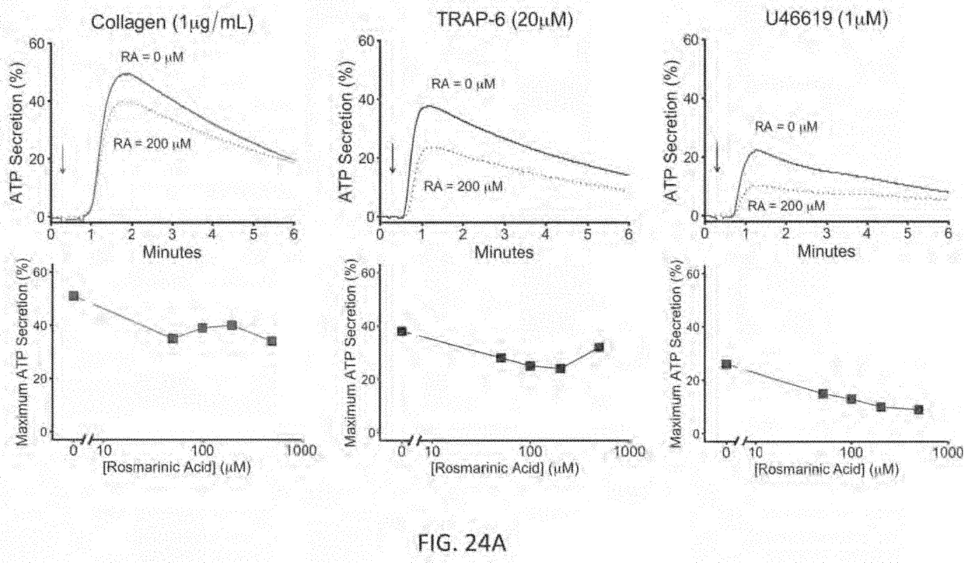

Network proximity can also be predictive of the overall gene expression perturbation caused by a polyphenol on the genes of a given disease. To test this, in each experimental combination defined by the polyphenol type and its concentration, the maximum perturbation score among genes for each disease was evaluated. The magnitude of the observed perturbation between diseases that were proximal (d_{c}<25th percentile, Z_{dc}<−0.5) or distal (d_{c}>75th percentile, Z_{dc}>−0.5) to the polyphenol targets were compared. FIGS. 9A and 9B show the results for the genistein treatment (1 μM, 6 hours), indicating that diseases proximal to the polyphenol targets show higher maximum perturbation values than distal diseases. The same trend was observed for other polyphenols (FIGS. 9B-1-9B-4, FIG. 21, and FIG. 22), confirming that the impact of a polyphenol on cellular signaling pathways is localized in the network space, being greater in the vicinity of the polyphenol targets compared to neighborhoods remote from these targets.

Taken together, these results indicate that network proximity offers a mechanistic interpretation for the gene expression perturbations induced by polyphenols, being also predictive of whether these perturbations result in therapeutic effects.

### Example 5: Unveiling the Mechanisms Responsible for the Therapeutic Effects of Specific Polyphenols

How the network-based framework can facilitate the mechanistic interpretation of the therapeutic effects of selected polyphenols was demonstrated, with a focus on Vascular Diseases (VD). Out of 65 polyphenols evaluated in this study, 27 were found to have associations to VD, as their targets were hitting the VD network neighborhood (Table 3). The targets of 15 out of the 27 polyphenols with 10 or less targets were inspected, as experimentally validating the mechanism of action among the interactions of more than 10 targets would provide complexities beyond the scope of this study. The network analysis identified direct links between biological processes related to vascular health and the targets of three polyphenols, gallic acid, rosmarinic acid, and 1,4-naphthoquinone (FIG. 10).

Gallic Acid: Gallic acid has a single human protein target, SERPINE1, which is also a VD-associated protein, resulting in d_c=0 and Z_dc=−3.02. SERPINE1 is involved in the regulation of blood clot dissolution and regulation of cell adhesion and spreading by modulating the proteins PLAT and PLAU, respectively. An inspection of the LCC formed by VD proteins also revealed that SERPINE1 directly interacts with the VD proteins PLG, LRP1, and F2 (FIG. 10), proteins directly or indirectly related to blood clot formation and dissolution, suggesting that these pathways may be involved in potential gallic acid mechanism of action. Indeed, recent studies using in vivo models report that gallic acid has protective effects on vascular health.

1,4-Naphthoquinone: 1,4-naphthoquinone targets four proteins, MAP2K1, MAOA, CDC25B and IDO1, which are proximal to VD-associated proteins (d_c=1.25, [symbol] Z [symbol] _dc=−1.51) (FIG. 10). Indeed, the derivative compounds of 1,4-naphthoquinone have been explored as therapeutic agents for centuries. The polyphenol might influence biological processes related to vascular diseases through the action of its target MAP2K1, a gene involved in signaling pathways related to vascular smooth cell contraction and VEGF signaling, which also interacts with 5 VD associated proteins (FIG. 10). Mutations in MAP2K1 gene have been proposed as a cause of extracranial arteriovenous malformation as a result of endothelial cell dysfunction due to increased MEK1 activity. Additionally, one of 1,4-naphthoquinone derivatives, shikonin, was shown to modulate inflammatory responses, protecting against brain ischemic damage.

Rosmarinic Acid: Rosmarinic acid (RA) can bind to three human proteins, FYN, MCL1, and AKR1B1, offering a statistically significant proximity to VD genes (d_c=1.00, [symbol] Z [symbol] _dc=−1.38). The analysis of the RA target FYN and three of its seven direct neighbors in the VD module (CD36, APP, and PRKCH) suggests the role of this polyphenol on platelet function—cells specialized in blood clot formation and involved in abnormal clotting that can lead to heart attacks and stroke. FYN also directly interacts with NFE2L2 (also known as NRF2), a transcription factor that regulates the expression of several genes with anti-oxidant properties43. Using RA perturbation profiles from the Connectivity Map database, it was observed that two cell lines (A549, MCF7) showed higher perturbation scores for genes that are directly regulated by NFE2L2 after treatment with RA. Indeed, recent reports show that mice lacking FYN have reduced platelet activit and that RA's protective effects on vascular calcification and on aortic endothelial function after diabetes-induced damage is mediated by anti-oxidant mechanisms. These observations suggest that RA activity might be mediated by FYN, ultimately regulating the processes of platelet activity and expression of anti-oxidant genes. The RA target MCL1 has also been proposed as an essential survival factor for endothelial cells in blood vessel production during angiogenesis, and it has been observed that RA has been found to restore cardiac function in rat models of ischemia/reperfusion injury.

In summary, by integrating literature evidence and by inspecting the polyphenol targets and their neighbors in the interactome, the molecular mechanisms underlying the protective effects of gallic acid, rosmarinic acid, and 1,4-naphthoquinone for VD were identified. The analysis suggests that gallic acid activity involves blood clot dissolution processes, rosmarinic acid acts on platelet activation and anti-oxidant pathways through FYN and its neighbors, and 1,4-naphthoquinone acts on signaling pathways of vascular cells through MAP2K1 activity.

### Example 6: Experimental Evidence Confirms that Rosmarinic Acid Modulates Platelet Function

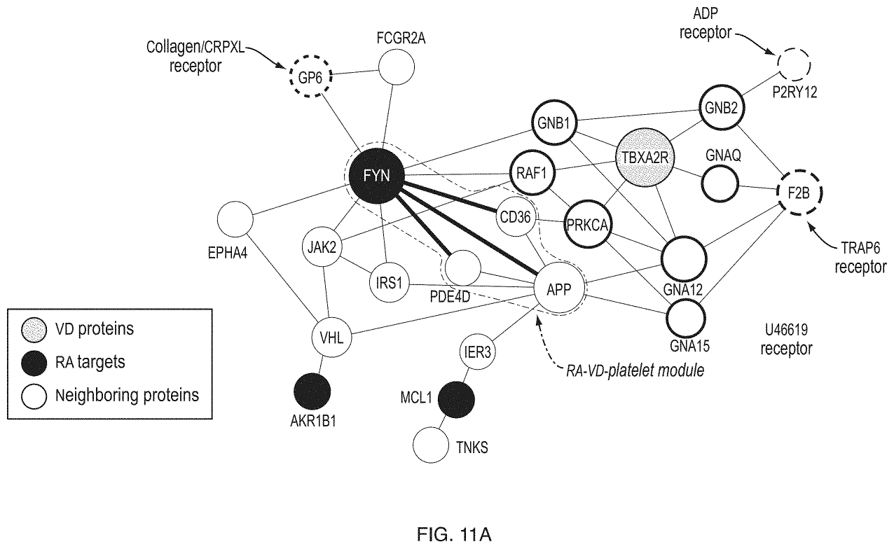

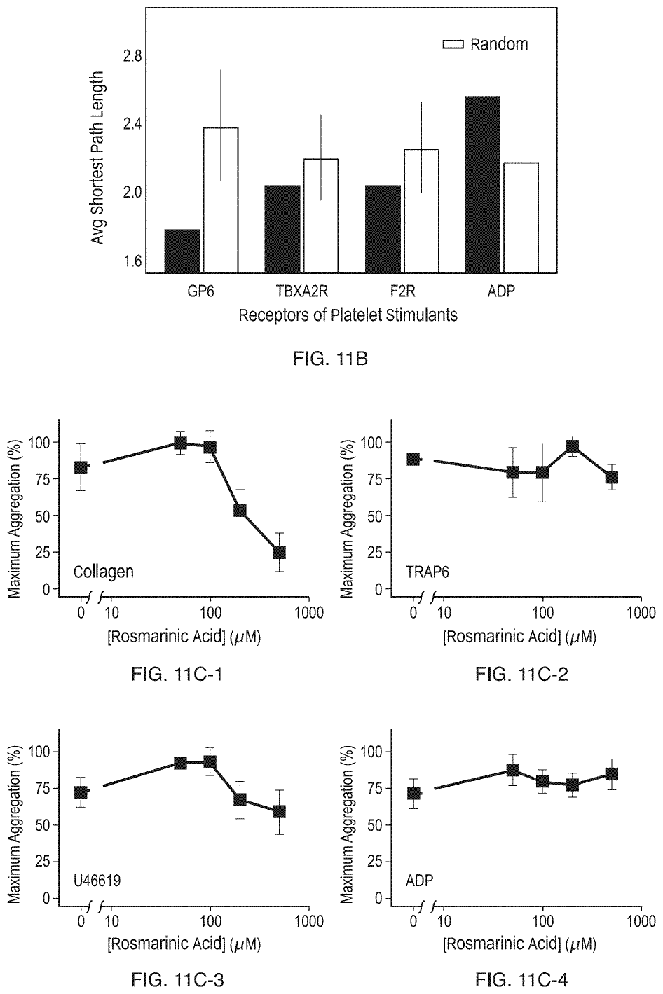

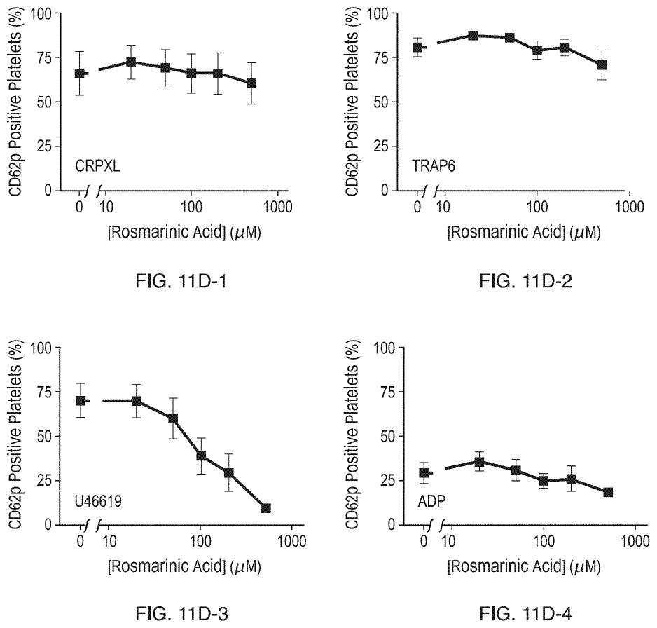

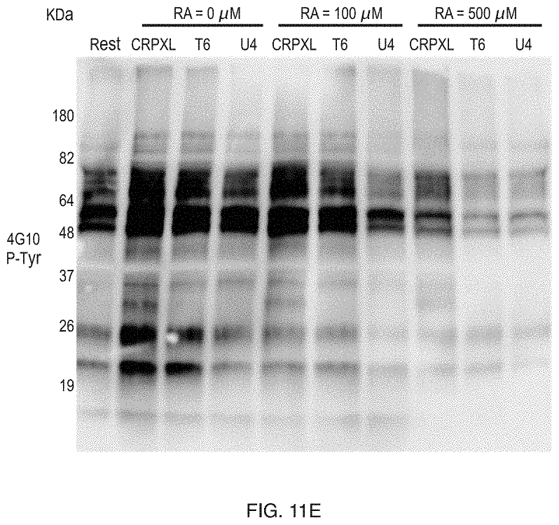

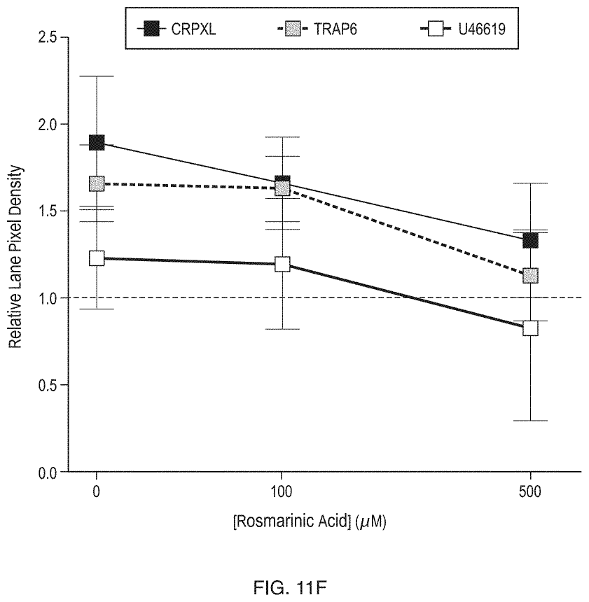

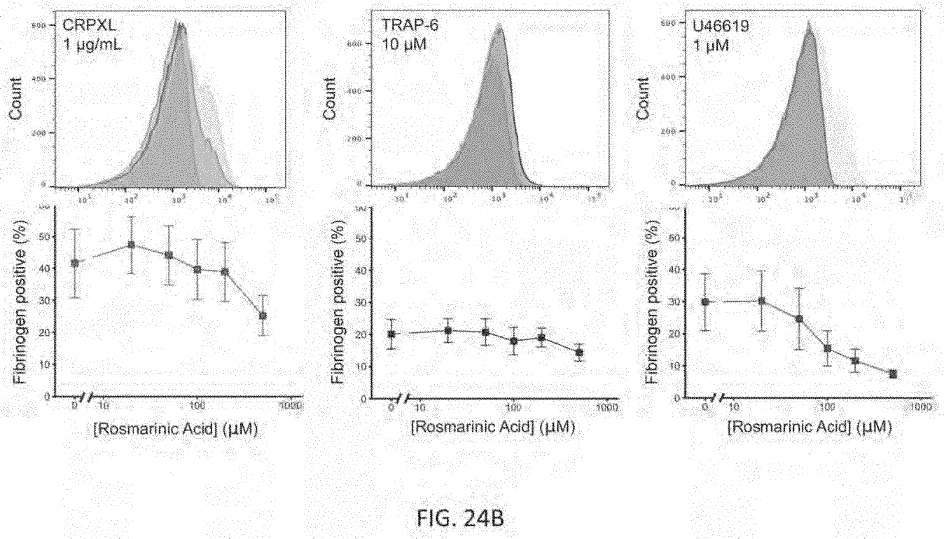

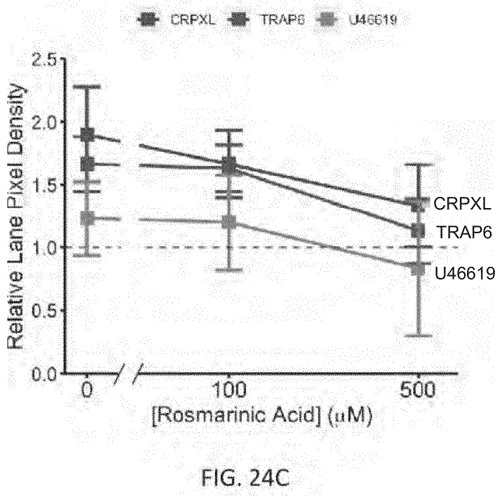

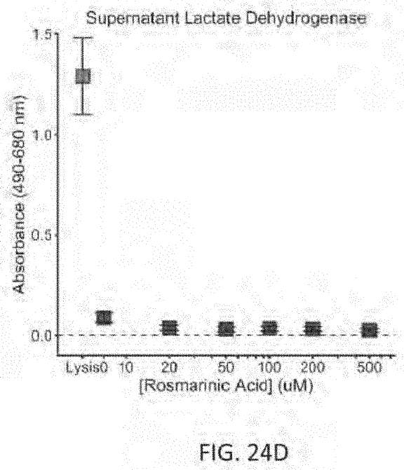

To validate the predictive power of the developed framework, direct experimental evidence of the predicted mechanistic role of Rosmarinic acid (RA) in VD was sought. The VD network neighborhood shows that RA targets are in close proximity to proteins related to platelet function, cells that control blood clot formation and whose inhibition is the mechanism underlying drugs prescribed to prevent heart attack and stroke. FIG. 11A shows the interactome region containing identified the RA-VD-platelet module: the connected component formed by the RA target FYN and the VD proteins associated to platelet function PDE4D, CD36, and APP; as well as its distance to the receptors of known platelet activators (FIG. 11A). Therefore, whether RA influenced platelet activation in vitro was evaluated. As platelets can be stimulated through different activation pathways, RA effects can, in principle, occur in any of them. To test these different possibilities, platelets were pretreated with RA and then activated with: 1) glycoprotein VI by collagen or collagen-related peptide (Collagen/CRPXL); 2) protease-activated receptors-1,4 by thrombin receptor activator peptide-6 (TRAP-6); 3) prostanoid thromboxane receptor by the thromboxane A2 analogue (U46619); and 4) P2Y1/12 receptor stimulation by adenosine diphosphate (ADP). When the network distance between each stimulant receptor and the RA-VD-platelet module (FIG. 11A) was compared, it was observed that the receptors for Collagen/CRPXL, TRAP-6, and U46619 are closer than the random expectation, while the receptor for ADP is more distant (FIG. 11B). It is expected that platelets would be most affected by RA when treated with stimulants whose receptors are most proximal to the RA-VD-platelet module, i.e. Collagen/CRPXL, TRAP-6, and U46619. As a control, no effect is expected for the distant receptor ADP. The experiments confirmed this prediction: RA inhibits collagen-mediated platelet aggregation (FIGS. 11C-1-11C-4) and impairs dense granule secretion induced by CRPXL, TRAP-6 and U46619 (FIG. 24). RA-treated platelets also displayed dampened alpha granule secretion (FIGS. 11D-1-11D-4) and integrin αIIbβ3 activation (FIG. 24) in response to U46619. As expected, RA did not affect platelet functions when a stimulant whose receptor is distant from the RA-VD-module was used. These findings suggest strong network effects is the way RA impairs several basic hallmarks of platelet activation, supporting that the proximity between RA targets and the functional neighborhood associated to platelet function (FIG. 11A) can explain RA impact on VD.

The molecular mechanisms involved in the functional impact of RA on platelets was clarified. The RA target FYN is a protein-tyrosine kinase and platelet activation is coordinated by several kinases that phosphorylate adaptors, enzymes, and cytoskeletal proteins downstream of platelet surface receptors. Given this connection, RA may inhibit platelets function by blocking agonist-induced protein tyrosine phosphorylation. It was observed that RA-treated platelets demonstrated a dose-dependent reduction in total tyrosine phosphorylation in response to CRPXL, TRAP-6 and U46619 (FIGS. 11E, 11F). This indicates that RA perturbs the phospho-signaling networks that regulate platelet response to extracellular stimuli.

Altogether, these findings support the prediction that RA, by targeting a network neighborhood related to platelet function, modulates platelet activation and function. It also supports the observation that its mechanism of action involves the protein-tyrosine kinase FYN (FIG. 11A) and the inhibition of tyrosine phosphorylation. Finally, while polyphenols are usually known for the health benefits caused by their antioxidant function, here another mechanism pathway through which they could benefit health is illustrated, in particular, by affecting platelet function.

### Example 7: Methods: Building the Interactome

The human interactome was assembled from 16 databases containing different types of protein-protein interactions (PPIs): 1) binary PPIs tested by high-throughput yeast-two-hybrid (Y2H) experiments; 2) kinase-substrate interactions from literature-derived low-throughput and high-throughput experiments from KinomeNetworkX, Human Protein Resource Database (HPRD), and PhosphositePlus; 3) carefully literature-curated PPIs identified by affinity purification followed by mass spectrometry (AP-MS), and from literature-derived low-throughput experiments from InWeb, BioGRID, PINA, HPRD, MINT, IntAct, and InnateDB; 4) high-quality PPIs from three-dimensional (3D) protein structures reported in Instruct, Interactome3D, and INSIDER; 5) signaling networks from literature-derived low-throughput experiments as annotated in SignaLink2.0; and 6) protein complex from BioPlex2.0. The genes were mapped to their Entrez ID based on the National Center for Biotechnology Information (NCBI) database as well as their official gene symbols. The resulting interactome includes 351,444 protein-protein interactions (PPIs) connecting 17,706 unique proteins. The largest connected component has 351,393 PPIs and 17,651 proteins.

### Example 8: Methods: Polyphenols, Polyphenol targets, and Disease Proteins

The 759 polyphenols were retrieved from the PhenolExplorer database. The database lists polyphenols with food composition data or profiled in biofluids after interventions with polyphenol-rich diets. For the analysis, only polyphenols that: 1) could be mapped in PubChem IDs, 2) were listed in the Comparative Toxicogenomics (CTD) database as having therapeutic effects on human diseases, and 3) had protein-binding information present in the STITCH database with experimental evidence were considered (FIG. 5A). After these steps, a final list of 65 polyphenols was considered, for which 598 protein targets were retrieved from STITCH (Table 4). The 3,173 disease proteins considered corresponded to 299 diseases retrieved from Menche, J. et al. “Disease networks. Uncovering disease-disease relationships through the incomplete interactome.” Science 347, 1257601 (2015). Gene ontology enrichment analysis on protein targets was performed using the Bioconductor package clusterProfiler with a significance threshold of p<0.05 and Benjamini-Hochberg multiple testing correction with q<0.05.

### Example 9: Methods: Polyphenol Disease Associations

The polyphenol-disease associations were retrieved from the Comparative Toxicogenomics Database (CTD). Only manually curated associations labeled as therapeutic were considered. By considering the hierarchical structure of diseases along the MeSH tree, the study expanded explicit polyphenol-disease associations to include also implicit associations. This procedure was performed by propagating associations in the lower branches of the MeSH tree to consider also the diseases in the higher levels of the same tree branch. For example, a polyphenol associated with ‘heart diseases’ would also be associated to the more general category of ‘cardiovascular diseases’. By performing this expansion, a final list of 1,525 known associations between the 65 polyphenols and the 299 diseases considered in this study was obtained.

### Example 10: Methods: Network Proximity Between Polyphenol Targets and Disease Proteins

The proximity between a disease and a polyphenol was evaluated using a distance metric that takes into account the shortest path lengths between polyphenol targets and disease proteins. Given S, the set of disease proteins, T, the set of polyphenol targets, and d(s,t), the shortest path length between nodes s and t in the network, it is defined:

$${d}_{c} ( S , T ) = \frac{1}{‖ T ‖} {Σ}_{t ∈ T} {min}_{s ∈ S} d ( s , t ) \qquad [ 1 ]$$

To assess the significance of the distance between a disease and a polyphenol (S, T), a reference distance distribution was created corresponding to the expected distances between two randomly selected groups of proteins matching the size and degrees of the original disease proteins and polyphenol targets in the network. The reference distance distribution was generated by calculating the proximity between these two randomly selected groups, a procedure repeated 1,000 times. The mean μ_(d(S,T)) and s.d. σ_(d(S,T)) of the reference distribution were used to convert the absolute distance d_c to a relative distance Z_dc, defined as:

$${Z}_{dc} = \frac{d - {μ}_{{d}_{c} ( S , T )}}{{σ}_{{d}_{c} ( S , T )}} \qquad [ 2 ]$$

Due to the scale-free nature of the human interactome, there are few nodes with high degrees and to avoid repeatedly choosing the same (high degree) nodes, a degree-preserving random selection was performed.

### Example 11: Methods: Area Under ROC Curve Analysis

For each polyphenol, AUC was used to evaluate how well the network proximity distinguishes diseases with known therapeutic associations from all the others of the set of 299 diseases. The set of known associations (therapeutic) retrieved from CTD were used as positive instances, all unknown associations were defined as negative instances, and the area under the ROC curve was computed using the implementation in the Scikit-learn Python package. Furthermore, 95% confidence intervals were calculated using the bootstrap technique with 2,000 resamplings with sample sizes of 150 each. Considering that AUC provides an overall performance, a metric to evaluate the top-ranking predictions was used. For this analysis, the precision of the top 10 predictions was calculated, considering only the polyphenol-disease associations with relative distance Z_dc<−0.520.

### Example 12: Methods: Analysis of Network Proximity and Gene Expression Deregulation

Perturbation signatures were retrieved from the Connectivity Map database for the MCF7 cell line after treatment with 22 polyphenols. These signatures reflect the perturbation of the gene expression profile caused by the treatment with that particular polyphenol relative to a reference population, which comprises all other treatments in the same experimental plate. For polyphenols having more than one experimental instance (time of exposure, cell line, dose), the one with highest distil_cc_q75 value (75th quantile of pairwise spearman correlations in landmark genes) was selected. Gene Set Enrichment Analysis was performed to evaluate the enrichment of disease genes among the top deregulated genes in the perturbation profiles. This analysis offers an Enrichment Scores (ES) that have small values when genes are randomly distributed among the ordered list of expression values and high values when they are concentrated at the top or bottom of the list. The ES significance was calculated by creating 1,000 random selection of gene sets with the same size as the original set and calculating an empirical p-value by considering the proportion of random sets resulting in ES smaller than the original case. The p-values were adjusted for multiple testing using the Benjamini-Hochberg method. The network proximity d_c of disease proteins and polyphenol targets for diseases with significant ES were compared according to their therapeutic and non-therapeutic associations using the Student's t-test.

### Example 13: Methods: Platelet Isolation

Human blood collection was performed as previously described in accordance with the Declaration of Helsinki and ethics regulations with Institutional Review Board approval from Brigham and Women's Hospital (P001526). Healthy volunteers did not ingest known platelet inhibitors for at least 10 days prior. Citrated whole blood underwent centrifugation with a slow break (177×g, 20 minutes) and the PRP fraction was acquired for subsequent experiments. For washed platelets, PRP was incubated with 1 μM prostaglandin E1 (Sigma, P5515) and immediately underwent centrifugation with a slow break (1000×g, 5 minutes). Platelet-poor plasma was aspirated, and pellets resuspended in platelet resuspension buffer (PRB; 10 mM Hepes, 140 mM NaCl, 3 mM KCl, 0.5 mM MgCl2, 5 mM NaHCO3, 10 mM glucose, pH 7.4).

### Example 14: Methods: Platelet Aggregometry

Platelet aggregation was measured by turbidimetric aggregometry. Briefly, PRP was pretreated with RA for 1 hour before adding 250 μL to siliconized glass cuvettes containing magnetic stir bars. Samples were placed in Chrono-Log® Model 700 Aggregometers before the addition of various platelet agonists. Platelet aggregation was monitored for 6 minutes at 37° C. with a stir speed of 1000 rpm and the maximum extend of aggregation recorded using AGGRO/LINK®8 software. In some cases, dense granule release was simultaneously recorded by supplementing samples with Chrono-Lume® (Chrono-Log®, 395) according to the manufacturer's instructions.

### Example 15: Methods: Platelet Alpha Granule Secretion and Integrin αIIbβ3 Activation

Changes in platelet surface expression of P-selectin (CD62P) or binding of Alexa Fluor™ 488-conjugated fibrinogen were used to assess alpha granule secretion and integrin αIIbβ3 activation, respectively. First, PRP was pre-incubated with RA for 1 hour, followed by stimulation with various platelet agonists under static conditions at 37° C. for 20 minutes. Samples were then incubated with APC-conjugated anti-human CD62P antibodies (BioLegend®, 304910) and 100 μg/mL Alexa Fluor™ 488-Fibrinogen (Thermo Scientific™, F13191) for 20 minutes, before fixation in 2% [v/v] paraformaldehyde (Thermo Scientific™, AAJ19945K2). 50,000 platelets were processed per sample using a Cytek™ Aurora spectral flow cytometer. Percent-positive cells were determined by gating on fluorescence intensity compared to unstimulated samples.

### Example 16: Methods: Platelet Cytotoxicity

Cytotoxicity were tested by measuring lactate dehydrogenase (LDH) release by permeabilized platelets into the supernatant. Briefly, washed platelets were treated with various concentrations of RA for 1 hour, before isolating supernatants via centrifugation (15,000×g, 5 min). A Pierce LDH Activity Kit (Thermo Scientific™, 88953) was then used to assess supernatant levels of LDH.

### Example 17: Methods: Platelet Phosphorylation

Washed platelets were pre-treated with RA for 1 hour, followed by agonist stimulation for 10 minutes. Platelets were lysed on ice with RIPA Lysis Buffer System® (Santa Cruz®, sc-24948) and sample supernatants clarified via centrifugation (14,000 rpm, 5 min, 4° C.). Supernatants were reduced with Laemmli Sample Buffer (Bio-Rad, 1610737) and proteins separated by molecular weight in PROTEAN TGX™ precast gels (Bio-Rad, 4561084). Proteins were transferred to PVDF membranes (Bio-Rad, 1620174) and probed with 4G10 (Milipore, 05-321), a primary antibody clone that recognizes phosphorylated tyrosine residues. Membranes were probed with horseradish peroxidase-conjugated secondary antibodies (Cell Signaling Technologies, 7074S) to catalyze an electrochemiluminescent reaction (Thermo Scientific™, PI32109). Membranes were visualized using a Bio-Rad ChemiDoc Imaging System and densitometric analysis of protein lanes conducted using ImageJ (NIH, Version 1.52a).

### Example 18: Discussion

Here, a network-based framework was proposed to predict, in an experimentally falsifiable fashion, the therapeutic effects of dietary polyphenols in human diseases. It was found that polyphenol protein targets cluster in specific functional neighborhoods of the interactome, and shown that the network proximity between polyphenol targets and disease proteins is predictive of the therapeutic effects of polyphenols on specific diseases. Predictions were validated by demonstrating that diseases whose proteins are proximal to polyphenol targets tend to have significant changes in gene expression in cell lines treated with the respective polyphenol, while such changes are absent for diseases whose proteins are distal to polyphenol targets. Finally, as a novel prediction, it was found that the network neighborhood in which rosmarinic acid (RA) targets are proximal to vascular diseases proteins, indicating RA's potential to modulate platelet function. This mechanistic prediction was experimentally validated by showing that RA modulates platelet function through inhibition of protein tyrosine phosphorylation. These observations suggest a potential role of RA on prevention of vascular diseases by inhibiting platelet activation and aggregation, and thereby thrombus-forming potential.

The observed results also suggest multiple avenues through which ability to understand the role of polyphenols could be improved. First, some of the known health benefits of polyphenols might be caused not only by the native molecules, but by their metabolic byproducts. Thus far there is a lack data about colonic degradation, liver metabolism, bioavailability, and interaction with proteins of specific polyphenols or their metabolic byproducts. Once available, future experimental data on protein interactions with polyphenol byproducts and conjugates can be incorporated in the proposed framework, further improving the accuracy of predictions. The lack of some data does not invalidate the findings presented here, since previous studies report the presence of unmetabolized polyphenols in blood; and it has been hypothesized that, in some instances, deconjugation of liver metabolites occurs in specific tissues or cells. Second, considering that several experimental studies of polyphenol bioefficacy have been observed in in vitro and in vivo models, the proposed framework might help interpret literature evidence, possibly even allowing the exclusion of chemical candidates when considering the health benefits provided by a given food in epidemiological association studies.

The low bioavailability of some polyphenols might still present a major challenge when considering the therapeutic utility of these molecules. However, in the same way that the polyphenol phlorizin led to the discovery of new strategies for disease treatment resulting in the development of new compounds with higher efficacy, it is believed that the present methodology can help identify other polyphenol-based candidates for drug development. Future research on the factors limiting polyphenol bioavailability might offer strategies for maximizing the bioavailability of those with potential for health benefits.

The methodology introduced here offers a foundation for the mechanistic interpretation of alternative pathways through which polyphenols can affect health, e.g., the combined effect of different polyphenols and their interaction with drugs. Finally, this methodology can be applied to other food-related chemicals, providing a framework by which to understand their health effects. Future research in this area may help also account for the way that food-related chemicals affect endogenous metabolic reactions, impacting not only signaling pathways, but also catabolic and anabolic processes. Taken together, the proposed network-based framework has the potential to reveal systematically the mechanism of action underlying the health benefits of polyphenols, offering a logical, rational strategy for mechanism-based drug development of food-based compounds.

TABLE 1
Top 20 Predicted Therapeutic Associations
Between EGCG and Human Diseases

| | Distance | Significance |
|---|---|---|
| Disease | d_{c} | Z_{dc} |
| nervous system diseases | 1.13 | −1.72 |
| nutritional and metabolic diseases | 1.25 | −1.45 |
| metabolic diseases | 1.25 | −1.41 |
| cardiovascular diseases | 1.27 | −2.67 |
| immune system diseases | 1.29 | −1.31 |
| vascular diseases | 1.33 | −3.47 |
| digestive system diseases | 1.33 | −1.57 |
| neurodegenerative diseases | 1.37 | −1.71 |
| central nervous system diseases | 1.41 | −0.54 |
| autoimmune diseases | 1.41 | −1.30 |
| gastrointestinal diseases | 1.43 | −1.02 |
| brain diseases | 1.43 | −0.89 |
| intestinal diseases | 1.49 | −1.08 |
| inflammatory bowel diseases | 1.54 | −2.10 |
| bone diseases | 1.54 | −1.18 |
| gastroenteritis | 1.54 | −1.92 |
| demyelinating diseases | 1.54 | −1.78 |
| glucose metabolism disorders | 1.54 | −1.58 |
| heart diseases | 1.56 | −1.20 |
| diabetes mellitus | 1.56 | −1.66 |

Diseases were ordered according to the network distance (d_{c}) of their proteins to EGCG targets and diseases with relative distance Z_{dc} > −0.5 were removed.

TABLE 2
Top Ranked Polyphenols

| | | AUC | | Concentration in | N Mapped | LCC |
|---|---|---|---|---|---|---|
| Polyphenol | AUC | CI* | Precision** | Blood*** | Targets | Size |
| Coumarin | 0.93 | [0.86-0.98] | 0.6 | | 7 | 1 |
| Piceatannol | 0.86 | [0.77-0.94] | 0.6 | | 39 | 23 |
| Genistein | 0.82 | [0.75-0.89] | 0.7 | [0.006-0.525 μM] | 18 | 6 |
| Ellagic acid | 0.79 | [0.63-0.92] | 0.6 | | 42 | 19 |
| (−)-epigallocatechin | 0.78 | [0.70-0.86] | 0.8 | | 51 | 17 |
| 3-o-gallate | | | | | | |
| Isoliquiritigemn | 0.75 | [0.77-0.94] | 0.6 | | 10 | 8 |
| Resveratrol | 0.75 | [0.66-0.82] | 1 | | 63 | 25 |
| Pterostilbene | 0.73 | [0.61-0.84] | 0.6 | | 5 | 2 |
| Quercetin | 0.73 | [0.64-0.81] | 1 | [0.022-0.080 μM] | 216 | 140 |
| (−)-epicatechin | 0.65 | [0.49-0.80] | 0.8 | 0.625 μM | 11 | 3 |

Table showing polyphenols with AUC > 0.6 and Precision > 0.6.
*Confidence intervals calculated with 2000 bootstraps with replacement and sample size of 50% of the diseases (150/299)
**Precision was calculated based on the top 10 polyphenols after their ranking based on the distance (d_{c}) of their targets to the disease proteins and considering only predictions with Z-score < −0.5.
***Concentrations of polyphenols in blood were retrieved from the Human Metabolome Database (HMDB)

TABLE 3
Polyphenols Proximal to Vascular Diseases

| | Number | | |
|---|---|---|---|
| | of Protein | | |
| chemical | Targets | d_{c} | Z_{dc} |
| gallic acid | 1 | 0.00 | −3.02 |
| prunetin | 1 | 0.00 | −2.82 |
| daidzin | 1 | 0.00 | −2.82 |
| punicalagin | 1 | 1.00 | −1.09 |
| kaempferol 3-o-galactoside | 1 | 1.00 | −1.75 |
| juglone | 2 | 1.00 | −1.92 |
| kaempferol 3-o-glucoside | 2 | 1.00 | −2.10 |
| 4-methylcatechol | 2 | 1.00 | −1.01 |
| rosmarinic acid | 3 | 1.00 | −1.38 |
| xanthotoxin | 3 | 1.33 | −2.05 |
| daidzein | 3 | 0.66 | −2.48 |
| umbelliferone | 3 | 1.33 | −1.50 |
| 1,4-naphthoquinone | 4 | 1.25 | −1.51 |
| 3-caffeoylquinic acid | 9 | 1.66 | −1.19 |
| isoliquiritigenin | 10 | 1.70 | −0.76 |
| chrysin | 12 | 1.50 | −0.64 |
| cinnamic acid | 15 | 1.46 | −1.37 |
| caffeic acid | 16 | 1.56 | −0.77 |
| genistein | 18 | 1.44 | −0.97 |
| 3-phenylpropionic acid | 18 | 1.72 | −0.53 |
| butein | 19 | 1.52 | −1.97 |
| myricetin | 34 | 1.47 | −0.60 |
| piceatannol | 39 | 1.05 | −2.64 |
| ellagic acid | 42 | 1.45 | −1.09 |
| (-)-epigallocatechin 3-o-gallate | 51 | 1.33 | −3.47 |
| phenol | 98 | 1.50 | −3.05 |
| quercetin | 216 | 1.37 | −2.18 |

TABLE 4
Summary of Polyphenols Evaluated in this Study. Name, class, subclass and PubChem IDs for polyphenols.
The table also shows the number of polyphenol protein targets mapped in the human interactome,
the size of the largest connected component (LCC) formed by them and
z-score for the LCC size. The columns min (μM) and max (μM) report the minimum and maximum polyphenol
concentrations detected in blood according to Human Metabolome Database (HMDB).

| | | | Pub | N | | | | | |
|---|---|---|---|---|---|---|---|---|---|
| | | | Chem | Targets | | Z- | Min | Max | |
| Name | Class | Subclass | IDs | Mapped | LCC | score | (μM) | (μM) | HMDB |
| quercetin | Flavonoids | Flavonols | 5280343 | 216 | 140 | −1.30 | | | |
| resveratrol | Stilbenes | Stilbenes | 445154 | 63 | 25 | −2.79 | | | |
| piceatannol | Stilbenes | Stilbenes | 4813 | 39 | 23 | −1.91 | | | |
| ellagic acid | Phenolic | Hydroxy- | 5281855 | 42 | 19 | −1.54 | 0.067 | 0.067 | HMDB0002899 |
| | acids | benzoic acids | | | | | | | |
| phenol | Other | Other | 996 | 98 | 19 | 3.79 | 0.86 | 6.38 | HMDB0000228 |
| | polyphenols | polyphenols | | | | | | | |
| (−)- | Flavonoids | Flavanols | 65064 | 51 | 17 | −2.39 | | | |
| epigallocatechin | | | | | | | | | |
| 3-O-gallate | | | | | | | | | |
| butein | Flavonoids | Chalcones | 5281222 | 19 | 8 | −1.10 | | | |
| apigenin | Flavonoids | Flavones | 5280443 | 25 | 8 | −1.64 | 0.0106 | 0.127 | HMDB0002124 |
| luteolin | Flavonoids | Flavones | 5280445 | 32 | 8 | −1.94 | | | |
| iso- | Flavonoids | Isoflavonoids | 638278 | 10 | 8 | −3.68 | | | |
| liquiritigenin | | | | | | | | | |
| kaempferol | Flavonoids | Flavonols | 5280863 | 37 | 8 | −0.39 | | | |
| 3-caffeoyl- | Phenolic | Hydroxy- | 9476 | 9 | 8 | −4.25 | | | |
| quinic | acids | cinnamic acids | | | | | | | |
| acid | | | | | | | | | |
| genistein | Flavonoids | Isoflavonoids | 5280961 | 18 | 6 | −0.14 | 0.00022 | 0.525 | HMDB0003217 |
| myricetin | Flavonoids | Flavonols | 5281672 | 34 | 6 | −0.67 | 45 | 45 | HMDB0002755 |
| chrysin | Flavonoids | Flavones | 5281607 | 12 | 4 | −2.11 | | | |
| quercetin 3-O- | Flavonoids | Flavonols | 5280804 | 7 | 3 | −1.99 | | | |
| glucoside | | | | | | | | | |
| cinnamic acid | Phenolic | Hydroxy- | 444539 | 15 | 3 | −0.24 | | | |
| | acids | cinnamic acids | | | | | | | |
| (−)-epicatechin | Flavonoids | Flavanols | 72276 | 11 | 3 | −2.36 | 0.625 | 0.625 | HMDB00017871 |
| pterostilbene | Stilbenes | Stilbenes | 5281727 | 5 | 2 | −1.25 | | | |
| ferulic acid | Phenolic | Hydroxy- | 709 | 10 | 2 | −0.80 | | | |
| | acids | cinnamic acids | | | | | | | |
| baicalein | Flavonoids | Flavones | 5281605 | 9 | 2 | 0.57 | | | |
| coumestrol | Other | Other | 5281707 | 3 | 2 | −1.11 | 0.0123 | 0.0123 | HMDB0002326 |
| | polyphenols | polyphenols | | | | | | | |
| p-coumaric | Phenolic | Hydroxy- | 322 | 13 | 2 | −0.91 | | | |
| acid | acids | cinnamic acids | | | | | | | |
| daidzein | Flavonoids | Isoflavonoids | 5281708 | 3 | 2 | −1.14 | | | |
| 3-phenyl- | Phenolic | Hydroxyphenyl- | 107 | 18 | 2 | −0.22 | 0.504 | 44.348 | HMDB0000764 |
| propionic | acids | propanoic | | | | | | | |
| acid | | acids | | | | | | | |
| caffeic acid | Phenolic | Hydroxy- | 689043 | 16 | 2 | −0.22 | | | |
| | acids | cinnamic acids | | | | | | | |
| (−)-epicatechin | Flavonoids | Flavanols | 107905 | 7 | 2 | −0.23 | | | |
| 3-O-gallate | | | | | | | | | |
| guaiacol | Other | Methoxy- | 460 | 2 | 1 | 0.37 | 8.5 | 8.5 | HMDB0001398 |
| | polyphenols | phenols | | | | | | | |
| xanthotoxin | Other | Furano- | 4114 | 3 | 1 | −0.35 | | | |
| | polyphenols | coumarins | | | | | | | |
| phenylacetic | Phenolic | Hydroxy- | 999 | 1 | 1 | | 10.28 | 80.36 | HMDB0000209 |
| acid | acids | phenylacetic | | | | | | | |
| | | acids | | | | | | | |
| quercetin 3-O- | Flavonoids | Flavonols | 5280805 | 5 | 1 | 0.55 | | | |
| rutinoside | | | | | | | | | |
| phloretin | Flavonoids | Dihydro- | 4788 | 2 | 1 | 0.04 | | | |
| | | chalcones | | | | | | | |
| kaempferol 3- | Flavonoids | Flavonols | 5282102 | 2 | 1 | 0.91 | | | |
| O-glucoside | | | | | | | | | |
| schisantherin a | Lignans | Lignans | 151529 | 1 | 1 | | | | |
| 4-methyl- | Other | Alkylphenols | 9958 | 2 | 1 | −0.33 | | | |
| catechol | polyphenols | | | | | | | | |
| thymol | Other | Phenolic | 6989 | 1 | 1 | | | | |
| | polyphenols | terpenes | | | | | | | |
| psoralen | Other | Furano- | 6199 | 3 | 1 | −1.37 | | | |
| | polyphenols | coumarins | | | | | | | |
| daidzin | Flavonoids | Isoflavonoids | 107971 | 1 | 1 | | | | |
| naringenin | Flavonoids | Flavanones | 439246 | 2 | 1 | −0.78 | 0.00815 | 0.02 | HMDB0002670 |
| prunetin | Flavonoids | Isoflavonoids | 5281804 | 1 | 1 | | | | |
| biochanin a | Flavonoids | Isoflavonoids | 5280373 | 1 | 1 | | | | |
| quercetin 3-O- | Flavonoids | Flavonols | 5274585 | 1 | 1 | | | | |
| glucuronide | | | | | | | | | |
| luteolin 6-c- | Flavonoids | Flavones | 114776 | 1 | 1 | | | | |
| glucoside | | | | | | | | | |
| 2,3-dihydroxy- | Phenolic | Hydroxy- | 19 | 1 | 1 | | 0.129 | 0.129 | HMDB0000397 |
| benzoic acid | acids | benzoic | | | | | | | |
| | | acids | | | | | | | |
| esculetin | Other | Hydroxy- | 5281416 | 1 | 1 | | | | |
| | polyphenols | coumarins | | | | | | | |
| rosmarinic acid | Phenolic | Hydroxy- | 5281792 | 3 | 1 | −0.48 | | | |
| | acids | cinnamic acids | | | | | | | |
| 2-hydroxy- | Phenolic | Hydroxy- | 338 | 11 | 1 | 1.00 | 0.02 | 0.02 | HMDB0001895 |
| benzoic | acids | benzoic | | | | | | | |
| acid | | acids | | | | | | | |
| schisandrin b | Lignans | Lignans | 108130 | 1 | 1 | | | | |
| kaempferol 3- | Flavonoids | Flavonols | 5488283 | 1 | 1 | | | | |
| O-galactoside | | | | | | | | | |
| theaflavin | Flavonoids | Flavanols | 114777 | 1 | 1 | | | | |
| coumarin | Other | Hydroxy- | 323 | 7 | 1 | 1.34 | | | |
| | polyphenols | coumarins | | | | | | | |
| naringin | Flavonoids | Flavanones | 442428 | 1 | 1 | | | | |
| punicalagin | Phenolic | Hydroxy- | 16129869 | 1 | 1 | | | | |
| | acids | benzoic acids | | | | | | | |
| umbelliferone | Other | Hydroxy- | 5281426 | 3 | 1 | 0.14 | | | |
| | polyphenols | coumarins | | | | | | | |
| gallic acid | Phenolic | Hydroxy- | 370 | 1 | 1 | | | | |
| | acids | benzoic | | | | | | | |
| | | acids | | | | | | | |
| 1,4- | Other | Naphto- | 8530 | 4 | 1 | −1.95 | | | |
| naphtoquinone | polyphenols | quinones | | | | | | | |
| carvacrol | Other | Phenolic | 10364 | 2 | 1 | −0.04 | | | |
| | polyphenols | terpenes | | | | | | | |
| hesperetin | Flavonoids | Flavanones | 72281 | 1 | 1 | | | | |
| juglone | Other | Naphto- | 3806 | 2 | 1 | −1.23 | | | |
| | polyphenols | quinones | | | | | | | |
| phloridzin | Flavonoids | Dihydro- | 4789 | 5 | 1 | 1.00 | | | |
| | | chalcones | | | | | | | |
| isorhamnetin | Flavonoids | Flavonols | 5281654 | 4 | 1 | −1.34 | 0.0388 | 0.157 | HMDB0002655 |
| scutellarein | Flavonoids | Flavones | 5281697 | 2 | 1 | 0.58 | | | |
| galangin | Flavonoids | Flavonols | 5281616 | 6 | 1 | 0.28 | | | |
| nobiletin | Flavonoids | Flavones | 72344 | 1 | 1 | | | | |
| galloyl glucose | Phenolic | Hydroxy- | 124021 | 1 | 1 | | | | |
| | acids | benzoic | | | | | | | |
| | | acids | | | | | | | |

TABLE 5
Predicted Gastrointestinal (GI) Absorption and Bioavailability.
Predictions obtained from the SwissADME webserver.
The column ‘bioavailability score’ reports the probability of a
compound to have at least 10% oral bioavailability in rat or
of having measurable Caco-2 permeability.

| | | | Bio- |
|---|---|---|---|
| | PubChem | GI | availability |
| Polyphenol | ID | absorption | Score |
| carvacrol | 10364 | High | 0.55 |
| 3-phenylpropionic acid | 107 | High | 0.56 |
| (-)-epicatechin 3-O-gallate | 107905 | Low | 0.55 |
| daidzin | 107971 | Low | 0.55 |
| schisandrin b | 108130 | High | 0.55 |
| luteolin 6-c-glucoside | 114776 | Low | 0.17 |
| theaflavin | 114777 | Low | 0.17 |
| galloyl glucose | 124021 | Low | 0.55 |
| schisantherin a | 151529 | High | 0.55 |
| punicalagin | 16129869 | Low | 0.17 |
| 2,3-dihydroxybenzoic acid | 19 | High | 0.56 |
| p-coumaric acid | 322 | High | 0.56 |
| coumarin | 323 | High | 0.55 |
| 2-hydroxybenzoic acid | 338 | High | 0.56 |
| gallic acid | 370 | High | 0.56 |
| juglone | 3806 | High | 0.55 |
| xanthotoxin | 4114 | High | 0.55 |
| naringenin | 439246 | High | 0.55 |
| naringin | 442428 | Low | 0.17 |
| cinnamic acid | 444539 | High | 0.56 |
| resveratrol | 445154 | High | 0.55 |
| guaiacol | 460 | High | 0.55 |
| phloretin | 4788 | High | 0.55 |
| phloridzin | 4789 | Low | 0.55 |
| piceatannol | 4813 | High | 0.55 |
| quercetin 3-O-glucuronide | 5274585 | Low | 0.11 |
| quercetin | 5280343 | High | 0.55 |
| biochanin a | 5280373 | High | 0.55 |
| apigenin | 5280443 | High | 0.55 |
| luteolin | 5280445 | High | 0.55 |
| quercetin 3-O-glucoside | 5280804 | Low | 0.17 |
| quercetin 3-O-rutinoside | 5280805 | Low | 0.17 |
| kaempferol | 5280863 | High | 0.55 |
| genistein | 5280961 | High | 0.55 |
| butein | 5281222 | High | 0.55 |
| esculetin | 5281416 | High | 0.55 |
| umbelliferone | 5281426 | High | 0.55 |
| baicalein | 5281605 | High | 0.55 |
| chrysin | 5281607 | High | 0.55 |
| galangin | 5281616 | High | 0.55 |
| isorhamnetin | 5281654 | High | 0.55 |
| myricetin | 5281672 | Low | 0.55 |
| scutellarein | 5281697 | High | 0.55 |
| coumestrol | 5281707 | High | 0.55 |
| daidzein | 5281708 | High | 0.55 |
| pterostilbene | 5281727 | High | 0.55 |
| rosmarinic acid | 5281792 | Low | 0.56 |
| prunetin | 5281804 | High | 0.55 |
| ellagic acid | 5281855 | High | 0.55 |
| kaempferol 3-O-glucoside | 5282102 | Low | 0.17 |
| kaempferol 3-O-galactoside | 5488283 | Low | 0.17 |
| psoralen | 6199 | High | 0.55 |
| isoliquiritigenin | 638278 | High | 0.55 |
| (-)-epigallocatechin 3-O-gallate | 65064 | Low | 0.17 |
| caffeic acid | 689043 | High | 0.56 |
| thymol | 6989 | High | 0.55 |
| ferulic acid | 709 | High | 0.56 |
| (-)-epicatechin | 72276 | High | 0.55 |
| hesperetin | 72281 | High | 0.55 |
| nobiletin | 72344 | High | 0.55 |
| 1,4-naphtoquinone | 8530 | High | 0.55 |
| 3-caffeoylquinic acid | 9476 | Low | 0.11 |
| 4-methylcatechol | 9958 | High | 0.55 |
| phenol | 996 | High | 0.55 |
| phenylacetic acid | 999 | High | 0.56 |

TABLE 6
Polyphenols Proximal to Vascular Diseases.

| | |
|---|---|
| apigenin | naringenin |
| caffeic acid | naringin |
| coumarin | nobiletin |
| coumestrol | piceatannol |
| daidzein | prunetin |
| epicatechin | pterostilbene |
| (-)-epicatechin 3-O-gallate | quercetin |
| (-)-epigallocatechin 3-O-gallate | resveratrol |
| genistein | rosmarinic acid |
| isoliquiritigenin | umbelliferone |
| myricetin | xanthotoxin |

### REFERENCES

1. Khera, A. V. et al. Genetic risk, adherence to a healthy lifestyle, and coronary disease. N. Engl. J. Med. 375, 2349-2358 (2016).

2. Arts, I. C. W. & Hollman, P. C. H. Polyphenols and disease risk in epidemiologic studies. Am. J. Clin. Nutr. 81, 317S-325S (2005).

3. Wang, X., Ouyang, Y. Y., Liu, J. & Zhao, G. Flavonoid intake and risk of CVD: a systematic review and meta-analysis of prospective cohort studies. Br. J. Nutr. 111, 1-11 (2014).

4. Neveu, V. et al. Phenol-Explorer: an online comprehensive database on polyphenol contents in foods. Database 2010, bap024 (2010).

5. Pérez-Jiménez, J., Neveu, V., Vos, F. & Scalbert, A. Systematic analysis of the content of 502 polyphenols in 452 foods and beverages: an application of the phenol-explorer database. J. Agric. Food Chem. 58, 4959-4969 (2010).

6. Zhang, H. & Tsao, R. Dietary polyphenols, oxidative stress and antioxidant and anti-inflammatory effects. Curr. Opin. Food Sci. 8, 33-42 (2016).

7. Boly, R. et al. Quercetin inhibits a large panel of kinases implicated in cancer cell biology. Int. J. Oncol. 38, 833-842 (2011).

8. Lacroix, S. et al. A computationally driven analysis of the polyphenol-protein interactome. Sci. Rep. 8, 2232 (2018).

9. Hanhineva, K. et al. Impact of dietary polyphenols on carbohydrate metabolism. Int. J. Mol. Sci. 11, 1365-1402 (2010).

10. Hervert-Hernández, D. & Goni, I. Dietary polyphenols and human gut microbiota: a review. Food Rev. Int. 27, 154-169 (2011).

11. Zhang, S. et al. Dietary pomegranate extract and inulin affect gut microbiome differentially in mice fed an obesogenic diet. Anaerobe 48, 184-193 (2017).

12. Thazhath, S. S. et al. Administration of resveratrol for 5 wk has no effect on glucagon-like peptide 1 secretion, gastric emptying, or glycemic control in type 2 diabetes: a randomized controlled trial. Am. J. Clin. Nutr. 103, 66-70 (2016).

13. Bhatt, J. K., Thomas, S. & Nanjan, M. J. Resveratrol supplementation improves glycemic control in type 2 diabetes mellitus. Nutr. Res. 32, 537-541 (2012).

14. Sharma, A. et al. A disease module in the interactome explains disease heterogeneity, drug response and captures novel pathways and genes in asthma. Hum. Mol. Genet. 24, 3005-3020 (2014).

15. Menche, J. et al. Disease networks. Uncovering disease-disease relationships through the incomplete interactome. Science 347, 1257601 (2015).

16. Guney, E., Menche, J., Vidal, M. & Barabási, A.-L. Network-based in silico drug efficacy screening. Nat. Commun. 7, 10331 (2016).

17. Cheng, F. et al. Network-based approach to prediction and population-based validation of in silico drug repurposing. Nat. Commun. 9, 2691 (2018).

18. Kovács, I. A. et al. Network-based prediction of protein interactions. Nat. Commun. 10, 1240 (2019).

19. Sarkar, F. H., Li, Y., Wang, Z. & Kong, D. Cellular signaling perturbation by natural products. Cell. Signal. 21, 1541-1547 (2009).

20. Iso, H. et al. The relationship between green tea and total caffeine intake and risk for self-reported type 2 diabetes among Japanese adults. Ann. Intern. Med. 144, 554-562 (2006).

21. Song, Y., Manson, J. E., Buring, J. E., Sesso, H. D. & Liu, S. Associations of dietary flavonoids with risk of type 2 diabetes, and markers of insulin resistance and systemic inflammation in women: a prospective study and cross-sectional analysis. J. Am. Coll. Nutr. 24, 376-384 (2005).

22. Keske, M. A. et al. Vascular and metabolic actions of the green tea polyphenol epigallocatechin gallate. Curr. Med. Chem. 22, 59-69 (2015).

23. Wolfram, S. et al. Epigallocatechin gallate supplementation alleviates diabetes in rodents. J. Nutr. 136, 2512-2518 (2006).

24. Davis, A. P. et al. The comparative toxicogenomics database: update 2019. Nucleic Acids Res. 47, D948-D954 (2019).

25. Muthu, R., Selvaraj, N. & Vaiyapuri, M. Anti-inflammatory and proapoptotic effects of umbelliferone in colon carcinogenesis. Hum. Exp. Toxicol. 35, 1041-1054 (2016).

26. Muthu, R. & Vaiyapuri, M. Synergistic and individual effects of umbelliferone with 5-fluorouracil on tumor markers and antioxidant status of rat treated with 1,2-dimethylhydrazine. Biomed. Aging Pathol. 3, 219-227 (2013).

27. Subramanian, A. et al. A next generation connectivity map: L1000 platform and the first 1,000,000 profiles. Cell 171, 1437-1452.e17 (2017).

28. Grover, S. P., Bergmeier, W. & Mackman, N. Platelet signaling pathways and new inhibitors. Arterioscler. Thromb. Vasc. Biol. 38, e28-e35 (2018).

29. Moco, S., Martin, F. P. J. & Rezzi, S. Metabolomics view on gut microbiome modulation by polyphenol-rich foods. J. Proteome Res. 11, 4781-4790 (2012).

30. van Duynhoven, J. et al. Metabolic fate of polyphenols in the human superorganism. Proc. Natl Acad. Sci. USA 108, 4531-4538 (2011).

31. Ottaviani, J. I., Heiss, C., Spencer, J. P. E., Kelm, M. & Schroeter, H. Recommending flavanols and procyanidins for cardiovascular health: revisited. Mol. Aspects Med. 61, 63-75 (2018).

32. Stalmach, A., Troufflard, S., Serafini, M. & Crozier, A. Absorption, metabolism and excretion of Choladi green tea flavan-3-ols by humans. Mol. Nutr. Food Res. 53, S44-53 (2009).

33. Meng, X. et al. Identification and characterization of methylated and ring-fission metabolites of tea catechins formed in humans, mice, and rats. Chem. Res. Toxicol. 15, 1042-1050 (2002).

34. Perez-Vizcaino, F., Duarte, J. & Santos-Buelga, C. The flavonoid paradox: conjugation and deconjugation as key steps for the biological activity of flavonoids. J. Sci. Food Agric. 92, 1822-1825 (2012).

35. Shimoi, K. & Nakayama, T. Glucuronidase deconjugation in inflammation. Methods Enzymol. 400, 263-272 (2005).

36. Kaneko, A. et al. Glucuronides of phytoestrogen flavonoid enhance macrophage function via conversion to aglycones by β-glucuronidase in macrophages. Immun. Inflamm. Dis. 5, 265-279 (2017).

37. Cheng, F., Kovács, I. A. & Barabási, A.-L. Network-based prediction of drug combinations. Nat. Commun. 10, 1197 (2019).

38. Smalley, J. L., Gant, T. W. & Zhang, S.-D. Application of connectivity mapping in predictive toxicology based on gene-expression similarity. Toxicology 268, 143-146 (2010).

39. Lamb, J. et al. The Connectivity Map: using gene-expression signatures to connect small molecules, genes, and disease. Science 313, 1929-1935 (2006).

40. Amanzadeh, E. et al. Quercetin conjugated with superparamagnetic iron oxide nanoparticles improves learning and memory better than free quercetin via interacting with proteins involved in LTP. Sci. Rep. 9, 6876 (2019).

41. Shaikh, J., Ankola, D. D., Beniwal, V., Singh, D. & Kumar, M. N. V. R. Nanoparticle encapsulation improves oral bioavailability of curcumin by at least 9-fold when compared to curcumin administered with piperine as absorption enhancer. Eur. J. Pharm. Sci. 37, 223-230 (2009).

42. Chao, E. C. & Henry, R. R. SGLT2 inhibition-A novel strategy for diabetes treatment. Nat. Rev. Drug Discov. 9, 551-559 (2010).

43. Caldera, M. et al. Mapping the perturbome network of cellular perturbations. Nat. Commun. 10, 5140 (2019).

44. Jensen, K., Ni, Y., Panagiotou, G. & Kouskoumvekaki, I. Developing a molecular roadmap of drug-food interactions. PLoS Comput. Biol. 11, e1004048 (2015).

45. Rolland, T. et al. A proteome-scale map of the human interactome network. Cell 159, 1212-1226 (2014).

46. Cheng, F., Jia, P., Wang, Q. & Zhao, Z. Quantitative network mapping of the human kinome interactome reveals new clues for rational kinase inhibitor discovery and individualized cancer therapy. Oncotarget 5, 3697-3710 (2014).

47. Calçada, D. et al. The role of low-grade inflammation and metabolic flexibility in aging and nutritional modulation thereof: a systems biology approach. Mech. Ageing Dev. http://dx.doi.org/10.1016/j.mad.2014.01.004 (2014).

48. Hornbeck, P. V. et al. PhosphoSitePlus, 2014: mutations, PTMs and recalibrations. Nucleic Acids Res. 43, D512-D520 (2015).

49. Li, T. et al. A scored human protein-protein interaction network to catalyze genomic interpretation. Nat. Methods 14, 61-64 (2016).

50. Chatr-Aryamontri, A. et al. The BioGRID interaction database: 2017 update. Nucleic Acids Res. 45, D369-D379 (2017).

51. Cowley, M. J. et al. PINA v2.0: mining interactome modules. Nucleic Acids Res. 40, D862-D865 (2012).

52. Peri, S. et al. Human protein reference database as a discovery resource for proteomics. Nucleic Acids Res. 32, D497-D501 (2004).

53. Orchard, S. et al. The MIntAct project-IntAct as a common curation platform for 11 molecular interaction databases. Nucleic Acids Res. 42, D358-D363 (2014).

54. Breuer, K. et al. InnateDB: systems biology of innate immunity and beyond-recent updates and continuing curation. Nucleic Acids Res. 41, D1228-D1233 (2013).

55. Meyer, M. J., Das, J., Wang, X. & Yu, H. INstruct: a database of high-quality 3D structurally resolved protein interactome networks. Bioinformatics 29, 1577-1579 (2013).

56. Mosca, R., Céol, A. & Aloy, P. Interactome3D: adding structural details to protein networks. Nat. Methods 10, 47-53 (2013).

57. Meyer, M. J. et al. Interactome INSIDER: a structural interactome browser for genomic studies. Nat. Methods 15, 107-114 (2018).

58.Fazekas, D. et al. SignaLink 2—a signaling pathway resource with multi-layered regulatory networks. BMC Syst. Biol. https://doi.org/10.1186/1752-0509-7-7 (2013).

59. Huttlin, E. L. et al. Architecture of the human interactome defines protein communities and disease networks. Nature 545, 505-509 (2017).

60. Szklarczyk, D. et al. STRING v10: protein-protein interaction networks, integrated over the tree of life. Nucleic Acids Res. 43, D447-D452 (2015).

61. Subramanian, A. et al. Gene set enrichment analysis: a knowledge-based approach for interpreting genome-wide expression profiles. Proc. Natl Acad. Sci. USA 102, 15545-15550 (2005).

62. Roweth, H. G. et al. Two novel, putative mechanisms of action for citalopram-induced platelet inhibition. Sci. Rep. 8, 16677 (2018).

63. Roweth, H. G. et al. Citalopram inhibits platelet function independently of SERT-mediated 5-HT transport. Sci. Rep. 8, 3494 (2018).

64. Nath, S., Bachani, M., Harshavardhana, D. & Steiner, J. P. Catechins protect neurons against mitochondrial toxins and HIV proteins via activation of the BDNF pathway. J. Neurovirol. 18, 445-455 (2012).

65. Park, K.-S. et al. (−)-Epigallocatethin-3-O-gallate counteracts caffeine-induced hyperactivity: evidence of dopaminergic blockade. Behav. Pharmacol. 21, 572-575 (2010).

66. Ramesh, E., Geraldine, P. & Thomas, P. A. Regulatory effect of epigallocatechin gallate on the expression of C-reactive protein and other inflammatory markers in an experimental model of atherosclerosis. Chem.Biol. Interact. 183, 125-132 (2010).

67. Han, S. G., Han, S.-S., Toborek, M. & Hennig, B. EGCG protects endothelial cells against PCB 126-induced inflammation through inhibition of AhR and induction of Nrf2-regulated genes. Toxicol. Appl. Pharmacol. 261, 181-188 (2012).

68. Sheng, R., Gu, Z.-L. & Xie, M.-L. Epigallocatechin gallate, the major component of polyphenols in green tea, inhibits telomere attrition mediated cardiomyocyte apoptosis in cardiac hypertrophy. Int. J. Cardiol. 162, 199-209 (2013).

69. Devika, P. T. & Stanely Mainzen Prince, P. (−)-Epigallocatechin gallate protects the mitochondria against the deleterious effects of lipids, calcium and adenosine triphosphate in isoproterenol induced myocardial infarcted male Wistar rats. J. Appl. Toxicol. 28, 938-944 (2008).

70. Yi, Q.-Y. et al. Chronic infusion of epigallocatechin-3-O-gallate into the hypothalamic paraventricular nucleus attenuates hypertension and sympathoexcitation by restoring neurotransmitters and cytokines. Toxicol. Lett. 262, 105-113 (2016).

71. Devika, P. T. & Prince, P. S. M. Preventive effect of (−)-epigallocatechin-gallate (EGCG) on lysosomal enzymes in heart and subcellular fractions in isoproterenol-induced myocardial infarcted Wistar rats. Chem. Biol. Interact. 172, 245-252 (2008).

72. Hushmendy, S. et al. Select phytochemicals suppress human T-lymphocytes and mouse splenocytes suggesting their use in autoimmunity and transplantation. Nutr. Res. 29, 568-578 (2009).

73. Shen, K. et al. Epigallocatechin 3-gallate ameliorates bile duct ligation induced liver injury in mice by modulation of mitochondrial oxidative stress and inflammation. PLoS ONE 10, e0126278 (2015).

74. ZHEN, M. et al. Green tea polyphenol epigallocatechin-3-gallate inhibits oxidative damage and preventive effects on carbon tetrachloride-induced hepatic fibrosis. J. Nutr. Biochem. 18, 795-805 (2007).

75. Yasuda, Y. et al. (−)-Epigallocatechin gallate prevents carbon tetrachloride induced rat hepatic fibrosis by inhibiting the expression of the PDGFRβ and IGF-1R. Chem. Biol. Interact. 182, 159-164 (2009).

76. Cao, W. et al. iTRAQ-based proteomic analysis of combination therapy with taurine, epigallocatechin gallate, and genistein on carbon tetrachloride induced liver fibrosis in rats. Toxicol. Lett. 232, 233-245 (2015).

77. Kitamura, M. et al. Epigallocatechin gallate suppresses peritoneal fibrosis in mice. Chem. Biol. Interact. 195, 95-104 (2012).

78. Sakla, M. S. & Lorson, C. L. Induction of full-length survival motor neuron by polyphenol botanical compounds. Hum. Genet. 122, 635-643 (2008).

79. Shimizu, M. et al. (−)-Epigallocatechin gallate inhibits growth and activation of the VEGF/VEGFR axis in human colorectal cancer cells. Chem. Biol. Interact. 185, 247-252 (2010).

The entire teachings of “Predicting the Health Impact of Dietary Polyphenols Using a Network Medicine Framework.” bioRxiv (2020) and “Network medicine framework shows that proximity of polyphenol targets and disease proteins predicts therapeutic effects of polyphenols.” Nature Food 2.3 (2021): 143-155 are incorporated herein by reference.

The entire teachings of PCT Application No. PCT/US2020/034299, filed on May 22, 2020, “Chemical-Disease Perturbation Ranking,” Italo Faria do Valle, Northeastern University, with the replacement drawings filed on Sep. 18, 2020, are incorporated herein by reference.

The teachings of all references cited herein are hereby incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of treating a vascular disease or condition in a subject in need thereof, comprising administering to the subject an effective amount of a vascular disease associated polyphenol, or a pharmaceutically acceptable salt thereof.

2. A method of promoting or supporting vascular health in a subject, comprising administering to the subject an effective amount of a vascular disease associated polyphenol, or a pharmaceutically acceptable salt thereof.

3. The method of claim 2, wherein the subject has been diagnosed as having a vascular disease or condition.

4. The method of claim 3, wherein the vascular disease or condition is ischemic injury, diabetes-induced vascular damage, diabetes mellitus, congestive heart failure, coronary heart disease, cerebral ischemia, restenosis after angioplasty, intermittent claudication, myocardial infarction, dyslipidemia, post-prandial lipemia, peripheral vascular disease, renovascular disease, pulmonary hypertension, vasculitis, acute coronary syndromes, modification of cardiovascular risk, or modified platelet aggregation.

5. The method of claim 3, wherein the vascular disease or condition is coronary heart disease, type 2 diabetes mellitus, cerebral ischemia, or myocardial infarction.

6. A method of inhibiting platelet function comprising contacting platelets with a vascular disease associated polyphenol, or a pharmaceutically acceptable salt thereof.

7. The method of claim 6, wherein the platelets are in vitro.

8. The method of claim 6, wherein the platelets are in vivo.

9. The method of claim 8, wherein the platelets are in a subject, and the method comprises administering to the subject an effective amount of a vascular disease associated polyphenol, or a pharmaceutically acceptable salt thereof.

10. The method of claim 6, wherein the platelet function is platelet aggregation.

11. The method of claim 6, wherein the platelet function is granule secretion.

12. The method of claim 11, wherein granule secretion is alpha-granule secretion or dense granule secretion.

13. The method of claim 2, wherein the vascular disease associated polyphenol is quercetin, (−)-epicatechin-3-o-gallate, (−)-epigallocatechin-3-o-gallate, myricetin, butein, phenol, 3-phenylpropionic acid, quercetin 3-o-glucoside, apigenin, chrysin, piceatannol, isoliquiritigenin, caffeic acid, 3-caffeoylquinic acid, genistein, cinnamic acid, (−)-epicatechin, kaempeferol, resveratrol, luteolin, or ellagic acid, or a pharmaceutically acceptable salt thereof.

14. The method of claim 2, wherein the vascular disease associated polyphenol is pruetin, daidzin, punicalagin, kaempferol 3-o-galactoside, juglone, kaempferol 3-o-glucoside, 4-methylcatechol, rosmarinic acid, xanthotoxin, daidzein, umbelliferone, 1,4-naphthoquinone, 3-caffeoylquinic acid, isoliquiritigenin, chrysin, cinnamic acid, caffeic acid, genistein, 3-phenylpropionic acid, butein, myricetin, piceatannol, piceatannol, ellagic acid, (−)-epigallocatechin 3-o-gallate, phenol, or quercetin, or a pharmaceutically acceptable salt thereof.

15. The method of claim 2, wherein the vascular disease associated polyphenol is gallic acid, 1,4-naphthoquinone, or rosmarinic acid, or a pharmaceutically acceptable salt thereof.

16. The method of claim 2, wherein the vascular disease associated polyphenol is rosmarinic acid, or a pharmaceutically acceptable salt thereof.

17. The method of claim 2, wherein the vascular disease associated polyphenol, or a pharmaceutically acceptable salt thereof, is orally administered.

18. The method of claim 2, wherein the vascular disease associated polyphenol, or a pharmaceutically acceptable salt thereof, is provided in the form of a composition in the form of a dietary supplement, pharmaceutical composition, or medical food.
